(19) United States
(12) Patent Application Publication
(10) Pub. No.: US 2024/0327790 A1
(43) Pub. Date: Oct. 3, 2024

(54) METHODS AND COMPOSITIONS RELATING TO 3-D CULTURES

(71) Applicant: PRESIDENT AND FELLOWS OF HARVARD COLLEGE, Cambridge, MA (US)

(72) Inventors: Maxwell NAGARAJAN, Cambridge, MA (US); Jennifer A. LEWIS, Cambridge, MA (US); Daniel REYNOLDS, Cambridge, MA (US)

(73) Assignee: PRESIDENT AND FELLOWS OF HARVARD COLLEGE, Cambridge, MA (US)

(21) Appl. No.: 18/698,190

(22) PCT Filed: Oct. 4, 2022
(86) PCT No.: PCT/US2022/045626

§ 371 Date: Apr. 3, 2024

Related U.S. Application Data
(60) Provisional application No. 63/253,163, filed on Oct. 7, 2021.
(60) Provisional application No. 63/298,702, filed on Jan. 12, 2022.

(51) Int. Cl. C12N 5/071; C12N 5/077

(52) U.S. Cl. CPC C12N 5/0629; C12N 5/0656; C12N 2513/00; C12N 2533/30; C12N 2533/54; C12N 2533/56; C12N 2533/76

(57) ABSTRACT

Described herein are two-layer 3-D cultures, including those with microfeatures at the boundary between the two layers, as well as methods of making and using such cultures.

### CROSS-REFERENCE TO RELATED APPLICATIONS

This application claims benefit under 35 U.S.C. § 119(e) of U.S. Provisional Application Nos. 63/253,163 filed Oct. 7, 2021 and 63/298,702 filed Jan. 12, 2022, the contents of which are incorporated herein by reference in their entirety.

### TECHNICAL FIELD

The technology described herein relates to methods and compositions for providing layered 3-D cultures, wherein the boundary between layers comprising microfeatures such as rete pegs.

### BACKGROUND

Skin comprises an epidermis layer and a dermis layer. The interface between these two layers is not a smooth plane, but a complex topography with structures known as “rete pegs.” These rete pegs are peg or divot structures (depending on perspective) about 100-400 μm tall. These rete pegs are important to the mechanical properties of skin, the formation of the epidermis, and their shape and size are known to change dramatically when the skin is aged or diseased (e.g., in psoriasis).

The rete pegs are therefore a critical feature of any in vitro model of skin, but most 3D skin models do not exhibit rete pegs. See, e.g., Baltazar et al. Tissue Eng A, 2020; Cubo et al. Biofabrication 2017; Kim et al. Biomaterials 2018; and Pourchet et al. Adv Health Mat 2017. Models that do provide structures mimicking rete pegs suffer from serious disadvantages, such as a lack of a dermis layer, intrinsic damage to the dermis layer, or the lack of a stratified epidermis layer that recapitulates in vivo epidermis. There is therefore a need for a skin model that provides biologically relevant epidermis and dermis layers having an interface comprising rete pegs.

### SUMMARY

Provided herein are methods for preparing multi-layered 3-D cultures where the boundary between the layers comprises microfeatures such as rete pegs. These methods can provide fully functional cell layers on each side of the boundary and do not involve methods that cause damage to the cell or the cell culture media (e.g, laser ablation). In particular, the methods described herein provide 2-layer 3-D cultures comprising dermis and epidermis layers separated by a boundary with microfeatures that recapitulate rete pegs and which demonstrate key characteristics of natural dermis/epidermis, such as a basement membrane.

In one aspect of any of the embodiments, described herein is a method comprising: a) applying at least one face of a stamp onto at least one surface of a pre-gel comprising at least a first type of cell, wherein the at least one face of the stamp comprises one or more microfeatures; b) maintaining contact of the at least one face of the stamp with the at least one surface of the pre-gel while the pre-gel forms a gel; c) separating the at least one face of the stamp and the at least one surface of the gel formed in step b) and thereby providing a gel comprising at least one stamped surface; and d) contacting the at least one stamped surface of the gel resulting from step c) with at least a second type of cell to provide a two layer 3-D culture. In one aspect of any of the embodiments, described herein is a two layer 3-D culture comprising: a first layer comprising at least a first type of cell in a gel; a second layer comprising at least a second type of cell; and a boundary between the first and second layers comprising one or more microfeatures.

In some embodiments of any of the aspects,
a) The at least a first type of cell comprises or is fibroblasts and the at least a second type of cell comprises or is keratinocytes;
b) The at least a first type of cell comprises or is fibroblasts and the at least a second type of cell comprises or is keratinocytes and melanocytes;
c) The at least a first type of cell comprises or is fibroblasts and the at least a second type of cell comprises or is melanocytes;
d) The at least a first type of cell comprises or is fibroblasts and endothelial cells and the at least a second type of cell comprises or is keratinocytes;
e) The at least a first type of cell comprises or is fibroblasts and endothelial cells and the at least a second type of cell comprises or is keratinocytes and melanocytes;
f) The at least a first type of cell comprises or is fibroblasts and endothelial cells and the at least a second type of cell comprises or is melanocytes;
g) The at least a first type of cell comprises or is fibroblasts and immune cells and the at least a second type of cell comprises or is keratinocytes;
h) The at least a first type of cell comprises or is fibroblasts and immune cells and the at least a second type of cell comprises or is keratinocytes and melanocytes;
i) The at least a first type of cell comprises or is fibroblasts and the at least a second type of cell comprises or is induced pluripotent stem cells;
j) The at least a first type of cell comprises or is induced pluripotent stem cells and the at least a second type of cell comprises or is induced pluripotent stem cells;
k) The at least a first type of cell comprises or is fibroblasts and the at least a second type of cell comprises or is cells differentiated from induced pluripotent stem cells;
l) The at least a first type of cell comprises or is cells differentiated from induced pluripotent stem cells and the at least a second type of cell comprises or is cells differentiated from induced pluripotent stem cells;
m) The at least a first type of cell comprises or is fibroblasts and the at least a second type of cell comprises or is heptatocytes;
n) The at least a first type of cell comprises or is fibroblasts and the at least a second type of cell comprises or is intestinal stem cells;
o) The at least a first type of cell comprises or is fibroblasts and the at least a second type of cell comprises or is intestinal epithelial cells;
p) The at least a first type of cell comprises or is fibroblasts and the at least a second type of cell comprises or is tumor cells
q) The at least a first type of cell comprises or is fibroblasts from a psoriasis or is patient and the at least a second type of cell comprises or is keratinocytes from a psoriasis patient; or
r) The at least a first type of cell comprises or is fibroblasts from a diabetic patient and the at least a second type of cell comprises or is keratinocytes from a diabetic patient.

In some embodiments of any of the aspects, the at least a first type of cell is fibroblasts and the at least a second type of cell is keratinocytes. In some embodiments of any of the aspects, the method further comprises a step e) of culturing the 3-D culture until the keratinocytes form a stratified epidermis layer; or the second layer comprises keratinocytes forming a stratified epidermis layer. In some embodiments of any of the aspects, the method further comprises a step e) of culturing the 3-D culture until a basement membrane has formed; or the second layer comprises keratinocytes forming a basement membrane. In some embodiments of any of the aspects, step e) comprises culturing the 3-D culture for at least 2 weeks. In some embodiments of any of the aspects, step e) comprises a first phase of culturing the entire 3-D culture submerged in media and second phase of culturing the 3-D culture such that at least one surface of the keratinocytes are at an air-liquid interface. In some embodiments of any of the aspects, step e) comprises culturing the 3-D culture until the keratinocytes form a layer that is at least 100 μm in depth. In some embodiments of any of the aspects, step e) comprises culturing the 3-D culture until the keratinocytes form a layer that is at least 150 μm in depth.

In some embodiments of any of the aspects, the method further comprises a step e) of culturing the 3-D culture until the second type of cell forms a layer that is at least 50 μm in depth. In some embodiments of any of the aspects, step e) comprises culturing the 3-D culture until the layer is at least 50 μm in depth. In some embodiments of any of the aspects, the second layer is at least 100 μm in depth. In some embodiments of any of the aspects, the second layer is at least 100 μm in depth. In some embodiments of any of the aspects, the second layer is at least 150 μm in depth.

In some embodiments of any of the aspects, the pre-gel, gel, and/or first layer comprises one or more of: collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing. In some embodiments of any of the aspects, the pre-gel, gel, and/or first layer comprises one of the formulations of Table 2 or Table 3. In some embodiments of any of the aspects, the pre-gel, gel, and/or first layer comprises more than 6 mg/mL of collagen, atellocollagen, or telocollagen. In some embodiments of any of the aspects, the pre-gel, gel, and/or first layer comprises more than 8 mg/mL of collagen, atellocollagen, or telocollagen. In some embodiments of any of the aspects, the pre-gel, gel, and/or first layer has a depth of from about 100 μm to 10 mm. In some embodiments of any of the aspects, the pre-gel, gel, and/or first layer has a depth of from about 500 μm to 4 mm. In some embodiments of any of the aspects, the pre-gel, gel, and/or first layer has a depth of from about 500 μm to 2 mm. In some embodiments of any of the aspects, the pre-gel, gel, first layer, and/or culture is, is made or prepared in, or is provided in a Transwell insert.

In some embodiments of any of the aspects, each microfeature extends 1 μm to 1 mm from the face of the stamp and/or into a layer. In some embodiments of any of the aspects, each microfeature extends 100 μm to 400 μm from the face of the stamp and/or into a layer. In some embodiments of any of the aspects, each microfeature has a width and/or length of at least 100 μm. In some embodiments of any of the aspects, each microfeature has a height which is no more than 5× the narrowest dimension of the microfeature. In some embodiments of any of the aspects, each microfeature forms a shape (e.g., on the face of the stamp or in a layer) that is a polyhedron, a hexahedron, a cube, a ridge, a wall, a shape created by introducing a curve or angle into any of the preceding shapes, or a shape created by rounding one or more of the edges of one of the preceding shapes.

In some embodiments of any of the aspects, the method does not comprise exposing the gel or pre-gel to a laser and/or does not comprise laser ablating the gel or pre-gel. In some embodiments of any of the aspects, the pre-gel does not comprise and the culture does not comprise and/or is not contacted with HaCaT keratinocytes. In some embodiments of any of the aspects, the gel, pre-gel, or culture does not comprise PEG-diacrylate and/or is not exposed to ultraviolet light or near-UV light treatment. In some embodiments of any of the aspects, the method does not comprise a step of grafting into or onto an organism. In some embodiments of any of the aspects, the method does not comprise cross-linking components of the gel or pre-gel and/or the culture does not comprise cross-linked components. In some embodiments of any of the aspects, the method does comprise cross-linking components of the gel or pre-gel and/or the culture does comprise cross-linked components. In some embodiments of any of the aspects, the cross-linking is the cross-linking of collagen. In some embodiments of any of the aspects, the cross-linking comprises dehydration and/or thermal treatment. In some embodiments of any of the aspects, the cross-linking comprises the use of a carbodiimde, e.g., 1-ethyl-3-(3-dimethylaminopropyl)carbodiimide hydrochloride (EDC).

In one aspect of any of the embodiments, described herein is a method comprising: preparing a two-layer 3-D culture according to the method described herein; applying a stimulus comprising a candidate agent, mechanical stress, or trauma to the two-layer 3-D culture; and optionally, measuring or observing one or more responses of the two-layer 3-D culture to the stimulus. In some embodiments of any of the aspects, one or more of the cell types comprises or expresses a detectable label and the measuring or observing comprises detecting the label.

In one aspect of any of the embodiments, described herein is a kit comprising one or more stamps comprising at least one face comprising one or more microfeatures. In some embodiments of any of the aspects, the kit further comprises at least one cell culture container comprising at least one cell growth area and the stamp is sized to be inserted into the cell growth area. In some embodiments of any of the aspects, the kit further comprises one or more of: a pre-gel, media, a lift spacer, at least a first type of cell, and at least a second type of cell.

### BRIEF DESCRIPTION OF THE DRAWINGS

FIG. 1 depicts the process of fabrication of skin model with rete pegs, and culture process. A mold is 3D-printed using a commercial 3D-printer. A stamp is molded out of the 3D-print. This stamp is applied on a dermis pre-gel containing fibroblasts and/or other cells present in the dermis of the skin model. The dermis solution forms a gel, and the stamp can be removed. After this step, keratinocytes are added on top of the dermis layer. This is cultured for four days in submerged culture, and then two or more weeks at the air liquid interface, meaning that media is only supplied from below the sample.

FIGS. 2A-2D depict microscopy images of skin models. (FIG. 2A) Cross-section of skin model showing the layers of the epidermis, stained with DAPI for cell nuclei (blue) and wheat germ agglutinin for cell membranes (green). (FIG. 2B) Cross-section of skin model showing appropriate stratification of epidermis, stained with DAPI for cell nuclei (blue), actin (red), cytokeratin 14 for the basal layers (green), and cytokeratin 10 for the suprabasal layers (white). (FIG. 2C) Top-down view of stamped skin model. Cell nuclei (blue) are visible in a square pattern of the stamped features. (FIG. 2D) Cross-section view of one well, stained with DAPI for cell nuclei (blue) and wheat germ agglutinin for cell membranes (green).

FIGS. 3A-3B depict microscopy images of skin models. (FIG. 3A) Top-down view of skin model with ridges. (FIG. 3B) Cross-section view of skin model with ridges. Samples were stained with DAPI for cell nuclei (blue) and wheat germ agglutinin for cell membranes (green).

FIG. 4 depicts the stamp fabrication process.

FIG. 5 depicts images of stamping results.

FIG. 6 depicts images of stamping of gelbrin gel.

FIG. 7 depicts images of a gelbrin gel comprising fibroblasts, 6 days after stamping with a 200 μm microfeature stamp.

FIG. 8 depicts contraction of gels with low collagen plus Geltrex or just high collagen.

FIG. 9 depicts the retention of microfeatures in high collagen gel after 6 days.

FIG. 10 depicts gel stamping with a ridge design.

FIG. 11 depicts microfeature retention in high collagen gels after 19 days of culture.

FIG. 12 depicts microfeature retention in high collagen+fibrin gels after 19 days of culture.

FIGS. 13 and 14 depict illustrative embodiments of stamps.

FIG. 15 depicts a diagram of how samples were cut.

FIG. 16 depicts sample staining. Stained in green is wheat germ agglutinin for the cell membranes, and in blue is DAPI for the cell nuclei. The label in the upper left corner shows which sample each image comes from, with the first number indicating the sample, and the second number indicating the image. For example, 2-1 means this is first image from the second sample. 3 replicates were done at each condition.

FIG. 17 depicts sample staining from the 8 mg/mL telocollagen+5 mg/mL fibrin group from FIG. 16.

FIGS. 18A-18C depict the generation of human skin models with rete peg-like structures. (FIG. 18A) depicts a schematic of method. (FIG. 18B) depicts imagees of growth of EDC-crosslinked skin model overtime. (FIG. 18C) depicts images of growth of skin model with no crosslinking over time.

FIGS. 19A-19F demonstrate that telocollagen-fibrin gels stamped with 400-500 μm features show degraded but lasting features over two weeks of culture, while stamps with smaller features decay. Gels crosslinked with EDC maintain features over the two-week culture period. All gels have a composition of 7.9 mg/mL telocollagen and 4.6 mg/mL fibrin. (FIG. 19A) Representative images of samples stamped with 200 μm features at day 0, at day 14 with no crosslinking, and at day 14 with EDC crosslinking. (FIG. 19B) Representative images of samples stamped with 300 μm features at day 0, at day 14 with no crosslinking, and at day 14 with EDC crosslinking. (FIG. 19C) Representative images of samples stamped with 400 μm features at day 0, at day 14 with no crosslinking, and at day 14 with EDC crosslinking. (FIG. 19D) Representative images of samples stamped with 500 μm features at day 0, at day 14 with no crosslinking, and at day 14 with EDC crosslinking. (FIG. 19E, FIG. 19F) Interdigitation index, defined as the length of the interface between the epidermis and dermis divided by the straight-line distance, is plotted for all feature sizes and timepoints for no crosslinking case (E) and crosslinked case (F). Scale bars 500 μm.

FIGS. 20A-20H demonstrate that human skin models express expected epidermal proteins. Scale bars 100 micron. (FIGS. 20A-20D) Telocollagen-fibrin skin models. (FIGS. 20E-20H) EDC-crosslinked telocollagen-fibrin skin models.

FIGS. 21A-21G demonstrate that diabetic keratinocytes can be grown on stamped telocollagen and fibrin and stamped, EDC-crosslinked telocollagen and fibrin, cultured for 14 days. (FIG. 21A) Diabetic keratinocytes grown on stamped telocollagen and fibrin gel, no crosslinking. Stamp feature size was 400 μm. (FIGS. 21B-21E) Diabetic keratinocytes grown on stamped, EDC-crosslinked telocollagen and fibrin gel. The stamp sizes were 200 μm (FIG. 21B), 300 μm (FIG. 21C), 400 μm (FIG. 21D), and 500 μm (FIG. 21E). (FIG. 21F) Comparison of the interdigitation index values for diabetic keratinocytes cultured on 400 μm stamps on telocollagen and fibrin gels with no crosslinking. p=0.003 from a two-tailed t-test. (FIG. 21G) Comparison of the interdigitation index values for diabetic and neonatal keratinocytes cultured on stamped and EDC crosslinked telocollagen and fibrin gels. No significant difference was observed between samples. Scale bars 500 μm.

FIGS. 22A-22H demonstrate that human skin models with diabetic keratinocytes express expected epidermal proteins. Scale bars 100 μm.

FIG. 23 depicts a graph demonstrating that stamped models develop epidermal barrier. Data are normalized to PBS control.

FIGS. 24A-24D demonstrate silicone stamps for generating rete pegs in skin models. Stamps used here have features that are (FIG. 24A) 200 μm tall, (FIG. 24B) 300 μm tall, (FIG. 24C) 400 μm tall, and (FIG. 24D) 500 μm tall.

FIGS. 25A-25B demonstrate that telocollagen outperforms atelocollagen in retaining stamped features after four days of culturing with keratinocytes. The images in the left column are from samples fixed at day 0, and the images in the right column are from samples fixed at day 4. (FIG. 25A), 7.8 mg/mL telocollagen and 4.6 mg/mL fibrin. (FIG. 25B), 7.8 mg/mL atelocollagen and 4.6 mg/mL fibrin. Scale bars represent 500 μm.

FIGS. 26A-26E demonstrate that human skin models with telocollagen-fibrin gels stamped with 400-500 μm features show degraded but lasting features over two weeks of culture, while stamps with smaller features do not have a significant difference from flat controls with no stamp. All gels have a composition of 7.8 mg/mL telocollagen and 4.6 mg/mL fibrin. (FIG. 26A) Representative images of samples stamped with 200 μm features at days 0, 4, 7, and 14. (FIG. 26B) Representative images of samples stamped with 300 μm features at days 0, 4, 7, and 14. (FIG. 26C) Representative images of samples stamped with 400 μm features at days 0, 4, 7, and 14. (FIG. 26D) Representative images of samples stamped with 500 μm features at days 0, 4, 7, and 14. (FIG. 26E) Interdigitation index, defined as the length of the interface between the epidermis and dermis divided by the straight-line distance, is plotted at the last timepoint, 14 days. Scale bars represent 500 μm. * p<0.05, *** p<0.001.

FIGS. 27A-27D demonstrate that 7.8 mg/mL telocollagen and 4.6 mg/mL fibrin skin models without keratinocytes retain features up to 14 days of culture. (FIG. 27A) Human skin model stamped with 200 μm features after 14 days of culture with fibroblasts. (FIG. 27B) Human skin model stamped with 300 μm features after 14 days of culture with fibroblasts. (FIG. 27C) Human skin model stamped with 400 μm features after 14 days of culture with fibroblasts. (FIG. 27D) Human skin model stamped with 500 μm features after 14 days of culture with fibroblasts. Scale bars 500 μm.

FIGS. 28A-28E demonstrate that human skin models made from EDC-crosslinked telocollagen-fibrin gels do not show appreciable degradation patterns present in the samples that were not crosslinked. All gels have a composition of 7.8 mg/mL telocollagen and 4.6 mg/mL fibrin. (FIG. 28A) Representative images of samples stamped with 200 μm features at days 0, 4, 7, and 14. (FIG. 28B) Representative images of samples stamped with 300 μm features at days 0, 4, 7, and 14. (FIG. 28C) Representative images of samples stamped with 400 μm features at days 0, 4, 7, and 14. (FIG. 28D) Representative images of samples stamped with 500 μm features at days 0, 4, 7, and 14. (FIG. 28E) Interdigitation index, defined as the length of the interface between the epidermis and dermis divided by the straight-line distance, is plotted for the final timepoint, 14 days.

### DETAILED DESCRIPTION

Described herein are methods for preparing or providing a multi-layer, e.g., 2 layer, cell culture where the interface or boundary between the 2 layers comprises microfeatures such as rete pegs. The method comprises preparing a first pre-gel layer, stamping it with a stamp to imprint the microfeatures onto the pre-gel layer, gelling the first layer, and then culturing a second layer in contact with the first layer. Further described herein are multi-layer cell cultures comprising such microfeatures and/or made according to the methods described herein.

In one aspect of any of the embodiments, described herein is a multi-layer 3-D culture comprising at least: a first layer comprising a first type of cell in a gel; a second layer comprising a second type of cell; a boundary between the first and second layers comprising one or more microfeatures. In some embodiments of any of the aspects, the layers do not comprise vasculature.

As used herein, a “cell culture” refers to an in vitro population of cells having a population of metabolically active cells. The number of these cells can be roughly stable over a period of at least 3 days or can grow. As used herein, “culturing” refers to continuing the viability of a cell or population of cells. In some embodiments of any of the aspects, the phenotype, morphology, number, or differentiation status of the cultured cells can change over time. Conditions suitable for cell culture for different cell types are well known in the art and cell culture media for various cell types is readily available. Exemplary media and conditions are provided elsewhere herein.

As used herein, a “3-D cell culture” or “3-D culture” refers to a cell culture in which the cells extend into 3 dimensions, e.g., the cell culture is not a monolayer but has a depth of more than 1 cell. The 3-D cultures described herein are multi-layer cultures, e.g., two-layer 3-D cultures. As used herein, “layer” refers to region of an entity (e.g, a 3-D culture) that is physically distinguishable from the adjacent regions. Preferably, layers are arranged along an axis, such that at the plane defined by most points on one axis of the culture (typically the x-axis) the culture comprises the same physical characteristics throughout the plane.

As used herein, “microfeature” refers to a three-dimensional shape or feature connected to a surface or plane, such that the shape or feature extends into the the third dimension not occupied by the surface or plane. Furthermore, the microfeature is “micro” in that the feature has at least one dimension (height, width, or depth) which is not larger than 1 mm (e.g., is is “microscale). In some embodiments of any of the aspects, the microfeature has at least two dimensions (height, width, and/or depth) which is not larger than 1 mm (e.g., is is “microscale). It should be noted that in reference to a microfeature, “surface” is understood to have its mathematical meaning, e.g., a surface is a generalization of plane which can have curvature. Accordingly, a plane is a species of a surface. In some embodiments of any of the aspects, the surface or plane is a face of a stamp. In some embodiments of any of the aspects, the surface or plane is a boundary or interface between two layers, e.g, layers of a cell culture.

The height of a microfeature is designated as the axis that extends perpendicularly from the surface or plane of a face or boundary of a layer. The width and length of a microfeature are designated as the dimensions which are parallel to the surface or place of a face or boundary of a layer. In some embodiments of any of the aspects, each microfeature on the face of a stamp extends 1 μm to 1 mm from the face of the stamp. In some embodiments of any of the aspects, each microfeature on the face of a stamp extends 100 μm to 400 μm from the face of the stamp. In some embodiments of any of the aspects, each microfeature on the face of a stamp extends at least 100 μm from the face of the stamp. In some embodiments of any of the aspects, each microfeature on the face of a stamp extends at least 200 μm from the face of the stamp. In some embodiments of any of the aspects, each microfeature on the face of a stamp extends at least 300 μm from the face of the stamp. In some embodiments of any of the aspects, each microfeature on the face of a stamp extends at least 400 μm from the face of the stamp. In some embodiments of any of the aspects, each microfeature on the face of a stamp extends 300-500 μm from the face of the stamp. In some embodiments of any of the aspects, each microfeature on the face of a stamp extends 400-500 μm from the face of the stamp. In some embodiments of any of the aspects, each microfeature on the face of a stamp extends 300 μm to 1 mm from the face of the stamp. In some embodiments of any of the aspects, each microfeature on the face of a stamp extends 400 μm to 1 mm from the face of the stamp.

In some embodiments of any of the aspects, each microfeature extends 1 μm to 1 mm from the surface or plane that defines a boundary of a layer. In some embodiments of any of the aspects, each microfeature on the face of a stamp extends 100 μm to 400 μm from the surface or plane that defines a boundary of a layer. In some embodiments of any of the aspects, each microfeature on the face of a stamp extends at least 100 μm from the surface or plane that defines a boundary of a layer. In some embodiments of any of the aspects, each microfeature on the face of a stamp extends at least 200 μm from the surface or plane that defines a boundary of a layer. In some embodiments of any of the aspects, each microfeature on the face of a stamp extends at least 300 μm from the surface or plane that defines a boundary of a layer. In some embodiments of any of the aspects, each microfeature on the face of a stamp extends at least 400 μm from the surface or plane that defines a boundary of a layer. In some embodiments of any of the aspects, each microfeature on the face of a stamp extends 300-500 μm from the surface or plane that defines a boundary of a layer. In some embodiments of any of the aspects, each microfeature on the face of a stamp extends 400-500 μm from the surface or plane that defines a boundary of a layer. In some embodiments of any of the aspects, each microfeature on the face of a stamp extends 300 μm to 1 mm from the surface or plane that defines a boundary of a layer. In some embodiments of any of the aspects, each microfeature on the face of a stamp extends 400 μm to 1 mm from the surface or plane that defines a boundary of a layer.

In some embodiments of any of the aspects, each microfeature has a width and/or length of at least 100 μm. In some embodiments of any of the aspects, each microfeature has a width and/or length of at least 200 μm. In some embodiments of any of the aspects, each microfeature has a width and/or length of at least 300 μm. In some embodiments of any of the aspects, each microfeature has a width and/or length of at least 400 μm. In some embodiments of any of the aspects, each microfeature has a width and/or length of 300-500 μm. In some embodiments of any of the aspects, each microfeature has a width and/or length of 400-500 μm. In some embodiments of any of the aspects, each microfeature has a height which is no more than 5× the narrowest dimension of the microfeature.

A microfeature can form a shape on a face of a stamp (or on the surface of a cell culture) that is a polyhedron, a hexahedron, a cube, a ridge, a wall, a shape created by introducing a curve or angle into any of the preceding shapes, or a shape created by rounding one or more of the edges of one of the preceding shapes. In some embodiments of any of the aspects, a microfeature can assume the shape and size, or average shape and size of a rete peg. A rete peg is a microfeature found in the naturally-ocurring dermis-epidermis boundary. The size and shapes of naturally-occuring rete pegs are well known in the art, see, e.g, Wu et al. Cell Tissues Organs 2013 197:239-248 and Huzaira et al. Journal of Investigative Dermatology 2001 116:846-852; which are incorporated by reference herein it their entireties. In some embodiments of any of the aspects, a microfeature can be the size and/or shape of a microfeature in diseased tissue. For example, in psoriasis the microfeatures at the dermis/epidermis boundary are elongated (taller) and in solar lentigo the microfeatures at the dermis/epidermis boundary are flattened. Such aberrant microfeature topology in diseased tissues is known in the art, e.g., Murphy et al. Clinics in Dermatology 2007 25:524-528 and Shin et al. Clinical and Experimental Dermatology 2015 40:489-494, each of which is incorporated by reference herein in its entirety.

Accordingly, in one aspect of any of the embodiments, described herein is a method comprising a) applying at least one face of a stamp onto at least one surface of a pre-gel comprising a first type of cell, wherein the at least one face of the stamp comprises one or more microfeatures; b) maintaining contact of the at least one face of the stamp with the at least one surface of the pre-gel while the pre-gel forms a gel; c) separating the at least one face of the stamp and the at least one surface of the gel formed in step b) and thereby providing a first layer comprising a gel comprising at least one stamped surface; and d) contacting the at least one stamped surface of the gel resulting from step c) with at least a second type of cell to provide a second layer comprising at least the second type of cell, whereby the method provides a two layer 3-D culture.

Described herein are methods and compositions relating to a two layer 3-D culture. The two layers of the 3-D culture are distinct in that each comprises at least one type of cell not found in the other layer, e.g., at least at the beginning of culture as during culture individual cells may migrate between layers. During culture, the two layers will remain distinct in that the most common cell type in each layer will be different from the most common cell type in the other layer.

While each layer comprises at least one cell type, each layer can comprise one or more further cell types. The further cell types can be unique to the layer or common to both layers.

It is contemplated herein that the first and second cell types can be any pair (or more complex combination) of cell types which are found in vivo on opposing sides of a boundary comprising microfeatures. Illustrative examples of such pairs or combinations of cell types are provided in Table 1. In some embodiments of any of the aspects, the at least a first type of cell and the at least a second type of cell comprise or are a combination selected from Table 1.

TABLE 1

| Exemplary cells comprised | Exemplary cells in the |
|---|---|
| by the pre-gel, e.g., the | second layer, e.g, that at |
| at least first type of cell | least second type of cell |
| Fibroblasts | keratinocytes |
| Fibroblasts | Keratinocytes + melanocytes |
| Fibroblasts | Melanocytes |
| Fibroblasts + endothelial cells | Keratinocytes |
| Fibroblasts + endothelial cells | Keratinocytes + melanocytes |
| Fibroblasts + immune cells | Keratinocytes/keratinocytes + |
| | melanocytes |
| Fibroblasts | Induced pluripotent stem cells |
| Induced pluripotent stem cells | Induced pluripotent stem cells |
| Fibroblasts | Cells differentiated from Induced |
| | pluripotent stem cells |
| Cells differentiated from Induced | Cells differentiated from Induced |
| pluripotent stem cells | pluripotent stem cells |
| Fibroblasts | Hepatocytes |
| Fibroblasts | Intestinal stem cells |
| Fibroblasts | Intestinal cells |
| Fibroblasts | Tumor cells (e.g., melanoma, liver |
| | cancer, or intestinal cancer cells) |
| Fibroblasts from psoriasis patient | Keratinocytes from psoriasis patient |
| Fibroblasts from diabetic patient | Keratinocytes from diabetic patient |

In some embodiments of any of the aspects, the first type of cell is fibroblasts and the second type of cell is keratinocytes.

In some embodiments of any of the aspects, the first type of cell is fibroblasts and the second type of cell is keratinocytes and the first layer further comprises melanocytes and/or iPSCs. In some embodiments of any of the aspects, the first type of cell is fibroblasts and the second type of cell is keratinocytes and the second layer further comprises immune cells, hair follicle cells, and/or iPSCs.

In some embodiments of any of the aspects, one or both of the layers initially comprise stem cells, progenitor cells, iPSCs, or the like. In some embodiments of any of the aspects, one or both of the layers initially comprise stem cells, progenitor cells, iPSCs, or the like and the cells differentiate or are differentiated during the culturing step(s).

In some embodiments of any of the aspects, one or both of the layers initially further comprise stem cells, progenitor cells, iPSCs, or the like. In some embodiments of any of the aspects, one or both of the layers initially further comprise stem cells, progenitor cells, iPSCs, or the like and the cells differentiate or are differentiated during the culturing step(s).

In some embodiments of any of the aspects, the at least a first type of cell comprises or is fibroblasts; fibroblasts and immune cells; iPSCs; cells differentiated from iPSCs; fibroblasts and endothelial cells; or fibroblasts obtained from a diseased subject (e.g, a subject with psoriasis or diabetes).

In some embodiments of any of the aspects, the at least a second type of cell comprises or is keratinocytes; keratinocytes and melanocytes; melanocytes; iPSCs; cells differentiated from iPSCs; keratinocytes obtained from a diseased subject (e.g, a subject with psoriasis or diabetes); intestinal stem cells; intestinal epithelial cells; hepatocytes; or tumor cells.

In some embodiments of any of the aspects, one or both of the layers further comprise an organioid comprising one or more cell types. In some embodiments of any of the aspects, one or both of the layers initially further comprise an organioid comprising one or more cell types.

The one or more cell types that may be included in the methods or compositions described herein can comprise any mammalian cell type selected from cells that make up the mammalian body, including germ cells, somatic cells, and stem cells. The term “germ cells” refers to any line of cells that give rise to gametes (eggs and sperm). The term “somatic cells” refers to any biological cells forming the body of a multicellular organism; any cell other than a gamete, germ cell, gametocyte or undifferentiated stem cell. Examples of somatic cells include fibroblasts, chondrocytes, osteoblasts, tendon cells, mast cells, wandering cells, immune cells, pericytes, inflammatory cells, endothelial cells, myocytes (cardiac, skeletal and smooth muscle cells), adipocytes (i.e., lipocytes or fat cells), parenchyma cells (neurons and glial cells, nephron cells, hepatocytes, pancreatic cells, lung parenchyma cells) and non-parenchymal cells (e.g., sinusoidal hepatic endothelial cells, Kupffer cells and hepatic stellate cells). The term “stem cells” refers to cells that have the ability to divide for indefinite periods and to give rise to virtually all of the tissues of the mammalian body, including specialized cells. The stem cells include pluripotent cells, which upon undergoing further specialization become multipotent progenitor cells that can give rise to functional or somatic cells. Examples of stem and progenitor cells include hematopoietic stem cells (adult stem cells; i.e., hemocytoblasts) from the bone marrow that give rise to red blood cells, white blood cells, and platelets; mesenchymal stem cells (adult stem cells) from the bone marrow that give rise to stromal cells, fat cells, and types of bone cells; epithelial stem cells (progenitor cells) that give rise to the various types of skin cells; neural stem cells and neural progenitor cells that give rise to neuronal and glial cells; and muscle satellite cells (progenitor cells) that contribute to differentiated muscle tissue.

In some embodiments of any of the aspects, the cells (e.g., the first and/or second types of cells) are mammalian cells. In some embodiments of any of the aspects, the cells (e.g., the first and/or second types of cells) are murine cells. In some embodiments of any of the aspects, the cells (e.g., the first and/or second types of cells) are primate cells. In some embodiments of any of the aspects, the cells (e.g., the first and/or second types of cells) are human cells. In some embodiments of any of the aspects, the cells (e.g., the first and/or second types of cells) are obtained from or derived from the same species of organism. In some embodiments of any of the aspects, the cells (e.g., the first and/or second types of cells) are obtained from or derived from different species.

In some embodiments of any of the aspects, one or more of the types of cells (e.g., the first and/or second types of cells) are primary cells. In some embodiments of any of the aspects, one or more of the types of cells (e.g., the first and/or second types of cells) are cell lines. In some embodiments of any of the aspects, one or more of the types of cells (e.g., the first and/or second types of cells) are cells differentiated from stem cells, progenitor cells, iPSCs, or the like in vitro.

In some embodiments of any of the aspects, one or more of the types of cells (e.g., the first and/or second types of cells) are obtained from or derived from diseased primary cells or a subject having a disease affecting the first and/or second type of cells. For example, in some embodiments the two-layer 3D culture can model a skin disease such as psoriasis and the first and/or second type of cell can comprise cells obtained from or derived from a subject having psoriasis.

In some embodiments of any of the aspects, one or more of the types of cells (e.g., the first and/or second types of cells) can be genetically modified or engineered to express a reporter construct. The function of the reporter construct is to produce a detectable signal when an certain cellular activity or state occurs, e.g, wound healing, inflammatory process, or the like. In some embodiments of any of the aspects, a reporter construct can be used to quantify the concentration, strength, or activity of the cellular activity or state. In some embodiments of any of the aspects, the reporter component comprises a reporter gene, e.g, a gene expressing a detectable signal or label.

Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). Detectable labels can include, but are not limited to radioisotopes, bioluminescent compounds, chromophores, antibodies, chemiluminescent compounds, fluorescent compounds, metal chelates, and enzymes. In some embodiments of any of the aspects, the detectable label or signal is a fluorescent compound, e.g, a fluorescent dye molecule or fluorophore. In some embodiments of any of the aspects, the detectable label or signal is a rabiolabel. In some embodiments of any of the aspects, the detectable label or signal is a chemluminescent compound. In some embodiments of any of the aspects, the detectable label or signal is a enzymatic label, e.g., a enzyme that can produce a chemiluminescent signal, a color signal, or a fluorescent signal. In some embodiments of any of the aspects, a detectable label can be a spectral colorimetric label including, but not limited to colloidal gold or colored glass or plastic (e.g., polystyrene, polypropylene, and latex) beads.

A reporter gene encoding any fluorescent protein can be applicable in the technology described herein. The fluorescent protein includes, but is not limited to, for example, GFP, mCherry, Venus, and Cerulean. Examples of genes encoding fluorescent proteins that can be used in accordance with the compositions and methods described herein include, without limitation, those proteins provided in U.S. Patent Application No. 2012/0003630 (see Table 59), incorporated herein by reference.

Similarly, a reporter gene encoding any enzyme can be applicable as well. Enzymes that produce colored substrates (“colorimetric enzymes”) can also be used for visualization and/or quantification. Enzymatic products can be quantified using spectrophotometers or other instruments that can take absorbance measurements including plate readers. Examples of genes encoding colorimetric enzymes that can be used in accordance with the compositions and methods described herein include, without limitation, lacZ alpha fragment, lacZ (encoding beta-galactosidase, full-length), and xylE. An enzyme (e.g., glucose oxidase) can also change the conductivity of a reaction volume, permitting an electrical or electronic readout (Malitesta et al., Anal Chem 1990, 62, 2735-2740). In another example, a nuclease enzyme can cleave a nucleic acid sequence such that an electronic and optical signal is generated. In yet another example, an enzyme can separate a fluorescence resonance energy transfer (FRET) or quenching pair to induce a change in fluorescence.

A reporter gene encoding any antigen for which a specific antibody is available or can be made can also be applicable. By way of example only, as antigens are expressed by the reporter gene, the antigens bind to an electrode coated with complementary antibodies, which produces an electronic signal. Conversely, a reporter gene can encode an antibody, which when expressed, binds to an electrode coated with the complementary antigen. For non-limiting examples of reporter genes, see Reporter Genes: A Practical Guide, D. Anson (Ed.), 2007, Humana Press, the contents of which are incorporated by reference for examples on reporter genes.

A reporter gene encoding luciferases can also be used in the technology described herein. Luciferases produce luminescence, which can be readily quantified using a plate reader or luminescence counter. Examples of genes encoding luciferases for that can be used in accordance with the compositions and methods described herein include, without limitation, dmMyD88-linker-Rluc, dmMyD88-linker-Rluc-linker-PEST191, and firefly luciferase (from Photinus pyralis).

In one embodiment, the reporter component comprises a catalytic nucleic acid including, but not limited to, a ribozyme, an RNA-cleaving deoxyribozyme, a group I ribozyme, RNase P, a Hepatitis delta ribozyme, and DNA-zymes. The use of catalytic nucleic acid as reporters is described in WO1996027026. In one embodiment, the reporter component comprises a fluorophore, a metabolite, or protein, wherein the fluorophore, metabolite, or protein can couple to a nucleic acid to produce a change in fluorescence. For example, RNA-fluorophore complexes have been reported and can be used in the compositions and methods described herein (see, e.g., Paige et al., Science 2011, 333, 642-646). RNA binding to metabolites or proteins can also lead to a change in fluorescence (see, e.g., Strack et al., Nature Protocols 2014, in press). In one embodiment, the nucleic acid can be the analyte. In another embodiment, the nucleic acid can be transcribed due to the detection of an analyte.

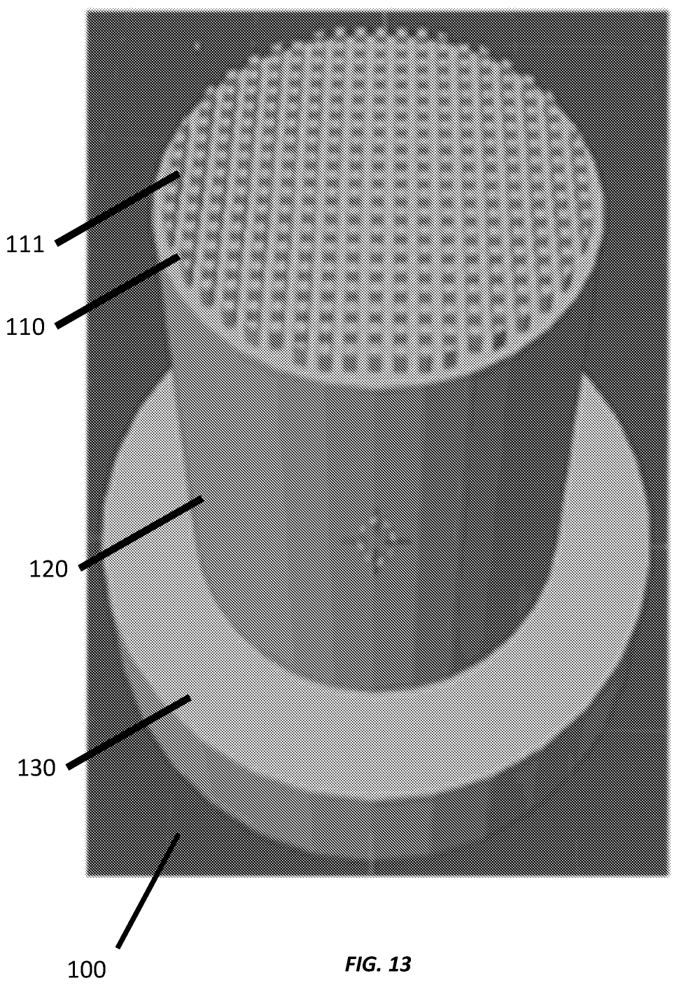

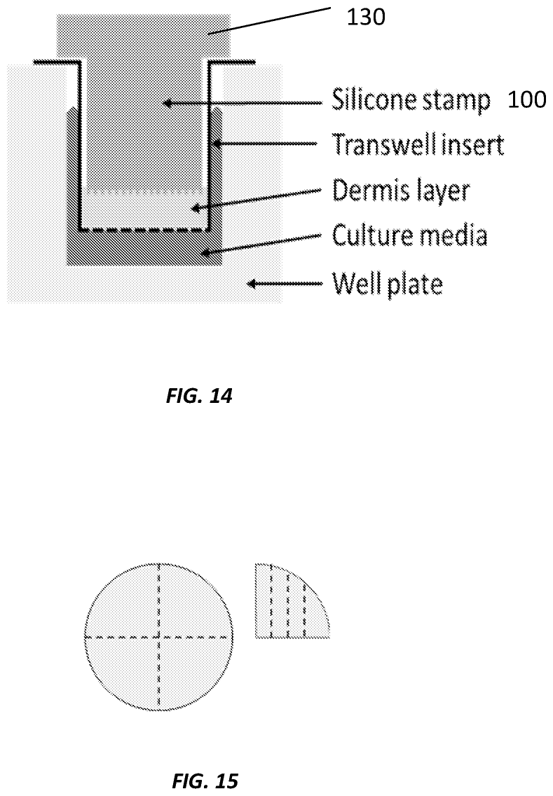

As used herein, “stamp” refers to a device made of a solid or amorphous solid material that can be applied to pre-gel surface. A non-limiting illustrative embodiment is depicted in FIG. 13. The stamp 100 can have one or more faces 101 that are applied to, e.g., that contact, the pre-gel surface. Each face 101 of a stamp 100 comprises one or more microfeatures 111, where the face of the stamp is considered to be the reference surface or plane. In some embodiments of any of the aspects, the stamp can comprise a face 110 and a body or handle portion 120. The body or handle portion 120 extends from the face 110 and can be sized to be grasped by a human or grasped by/attached to a machine. In some embodiments of any of the aspects, the stamp can comprise a rest portion 130. The rest portion 130 is sized/configured to rest on the top of a well, dish, Transwell insert, or other culture container, such that the face 110 can be maintained in contact with the pre-gel. An exemplary embodiment is depicted in FIG. 14. In such embodiments, the length of the body or handle portion 120 will be determined by the depth of the well, dish, Transwell inert, or other culture container and the desired depth of the pre-gel. One of ordinary skill in the art can readily design a stamp having dimensions suitable for the well, dish, Transwell inert, or other culture container and pre-gel depth a user desires to utilize.

In embodiments comprising multiple faces on a stamp, the faces can be identical or different in size and microfeature composition. In embodiments comprising multiple faces on a stamp, the stamp can be configured to contact one or more pre-gels at the same time. For example, a stamp could have multiple faces which can be applied simultaneously to one large pre-gel, thereby providing stamping of microfeatures onto different zones of the pre-gel. Or alternatively, a stamp could have multiple faces which can be applied simultaneously to different faces or sides of a pre-gel, thereby providing stamping of a 3-dimensional pre-gel instead of merely on atop face of the pre-gel. Or alternatively, a stamp could have multiple faces which can be applied simultaneously to multiple pre-gels (e.g., in different wells of a multi-well plate), thereby providing stamping of microfeatures onto different pre-gels. Or alternatively, a stamp could have multiple faces which can be applied sequentially to pre-gels.

In some embodiments of any of the aspects, the stamp or at least the face(s) of the stamp is made of biocompatible material. A biocompatible material is a material which does not have toxic or injurious effects on biological functions. A biocompatible material can be made of materials such as metals, ceramics, polymers or a combination of any of these. Biocompatible materials can be a polymer, a ceramic, metal, ceramics, polymers, hydrogels or a combination of any of these materials. Biocompatible materials include, but are not limited to an oxide, a phosphate, a carbonate, a nitride or a carbonitride, e.g, tantalum oxide, aluminum oxide, iridium oxide, zirconium oxide or titanium oxide, zirconia, alumina, or calcium phosphate. Biocompatible polymers include natural or synthetic polymers. Examples of biocompatible polymers include, but are not limited to, collagen, poly(alpha esters) such as poly(lactate acid), poly(glycolic acid), polyorthoesters and polyanhydrides and their copolymers, polyglycolic acid and polyglactin, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, polyglactin, or copolymers or physical blends of these materials.

In some embodiments, a biocompatible material for a stamp can be rubber, silicone rubber (e.g, ECOFLEX 00-50), or polydimethylsiloxane.

A stamp can be made by any method known in the art, e.g., 3-D printing, casting in a mold, casting in a 3-D printed mold, photolithography, solvent casting, compression molding, filament drawing, meshing, leaching, weaving, coating. In some embodiments of any of the aspects, the stamp is produced or provided by 3-D printing. In some embodiments of any of the aspects, the stamp is produced or provided by casting the stamp in a 3-D printed mold. In some embodiments of any of the aspects, at least the face of the stamp comprising at least one microfeature is produced or provided by 3-D printing. In some embodiments of any of the aspects, at least the face of the stamp comprising at least one microfeature is produced or provided by casting the stamp in a 3-D printed mold.

The methods described herein relate to contacting a pre-gel with a stamp and then permitting or causing the pre-gel to form a gel.

As used herein, the term “gel” refers to the state of matter between liquid and solid. As such, a “gel” has some of the properties of a liquid (i.e., the shape is resilient and deformable) and some of the properties of a solid (i.e., the shape is discrete enough to maintain three dimensions on a two dimensional surface.). A non-limiting example of a gel is a hydrogel. A hydrogel is a substance that is formed when an organic polymer (natural or synthetic) is crosslinked via covalent, ionic, or hydrogen bonds to create a three-dimensional open-lattice structure which entraps water molecules to form a gel. Gels are well known in the art and non-limiting examples are provided below herein.

As used herein, the term “pre-gel” refers to a composition comprising one or more components of a gel, but which is a liquid. In some embodiments of any of the aspects, the pre-gel comprises all of the components of a gel but is not yet cross-linked or polymerized.

In some embodiments of any of the aspects, the pre-gel comprises collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; Alginate; a modified alginate (e.g, RGD-modified alginate, or acrylate modified alginate (for photopolymerization or in combination with PEGs listed elsewhere herein); Hyaluronic acid; Agarose; Poly(L-lactic acid); poly(vinyl alcohol); poly(hydroxyl-ethyl methacrylate); EDC (1-ethyl-3-(3-dimethylaminopropyl)carbodiimide hydrochloride) as crosslinker for biological gels such as collagen; or a combination of any of the foregoing. In some embodiments of any of the aspects, the pre-gel comprises collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing. In some embodiments of any of the aspects, the pre-gel comprises a) at least one of collagen, atellocollagen, telocollagen, collagen methacrylate, polyethylene glycol (PEG), PEG-DA, fibrin, fibrinogen, gelatin, agarose, thrombin; and b) PBS, NaOH, and Phenolphthalein. In some embodiments of any of the aspects, the pre-gel comprises a) at least one of collagen, atellocollagen, telocollagen, collagen methacrylate, polyethylene glycol (PEG), PEG-DA, fibrin, fibrinogen, gelatin, agarose, thrombin; and b) PBS and Phenolphthalein. In some embodiments of any of the aspects, the pre-gel comprises a) at least one of collagen, atellocollagen, telocollagen, collagen methacrylate, polyethylene glycol (PEG), PEG-DA, fibrin, fibrinogen, gelatin, agarose, thrombin; and b) PBS and Phenolphthalein. In some embodiments of any of the aspects, the pre-gel comprises a) at least one of collagen, atellocollagen, telocollagen, collagen methacrylate, polyethylene glycol (PEG), PEG-DA, fibrin, fibrinogen, gelatin, agarose, thrombin; and b) PBS. PBS can be provided as a buffer, phenolphthalein can be provided as a pH indicator, and NaOH can be provided as a neutralizer.

In some embodiments of any of the aspects, the pre-gel comprises telocollagen. In some embodiments of any of the aspects, the pre-gel comprises telocollagen and fibrinogen.

In some embodiments of any of the aspects, the pre-gel does not comprise atelocollagen. In some embodiments of any of the aspects, the pre-gel comprises telocollagen and does not comprise atelocollagen. In some embodiments of any of the aspects, the pre-gel comprises telocollagen and fibrinogen, and does not comprise atelocollagen.

In some embodiments of any of the aspects, the collagen is LifeInk™ collagen (e.g, LifeInk 200 or LifeInk 240, e.g, 35 mg/mL from Advanced Biomatrix San Diego CA; Cat. No. 5278). In some embodiments of any of the aspects, the agarose is low-melting point agarose (e.g., Ultrapure Low Melting Point Agarose from Invitrogen Waltham MA; Cat No. 16520050). In some embodiments of any of the aspects, the telocollagen is bovine telocollagen, e.g., 3 mg/mL from Advanced Biomatrix San Diego CA; Cat. No. 5026). In some embodiments of any of the aspects, the atelocollagen is bovine atelocollagen, e.g., 3 mg/mL from Advanced Biomatrix San Diego CA; Cat. No. 5005). In some embodiments of any of the aspects, the gelatin is Type A gelatin, and/or from porcine skin, and/or having a gel strength of 300 (e.g., Cat No. G2500; Sigma-Aldrich St. Louis MO). In some embodiments of any of the aspects, the fibrinogen is bovine, e.g. Cat. No. 341573 (EMD Millipore, Burlington, MA).

In some embodiments of any of the aspects, the PEG in a pre-gel can comprise one or more of: PEG-diacrylate; PEG-norbornene, 4-arm+PEG-dithiol; Lithium phenyl-2,4,6-trimethylbenzoylphosphinate (photoinitiator); Irgacure 2959 2-Hydroxy-4′-(2-hydroxyethoxy)-2-methylpropiophenone (photoinitiator); Eosin Y (photoinitiator); and Triethanolamine (coinitiator if Eosin Y and PEG-DA are used together). In some embodiments of any of the aspects, the pre-gel can comprise collagen methacrylate and/or gelatin methacrylate and PEG comprising one or more of: PEG-diacrylate; PEG-norbornene, 4-arm+PEG-dithiol; Lithium phenyl-2,4,6-trimethylbenzoylphosphinate (photoinitiator); Irgacure 2959 2-Hydroxy-4′-(2-hydroxyethoxy)-2-methylpropiophenone (photoinitiator); Eosin Y (photoinitiator); and Triethanolamine (coinitiator if Eosin Y and PEG-DA are used together).

In some embodiments of any of the aspects, the pre-gel comprises more than 6 mg/mL of collagen, atellocollagen, and/or telocollagen collectively. In some embodiments of any of the aspects, the pre-gel comprises more than 6 mg/mL of each of collagen, atellocollagen, or telocollagen. In some embodiments of any of the aspects, the pre-gel comprises more than 8 mg/mL of collagen, atellocollagen, and/or telocollagen collectively. In some embodiments of any of the aspects, the pre-gel comprises more than 8 mg/mL of each of collagen, atellocollagen, or telocollagen.

Tables 2 and 3 provide exemplary pre-gel formulations. In some embodiments of any of the aspects, the pre-gel comprises any one of the formulations of Table 2 or Table 3. In some embodiments of any of the aspects, the pre-gel comprises any one of the formulations of Table 2 or Table 3 and at least a first type of cell. In some embodiments of any of the aspects, the pre-gel comprises any one of the formulations of Table 2 or Table 3 and fibroblasts.

TABLE 2

| | Formulation | | | | | | | | | | | | | | | | | |
|---|---|---|---|---|---|---|---|---|---|---|---|---|---|---|---|---|---|---|
| Component | 1 | 2 | 3 | 4 | 5 | 6 | 7 | 8 | 9 | 10 | 11 | 12 | 13 | 14 | 15 | 16 | 17 | 18 |
| Telocollagen | x | | | X | | | x | | | x | | | x | | | X | | |
| Telocollagen | | x | | | X | | | x | | | x | | | x | | | X | |
| up to 10 mg/mL | | | | | | | | | | | | | | | | | | |
| Telocollagen | | | x | | | X | | | x | | | x | | | x | | | X |
| 4-9 mg/mL | | | | | | | | | | | | | | | | | | |
| atelocollagen | | | | | | | | | | | | | | | | | | |
| atelocollagen | | | | | | | | | | | | | | | | | | |
| up to 10 mg/mL | | | | | | | | | | | | | | | | | | |
| atelocollagen | | | | | | | | | | | | | | | | | | |
| 4-9 mg/mL | | | | | | | | | | | | | | | | | | |
| transglutaminase | | | | | | | x | x | x | | | | | | | x | X | x |
| Fibrinogen | | | | | | | | | | x | x | x | | | | x | x | X |
| Fibrinogen | | | | | | | | | | | | | x | x | x | | | |
| 1-10 mg/mL | | | | | | | | | | | | | | | | | | |
| Thrombin | | | | | | | | | | x | x | x | | | | x | x | x |
| Thrombin 0.25 | | | | | | | | | | | | | x | x | x | | | |
| U/mL-2 U/mL | | | | | | | | | | | | | | | | | | |
| collagen | | | | | | | | | | | | | | | | | | |
| Agarose, e.g., | | | | | | | | | | | | | | | | | | |
| 0.25-2% | | | | | | | | | | | | | | | | | | |
| PBS | x | x | x | x | X | x | x | X | x | x | x | x | x | x | X | X | X | X |
| Phenolphthalein | x | x | x | x | x | x | x | x | x | x | x | x | x | x | x | X | X | X |
| NaOH | x | x | x | x | x | x | x | x | x | x | x | x | x | x | x | x | X | X |
| | Formulation | | | | | | | | | | | | | | | | | |
| Component | 19 | 20 | 21 | 22 | 23 | 24 | 25 | 26 | 27 | 28 | 29 | 30 | 31 | 32 | 33 | 34 | 35 | 36 |
| Telocollagen | x | | | | | | | | | | | | X | | | X | | |
| Telocollagen | | x | | | | | | | | | | | | X | | | X | |
| up to 10 mg/mL | | | | | | | | | | | | | | | | | | |
| Telocollagen | | | x | | | | | | | | | | | | X | | | X |
| 4-9 mg/mL | | | | | | | | | | | | | | | | | | |
| atelocollagen | | | | x | | | X | | | X | | | | | | | | |
| atelocollagen | | | | | x | | | x | | | x | | | | | | | |
| up to 10 mg/mL | | | | | | | | | | | | | | | | | | |
| atelocollagen | | | | | | x | | | x | | | x | | | | | | |
| 4-9 mg/mL | | | | | | | | | | | | | | | | | | |
| transglutaminase | x | x | x | | | | | | | | | | x | x | x | x | x | x |
| Fibrinogen | | | | | | | | | | | | | | | | | | |
| Fibrinogen | x | x | x | | | | | | | | | | x | x | x | x | x | X |
| 1-10 mg/mL | | | | | | | | | | | | | | | | | | |
| Thrombin | | | | | | | | | | | | | | | | | | |
| Thrombin 0.25 | x | x | x | | | | | | | | | | x | x | X | x | x | X |
| U/mL-2 U/mL | | | | | | | | | | | | | | | | | | |
| collagen | | | | | | | x | x | x | | | | x | x | x | | | |
| Agarose, e.g., | | | | | | | | | | x | x | x | | | | x | x | X |
| 0.25-2% | | | | | | | | | | | | | | | | | | |
| PBS | x | x | x | x | x | x | x | x | x | x | x | X | x | x | x | x | X | x |
| Phenolphthalein | x | x | x | x | x | x | x | x | x | x | x | x | x | x | x | x | x | X |
| NaOH | x | x | x | x | x | x | x | x | x | x | x | x | x | x | x | x | x | x |
| | Formulation | | | | | | | | | | | | | | | | | |
| Component | 37 | 38 | 39 | 40 | 41 | 42 | 43 | 44 | 45 | 46 | 47 | 48 | 49 | 50 | 51 | 52 | 53 | 54 |
| Telocollagen | x | | | X | | | x | | | x | | | x | | | X | | |
| Telocollagen | | x | | | X | | | x | | | x | | | x | | | X | |
| up to 10 mg/mL | | | | | | | | | | | | | | | | | | |
| Telocollagen | | | x | | | X | | | x | | | x | | | x | | | X |
| 4-9 mg/mL | | | | | | | | | | | | | | | | | | |
| atelocollagen | | | | | | | | | | | | | | | | | | |
| atelocollagen | | | | | | | | | | | | | | | | | | |
| up to 10 mg/mL | | | | | | | | | | | | | | | | | | |
| atelocollagen | | | | | | | | | | | | | | | | | | |
| 4-9 mg/mL | | | | | | | | | | | | | | | | | | |
| transglutaminase | | | | | | | x | x | x | | | | | | | x | X | x |
| Fibrinogen | | | | | | | | | | x | x | x | | | | x | x | X |
| Fibrinogen | | | | | | | | | | | | | x | x | x | | | |
| 1-10 mg/mL | | | | | | | | | | | | | | | | | | |
| Thrombin | | | | | | | | | | x | x | x | | | | x | x | x |
| Thrombin 0.25 | | | | | | | | | | | | | x | x | x | | | |
| U/mL-2 U/mL | | | | | | | | | | | | | | | | | | |
| collagen | x | x | x | x | x | x | x | x | x | x | x | x | x | x | x | x | x | x |
| Agarose, e.g., | | | | | | | | | | | | | | | | | | |
| 0.25-2% | | | | | | | | | | | | | | | | | | |
| PBS | x | x | x | x | X | x | x | X | x | x | x | x | x | x | X | X | X | X |
| Phenolphthalein | x | x | x | x | x | x | x | x | x | x | x | x | x | x | x | X | X | X |
| NaOH | x | x | x | x | x | x | x | x | x | x | x | x | x | x | x | x | X | X |
| | Formulation | | | | | | | | | | | | | | | | | |
| Component | 55 | 56 | 57 | 58 | 59 | 60 | 61 | 62 | 63 | 64 | 65 | 66 | 67 | 68 | 69 | 70 | 71 | 72 |
| Telocollagen | x | | | X | | | x | | | x | | | x | | | X | | |
| Telocollagen | | x | | | X | | | x | | | x | | | x | | | X | |
| up to 10 mg/mL | | | | | | | | | | | | | | | | | | |
| Telocollagen | | | x | | | X | | | x | | | x | | | x | | | X |
| 4-9 mg/mL | | | | | | | | | | | | | | | | | | |
| atelocollagen | | | | | | | | | | | | | | | | | | |
| atelocollagen | | | | | | | | | | | | | | | | | | |
| up to 10 mg/mL | | | | | | | | | | | | | | | | | | |
| atelocollagen | | | | | | | | | | | | | | | | | | |
| 4-9 mg/mL | | | | | | | | | | | | | | | | | | |
| transglutaminase | | | | | | | x | x | x | | | | | | | x | X | x |
| Fibrinogen | | | | | | | | | | x | x | x | | | | x | x | X |
| Fibrinogen | | | | | | | | | | | | | x | x | x | | | |
| 1-10 mg/mL | | | | | | | | | | | | | | | | | | |
| Thrombin | | | | | | | | | | x | x | x | | | | x | x | x |
| Thrombin 0.25 | | | | | | | | | | | | | x | x | x | | | |
| U/mL-2U/mL | | | | | | | | | | | | | | | | | | |
| collagen | | | | | | | | | | | | | | | | | | |
| Agarose, e.g., | x | x | x | x | x | x | x | x | x | x | x | x | x | x | x | x | x | x |
| 0.25-2% | | | | | | | | | | | | | | | | | | |
| PBS | x | x | x | x | X | x | x | X | x | x | x | x | x | x | X | X | X | X |
| Phenolphthalein | x | x | x | x | x | x | x | x | x | x | x | x | x | x | x | X | X | X |
| NaOH | x | x | x | x | x | x | x | x | x | x | x | x | x | x | x | x | X | X |

TABLE 3

| | Formulation | | | | | | | | | | | | | | | |
|---|---|---|---|---|---|---|---|---|---|---|---|---|---|---|---|---|
| Component | 73 | 74 | 75 | 76 | 77 | 78 | 79 | 80 | 81 | 82 | 83 | 84 | 85 | 86 | 87 | 88 |
| Gelatin | x | | x | | x | | x | | X | | x | | x | | X | |
| Gelatin 25-75 | | x | | x | | x | | x | | X | | x | | x | | x |
| mg/mL | | | | | | | | | | | | | | | | |
| Fibrinogen | x | x | | | x | x | | | x | x | | | x | X | | |
| Fibrinogen | | | x | x | | | x | x | | | x | x | | | x | X |
| 1-10 mg/mL | | | | | | | | | | | | | | | | |
| Thrombin | x | x | x | x | | | | | x | x | x | X | | | | |
| Thrombin 0.25 | | | | | x | x | x | x | | | | | x | x | x | X |
| U/mL-2 U/mL | | | | | | | | | | | | | | | | |
| transglutaminase | x | x | x | x | x | x | x | x | x | x | x | x | x | x | x | x |
| collagen | | | | | | | | | x | x | x | x | x | x | x | X |
| Telocollagen | | | | | | | | | | | | | | | | |
| Telocollagen | | | | | | | | | | | | | | | | |
| up to 10 mg/mL | | | | | | | | | | | | | | | | |
| Telocollagen | | | | | | | | | | | | | | | | |
| 4-9 mg/mL | | | | | | | | | | | | | | | | |
| atelocollagen | | | | | | | | | | | | | | | | |
| atelocollagen | | | | | | | | | | | | | | | | |
| up to 10 mg/mL | | | | | | | | | | | | | | | | |
| atelocollagen | | | | | | | | | | | | | | | | |
| 4-9 mg/mL | | | | | | | | | | | | | | | | |
| PBS | x | x | x | x | X | x | x | X | x | x | x | x | x | x | X | X |

| | Formulation | | | | | | | | | | | | | | | |
|---|---|---|---|---|---|---|---|---|---|---|---|---|---|---|---|---|
| Component | 89 | 90 | 91 | 92 | 93 | 94 | 95 | 96 | 97 | 98 | 99 | 100 | 101 | 102 | 103 | 104 |
| Gelatin | x | | x | | x | | x | | X | | x | | x | | X | |
| Gelatin 25-75 | | x | | x | | x | | x | | X | | x | | x | | x |
| mg/mL | | | | | | | | | | | | | | | | |
| Fibrinogen | x | x | | | x | x | | | x | x | | | x | X | | |
| Fibrinogen | | | x | x | | | x | x | | | x | x | | | x | X |
| 1-10 mg/mL | | | | | | | | | | | | | | | | |
| Thrombin | x | x | x | x | | | | | x | x | x | X | | | | |
| Thrombin 0.25 | | | | | x | x | x | x | | | | | x | x | x | X |
| U/mL-2 U/mL | | | | | | | | | | | | | | | | |
| transglutaminase | x | x | x | x | x | x | x | x | x | x | x | x | x | x | x | x |
| collagen | | | | | | | | | | | | | | | | |
| Telocollagen | x | x | x | x | x | x | x | x | | | | | | | | |
| Telocollagen | | | | | | | | | x | x | x | x | x | x | x | x |
| up to 10 mg/mL | | | | | | | | | | | | | | | | |
| Telocollagen | | | | | | | | | | | | | | | | |
| 4-9 mg/mL | | | | | | | | | | | | | | | | |
| atelocollagen | | | | | | | | | | | | | | | | |
| atelocollagen | | | | | | | | | | | | | | | | |
| up to 10 mg/mL | | | | | | | | | | | | | | | | |
| atelocollagen | | | | | | | | | | | | | | | | |
| 4-9 mg/mL | | | | | | | | | | | | | | | | |
| PBS | x | x | x | x | X | x | x | X | x | x | x | x | x | x | X | X |

| | Formulation | | | | | | | | | | | | | | | |
|---|---|---|---|---|---|---|---|---|---|---|---|---|---|---|---|---|
| Component | 105 | 106 | 107 | 108 | 109 | 110 | 111 | 112 | 113 | 114 | 115 | 116 | 117 | 118 | 119 | 120 |
| Gelatin | x | | x | | x | | x | | X | | x | | x | | X | |
| Gelatin 25-75 | | x | | x | | x | | x | | X | | x | | x | | x |
| mg/ml | | | | | | | | | | | | | | | | |
| Fibrinogen | x | x | | | x | x | | | x | x | | | x | X | | |
| Fibrinogen | | | x | x | | | x | x | | | x | x | | | x | X |
| 1-10 mg/mL | | | | | | | | | | | | | | | | |
| Thrombin | x | x | x | x | | | | | x | x | x | X | | | | |
| Thrombin 0.25 | | | | | x | x | x | x | | | | | x | x | x | X |
| U/mL-2 U/mL | | | | | | | | | | | | | | | | |
| transglutaminase | x | x | x | x | x | x | x | x | x | x | x | x | x | x | x | x |
| collagen | | | | | | | | | | | | | | | | |
| Telocollagen | | | | | | | | | | | | | | | | |
| Telocollagen | | | | | | | | | | | | | | | | |
| up to 10 mg/mL | | | | | | | | | | | | | | | | |
| Telocollagen | x | x | x | x | x | x | x | X | | | | | | | | |
| 4-9 mg/mL | | | | | | | | | | | | | | | | |
| atelocollagen | | | | | | | | | x | x | x | x | x | x | x | x |
| atelocollagen | | | | | | | | | | | | | | | | |
| up to 10 mg/mL | | | | | | | | | | | | | | | | |
| atelocollagen | | | | | | | | | | | | | | | | |
| 4-9 mg/mL | | | | | | | | | | | | | | | | |
| PBS | x | x | x | x | X | x | x | x | X | x | x | x | x | x | X | X |
| | Formulation | | | | | | | | | | | | | | | |
| Component | 121 | 122 | 123 | 124 | 125 | 126 | 127 | 128 | 129 | 130 | 131 | 132 | 133 | 134 | 135 | 136 |
| Gelatin | x | | x | | x | | x | | X | | x | | x | | X | |
| Gelatin 25-75 | | x | | x | | x | | x | | X | | x | | x | | x |
| mg/mL | | | | | | | | | | | | | | | | |
| Fibrinogen | x | x | | | x | x | | | x | x | | | x | X | | |
| Fibrinogen | | | x | x | | | x | x | | | x | x | | | x | X |
| 1-10 mg/mL | | | | | | | | | | | | | | | | |
| Thrombin | x | x | x | x | | | | | x | x | x | X | | | | |
| Thrombin 0.25 | | | | | x | x | x | x | | | | | x | x | x | X |
| U/mL-2 U/mL | | | | | | | | | | | | | | | | |
| transglutaminase | x | x | x | x | x | x | x | x | x | x | x | x | x | x | x | x |
| collagen | | | | | | | | | | | | | | | | |
| Telocollagen | | | | | | | | | | | | | | | | |
| Telocollagen | | | | | | | | | | | | | | | | |
| up to 10 mg/mL | | | | | | | | | | | | | | | | |
| Telocollagen | | | | | | | | | | | | | | | | |
| 4-9 mg/mL | | | | | | | | | | | | | | | | |
| atelocollagen | | | | | | | | | | | | | | | | |
| atelocollagen | x | x | x | x | x | x | x | X | | | | | | | | |
| up to 10 mg/mL | | | | | | | | | | | | | | | | |
| atelocollagen | | | | | | | | | x | x | x | x | x | x | x | x |
| 4-9 mg/mL | | | | | | | | | | | | | | | | |
| PBS | x | x | x | x | X | x | x | X | x | x | x | x | x | x | X | X |

In some embodiments of any of the aspects, a pre-gel can further comprise fluorescent material, e.g., fluorescent beads, e.g., Fluoresbrite Microspheres from Polysciences (Warrington, PA). In some embodiments of any of the aspects, a pre-gel can comprise any one of the formulations of Tables 2 and 3 and further comprise fluorescent material, e.g., fluorescent beads, e.g., Fluoresbrite Microspheres from Polysciences (Warrington, PA).

In some embodiments of any of the aspects, the gel or pre-gel has a depth of from about 100 μm to about 10 mm. In some embodiments of any of the aspects, the gel or pre-gel has a depth of from 100 μm to 10 mm. In some embodiments of any of the aspects, the gel or pre-gel has a depth of from about 500 μm to about 4 mm. In some embodiments of any of the aspects, the gel or pre-gel has a depth of from 500 μm to 4 mm. In some embodiments of any of the aspects, the gel or pre-gel has a depth of from about 500 μm to about 2 mm. In some embodiments of any of the aspects, the gel or pre-gel has a depth of from 500 μm to 2 mm.

In some embodiments of any of the aspects, the pre-gel is provided or located in a Transwell insert. In some embodiments of any of the aspects, the pre-gel is provided or located in a cell culture dish or plate, including a multi-well dish or plate. In some embodiments of any of the aspects, the pre-gel is provided or located in a fluidic or microfluidic cell culture device.

As used herein in reference to a gel, pre-gel, or layer, “surface” refers to the boundary or edge of the gel, pre-gel, or layer and is a generalization of plane which can have curvature. The surface of a gel, pre-gel, or layer can have multiple faces, e.g, when the gel, pre-gel, or layer is in a culture container that constrains the gel, pre-gel, or layer into assuming a cuboid, tubular, or cylindrical shape.

In some embodiments of any of the aspects, the at least one face of a stamp is applied to a portion of a surface of the pre-gel. In some embodiments of any of the aspects, the at least one face of a stamp is applied to one face of the surface of the pre-gel. In some embodiments of any of the aspects, the at least one face of a stamp is applied to a portion of one face of the surface of the pre-gel. In some embodiments of any of the aspects, the at least one face of a stamp is applied to an upper surface or upper face of the surface of the pre-gel. In some embodiments of any of the aspects, the at least one face of a stamp is applied to a portion of an upper surface or portion of an upper face of the surface of the pre-gel.

A stamp is applied to a pre-gel surface when the pre-gel surface is contacted with the at least one surface of the stamp with sufficient force and/or contact to cause the pre-gel to be imprinted with the microfeatures of the stamp, e.g., the pre-gel should come in contact with the surface or plane of the face as well as the microfeatures. The force and/or contact should not be so large that the body/handle portion of the stamp becomes inserted in the pre-gel. In some embodiments of any of the aspects, the force and/or contact should not be so large that the pre-gel extends past the surface or place of the face of the stamp to contact the sides of the stamp. In some embodiments of any of the aspects, the force and/or contact should not be so large that the pre-gel extends, visually or substantially, past the surface or place of the face of the stamp to contact the sides of the stamp. The precise degree of force and/or contact necessary will vary depending on the weight of the stamp and the characteristics of the pre-gel. It is well within the ability of one of ordinary skill in the art to determine an appropriate degree of force and/or contact by calculation or observation.

Once the at least one face of the stamp is applied to the at least one surface of the pre-gel, the contact between the face of the stamp and the surface of the pre-gel is maintained while the pre-gel forms a gel. In some embodiments of any of the aspects, the contact is maintained until the pre-gel is at least 50%, at least 60%, at least 70%, at least 80%, at least 85%, at least 90%, at least 95%, at least 98%, or completely gelled. In some embodiments of any of the aspects, the contact is maintained until the pre-gel is at least 80% gelled. In some embodiments of any of the aspects, the contact is maintained until the pre-gel is at least 90% gelled. In some embodiments of any of the aspects, the contact is maintained until the pre-gel is completely gelled.

The time and conditions necessary for gelling will vary depending on the composition of the pre-gel. The requisite conditions necessary for gelling are well known in the art for the gel components described herein and can vary depending on the desired gelation speed and preferred culture conditions for the first type of cells. Merely as illustrative examples, a collagen pre-gel will gell if maintained at 37° C.; a fibrin gel will gell in the presence of thrombin, which polymerizes the fibrin; gelling of a pre-gel comprising PEG can be enhanced or promoted by exposing the pre-gel to LED, UV, violet, or green light (depending on the photoinitiator and polymerization chemistry); a pre-gel comprising collagen methacrylate and PEGDA can be gelled by providing a temperature gelation step at 37° C. and a simultaneously or sequential light gelation step for the PEGDA.

In some embodiments of any of the aspects, during the step of maintaining contact of the at least one face of the stamp with the at least one surface of the pre-gel while the pre-gel forms a gel, the temperature is maintained at from about 30-42° C. In some embodiments of any of the aspects, during the step of maintaining contact of the at least one face of the stamp with the at least one surface of the pre-gel while the pre-gel forms a gel, the temperature is maintained at from 30-42° C. In some embodiments of any of the aspects, during the step of maintaining contact of the at least one face of the stamp with the at least one surface of the pre-gel while the pre-gel forms a gel, the temperature is maintained at from about 35-39° C. In some embodiments of any of the aspects, during the step of maintaining contact of the at least one face of the stamp with the at least one surface of the pre-gel while the pre-gel forms a gel, the temperature is maintained at from 35-39° C. In some embodiments of any of the aspects, during the step of maintaining contact of the at least one face of the stamp with the at least one surface of the pre-gel while the pre-gel forms a gel, the temperature is maintained at about 37° C. In some embodiments of any of the aspects, during the step of maintaining contact of the at least one face of the stamp with the at least one surface of the pre-gel while the pre-gel forms a gel, the temperature is maintained at 37° C.

After the pre-gel has gelled, the at least one face of the stamp and the at least one surface of the gel are separated, thereby providing a gel comprising at least one stamped surface. The separation can be accomplished by lifting or moving the stamp manually or by a machine.

The at least one stamped surface of the gel is then contacted with a second type of cell to provide a two layer 3-D culture. The contacting with a second type of cell can be performed immediately after the separating step, or the gel comprising the first type of cell can be cultured or stored prior to contacting with with a second type of cell.

The second type of cell can be provided in a cell culture medium or fluid. In some embodiments of any of the aspects, the second type of cell can be provided in a pre-gel which is gelled after the gel comprising at least one stamped surface is contacted. The second type of cell can be applied to the at least one stamped surface by pipetting, pouring, or fluidics (including microfluidics).

In some some embodiments of any of the aspects, the methods described herein can further comprise a step e) (or step after the addition of the second cell type, the step comprising culturing the 3-D culture. The culturing of the 3-D culture can comprise culturing the 3-D culture until a) one or both of the layers reaches a desired developmental or differentiation stage and/or b) one or both of the layers reaches a desired depth or cellular density. As discussed elsewhere herein, culturing refers to maintain a cell culture over time and can comprise contacting the culture with appropriate media and/or providing appropriate environmental conditions (such as temperature and humidity). The appropriate conditions and media will vary depending on cell type selected and selection of the appropriate conditions and media is well within the ordinary skill in the art, e.g., utilizing a commercially available media advertised for that cell type. Culturing can in in static or flowing media and comprising changing the media at intervals or continuously.

In some embodiments of any of the aspects, the 3-D culture is cultured until the layer comprising the second cell type forms a layer at least 50 μm in depth (as measured from the point the microfeatures extend most fully into the layer comprising the second cell type. In some embodiments of any of the aspects, the 3-D culture is cultured until the layer comprising the second cell type forms a layer at least 10 μm in depth (as measured from the point the microfeatures extend most fully into the layer comprising the second cell type. In some embodiments of any of the aspects, the 3-D culture is cultured until the layer comprising the second cell type forms a layer at least 150 μm in depth (as measured from the point the microfeatures extend most fully into the layer comprising the second cell type.

In some embodiments of any of the aspects, the second cell type is keratinocytes and the 3-D culture is cultured until the keratinocytes form a stratified epidermis layer, e.g., until a basement membrane has formed.

In some embodiments of any of the aspects, the second cell type is keratinocytes and the 3-D culture is cultured until the keratinocytes form layer at least 50 μm in depth (as measured from the point the microfeatures extend most fully into the layer comprising the keratinocytes. In some embodiments of any of the aspects, the second cell type is keratinocytes and the 3-D culture is cultured until the keratinocytes form layer at least 100 μm in depth (as measured from the point the microfeatures extend most fully into the layer comprising the keratinocytes. In some embodiments of any of the aspects, the second cell type is keratinocytes and the 3-D culture is cultured until the keratinocytes form layer at least 150 μm in depth (as measured from the point the microfeatures extend most fully into the layer comprising the keratinocytes.

In some embodiments of any of the aspects, the second cell type is keratinocytes and the 3-D culture is cultured for at least 1 week. In some embodiments of any of the aspects, the second cell type is keratinocytes and the 3-D culture is cultured for at least 2 weeks. In some embodiments of any of the aspects, the second cell type is keratinocytes and the 3-D culture is cultured for at least 3 weeks.

In some embodiments of any of the aspects, the first cell type is fibroblasts and the second cell type is keratinocytes and the culturing of the 3-D culture comprises a first phase of culturing the entire 3-D culture submerged in media and second phase of culturing the 3-D culture such that at least one surface of the keratinocytes (e.g., the top face of the surface of the keratinocytes) are at an air-liquid interface. This can be accomplished by culture in a Transwell insert that is raised in the media to begin the second phase and/or by decreasing the level of the media in the culture container.

Prior attempts to create ex vivo cultures with microfeatures have provided poor results or disadvantageous methods which the present methods can avoid, thereby providing cultures which more accurately mimic 3-D cultures featuring microfeatures.

For example, in some embodiments of any of the aspects, the methods described herein do not comprise exposing the gel or pre-gel to a laser and/or does not comprise laser ablating the gel or pre-gel. Such laser ablation can damage the cells in the gel and cannot achieve the resolution possible with the present methods. Laser ablation also requires the use of certain cross-linked collagens, while the present methods are not limited to these specific gels.

In some embodiments of any of the aspects, the methods described herein do not comprise cross-linking components (e.g., collagen of the gel or pre-gel). In some embodiments of any of the aspects, the cross-linking comprises dehydration and/or thermal treatment. In some embodiments of any of the aspects, the cross-linking comprises the use of a carbodiimde, e.g., 1-ethyl-3-(3-dimethylaminopropyl)carbodiimide hydrochloride (EDC). In some embodiments of any of the aspects, the gel or pre-gel does not comprise PEG-diacrylate and/or is not exposed to ultraviolet light treatment or near-UV light treatment (e.g., near-UV blue light such as 405 nm).

In some embodiments of any of the aspects, the pre-gel comprises telocollagen and the pre-gel or gel is crosslinked. In some embodiments of any of the aspects, the pre-gel comprises telocollagen and fibrinogen and the pre-gel or gel is crosslinked. In some embodiments of any of the aspects, the pre-gel comprises telocollagen and the pre-gel or gel is crosslinked with carbodiimde, e.g., 1-ethyl-3-(3-dimethylaminopropyl)carbodiimide hydrochloride (EDC). In some embodiments of any of the aspects, the pre-gel comprises telocollagen and fibrinogen and the pre-gel or gel is crosslinked with carbodiimde, e.g., 1-ethyl-3-(3-dimethylaminopropyl)carbodiimide hydrochloride (EDC).

In some embodiments of any of the aspects, the pre-gel does not comprise atelocollagen and the pre-gel or gel is crosslinked. In some embodiments of any of the aspects, the pre-gel comprises telocollagen and does not comprise atelocollagen, and the pre-gel or gel is crosslinked. In some embodiments of any of the aspects, the pre-gel comprises telocollagen and fibrinogen, and does not comprise atelocollagen, and the pre-gel or gel is crosslinked.

In some embodiments of any of the aspects, the pre-gel does not comprise atelocollagen and the pre-gel or gel is crosslinked with carbodiimde, e.g., 1-ethyl-3-(3-dimethylaminopropyl)carbodiimide hydrochloride (EDC). In some embodiments of any of the aspects, the pre-gel comprises telocollagen and does not comprise atelocollagen, and the pre-gel or gel is crosslinked with carbodiimde, e.g., 1-ethyl-3-(3-dimethylaminopropyl)carbodiimide hydrochloride (EDC). In some embodiments of any of the aspects, the pre-gel comprises telocollagen and fibrinogen, and does not comprise atelocollagen, and the pre-gel or gel is crosslinked with carbodiimde, e.g., 1-ethyl-3-(3-dimethylaminopropyl)carbodiimide hydrochloride (EDC).

In some embodiments of any of the aspects, the at least a second type of cell or the culture does not comprise HaCaT keratinocytes, which cannot differentiate into full epidermis.

In some embodiments of any of the aspects, the methods described herein do not comprise a step of grafting into or onto an organism.

The methods and compositions described herein and be used to examine or measure the response of cultures to one or more stimuli. For example, in 3-D cultures comprising a first layer of fibroblasts and a second layer of keratinocytes and/or differentiated keratinocytes, the responses of dermis/epidermis tissues can be modeled, e.g., to study wound healing or whether a compound is a skin irritant. In one aspect of any of the embodiments, described herein is a method comprising: a) preparing a multi-layer (e.g, 2 layer) 3-D culture according to the methods described herein; applying a stimulus comprising a candidate agent, mechanical stress, or trauma to the multi-layer 3-D culture; and optionally, measuring or observing one or more responses of the multi-layer 3-D culture to the stimulus.

The measuring or observing one or more responses can comprise visual observation by eye or by microscopy, or measurement of visually observed features, or measurement of the levels or activity of biomolecules. The measuring or observing one or more response can also comprise measuring or observing a detectable signal or label expressed by or comprised by one or more of the cell types, e.g, is the result of a reporter gene. Merely by way of example, keratinocytes expressing GFP can be used to track keratinocyte migration and proliferation, e.g., during a wound healing process. As a further illustrative example, keratinocytes expressing GFP under the control of the TGF-beta promoter or as a fusion protein with TGF-beta can be used to track keratinocyte migration and proliferation, e.g., during a wound healing process.

As used herein, the terms “candidate compound” or “candidate agent” refer to a compound or agent and/or compositions thereof that are to be screened and/or analyzed for their affect on the 3-D culture. As used herein, the terms “compound” or “agent” are used interchangeably and refer to molecules and/or compositions. The compounds/agents include, but are not limited to, chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or animal cells or tissues; naturally occurring or synthetic compositions; peptides; aptamers; and antibodies and intrabodies, or fragments thereof.

Generally, compounds can be tested at any concentration. In some embodiments, compounds are tested at concentration in the range of about 0.1 nM to about 1000 mM. In one embodiment, the compound is tested in the range of about 0.1 μM to about 20 μM, about 0.1 μM to about 10 μM, or about 0.1 μM to about 5 μM. In one embodiment, compounds are tested at 1 μM. Candidate agents can be introduce free in solution in the media, or in pharmaceutically relevant formulations, e.g., liposomes, nanoparticles, controlled-release formulations, etc. For the methods described herein, test compounds may be screened individually, or in groups. Group screening is particularly useful where hit rates for effective test compounds are expected to be low such that one would not expect more than one positive result for a given group.

Candidate agents can be produced recombinantly using methods well known to those of skill in the art (see Sambrook et al., Molecular Cloning: A Laboratory Manual (2 ed.), Cold Spring Harbor Laboratory Press, Cold Spring Harbor, N.Y., USA (1989)). Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule (Woburn, MA), Panvera (Madison, WI), Ryan Scientific (Mt. Pleasant, SC), and Enzo Life Sciences (Plymouth Meeting, PA).

Another aspect of the technology described herein relates to kits for performing the methods described herein and/or preparing the 3-D cultures described herein. Described herein are kit components that can be included in one or more of the kits described herein. In some embodiments, the components described herein can be provided singularly or in any combination as a kit.

In some embodiments of any of the aspects, the kit comprises one or more stamps comprising at least one face comprising one or more microfeatures. In some embodiments of any of the aspects, the kit comprises at least one cell culture container comprising at least one cell growth area and the stamp is sized to be inserted into the cell growth area.

The kit of any of the preceding claims, further comprising one or more of: a pre-gel, media, a lift spacer (e.g., for lifting a Transwell insert or the like to a new height in a cell culture well), a first type of cell, and a second type of cell.

In some embodiments, the kit comprises an drug as described herein (e.g., caffeine, abscisic acid, rapamycin, gibberellin, protease inhibitor, or analogs thereof). In some

In some embodiments, the compositions in the kit can be provided in a watertight or gas tight container which in some components are substantially free of other components of the kit. For example, a composition can be supplied in more than one container, e.g., it can be supplied in a container having sufficient reagent for a predetermined number of cell culture events, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

In addition, the kit optionally comprises informational material. The informational material can be descriptive, instructional, marketing or other material that relates to the methods described herein. The informational material of the kits is not limited in its form. In one embodiment, the informational material can include information about production of the components, concentration, date of expiration, batch or production site information, and so forth. In one embodiment, the informational material relates to methods for using or administering the components of the kit.

The kit can be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments of any of the aspects, a cell “obtained from” a specified organism refers to a cell isolated by physical or chemical means from that organism, and the progeny of that originally isolated cell which retain the characteristics of that cell. In some embodiments of any of the aspects, a cell “derived from” a specified organism is descended from a cell obtained from the specified organism but which as undergone changes ex vivo, e.g, genetic engineering, differentiation, or dedifferentiation. Accordingly, the cell “derived” from a specified organism can be identified as having key genetic or phenotype characertistics of the source species but many have alterations or additions not found in the source species naturally.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects. A subject can be male or female.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

“Marker” in the context of the present invention refers to an expression product, e.g., nucleic acid or polypeptide which is differentially present in a cell, tissue, or organism in a certain states as compared to a comparable sample taken from control subjects in a different state. The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, a epidermal skin sampling technique, and/or collection of a secreted or expelled bodily fluid (e.g., mucus, urine, sweat, etc), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:

1. A method comprising:
a) applying at least one face of a stamp onto at least one surface of a pre-gel comprising at least a first type of cell, wherein the at least one face of the stamp comprises one or more microfeatures;
b) maintaining contact of the at least one face of the stamp with the at least one surface of the pre-gel while the pre-gel forms a gel;
c) separating the at least one face of the stamp and the at least one surface of the gel formed in step b) and thereby providing a gel comprising at least one stamped surface; and
d) contacting the at least one stamped surface of the gel resulting from step c) with at least a second type of cell to provide a two layer 3-D culture.

2. The method of paragraph 1, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

3. The method of any of the preceding paragraphs, wherein the first type of cell is fibroblasts and the second type of cell is keratinocytes.

4. The method of paragraph 3, further comprising a step e) of culturing the 3-D culture until the keratinocytes form a stratified epidermis layer.

5. The method of paragraph 3, further comprising a step e) of culturing the 3-D culture until a basement membrane has formed.

6. The method of paragraph 4 or 5, wherein step e) comprises culturing the 3-D culture for at least 2 weeks.

7. The method of paragraph 4 or 5, wherein step e) comprises a first phase of culturing the entire 3-D culture submerged in media and second phase of culturing the 3-D culture such that at least one surface of the keratinocytes are at an air-liquid interface.

8. The method of any of paragraphs 4-7, wherein step e) comprises culturing the 3-D culture until the keratinocytes form a layer that is at least 100 μm in depth.

9. The method of any of paragraphs 4-7, wherein step e) comprises culturing the 3-D culture until the keratinocytes form a layer that is at least 150 μm in depth.

10. The method of any of paragraphs 1-9, further comprising a step e) of culturing the 3-D culture until the second type of cell forms a layer that is at least 50 μm in depth.

11. The method of paragraph 10, wherein step e) comprises culturing the 3-D culture until the layer is at least 100 μm in depth.

12. The method of any of the preceding paragraphs, wherein the pre-gel comprises one or more of:
collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing.

13. The method of paragraph 12, wherein the pre-gel comprises one of the formulations of Table 2 or Table 3.

14. The method of paragraph 12, wherein the gel or pre-gel comprises more than 6 mg/mL of collagen, atellocollagen, or telocollagen.

15. The method of paragraph 12, wherein the gel or pre-gel comprises more than 8 mg/mL of collagen, atellocollagen, or telocollagen.

16. The method of any of the preceding paragraphs, wherein the gel or pre-gel has a depth of from about 100 μm to 10 mm.

17. The method of any of the preceding paragraphs, wherein the gel or pre-gel has a depth of from about 500 μm to 4 mm.

18. The method of any of the preceding paragraphs, wherein the gel or pre-gel has a depth of from about 500 μm to 2 mm.

19. The method of any of the preceding paragraphs, wherein the pre-gel is provided in a Transwell insert.

20. The method of any of the preceding paragraphs, wherein each microfeature extends 1 μm to 1 mm from the face of the stamp.

21. The method of any of the preceding paragraphs, wherein each microfeature extends 100 μm to 400 μm from the face of the stamp.

22. The method of any of the preceding paragraphs, wherein each microfeature has a width and/or length of at least 100 μm.

23. The method of any of the preceding paragraphs, wherein each microfeature has a height which is no more than 5× the narrowest dimension of the microfeature.

24. The method of any of the preceding paragraphs, wherein each microfeature forms a shape on the face of the stamp that is a polyhedron, a hexahedron, a cube, a ridge, a wall, a shape created by introducing a curve or angle into any of the preceding shapes, or a shape created by rounding one or more of the edges of one of the preceding shapes.

25. The method of any of the preceding paragraphs, wherein the method does not comprise exposing the gel or pre-gel to a laser and/or does not comprise laser ablating the gel or pre-gel.

26. The method of any of the preceding paragraphs, wherein the pre-gel does not comprise and the culture is not contacted with HaCaT keratinocytes.

27. The method of any of the preceding paragraphs, wherein the gel or pre-gel does not comprise PEG-diacrylate and/or is not exposed to ultraviolet light or near-UV light treatment.

28. The method of any of the preceding paragraphs, wherein the method does not comprise a step of grafting into or onto an organism.

29. The method of any of the preceding paragraphs, wherein the method does not comprise cross-linking components of the gel or pre-gel.

30. The method of paragraph 29, wherein the cross-linking is the cross-linking of collagen.

31. The method of paragraph 29 or 30, wherein the cross-linking comprises dehydration and/or thermal treatment.

32. The method of any of paragraphs 29-31, wherein the cross-linking comprises the use of a carbodiimde, e.g., 1-ethyl-3-(3-dimethylaminopropyl)carbodiimide hydrochloride (EDC).

33. A method comprising:
a) preparing a two-layer 3-D culture according to any of the preceding paragraphs;
b) applying a stimulus comprising a candidate agent, mechanical stress, or trauma to the two-layer 3-D culture;
c) and optionally, measuring or observing one or more responses of the two-layer 3-D culture to the stimulus.

34. The method of paragraph 33, wherein one or more of the cell types comprises or expresses a detectable label and the measuring or observing comprises detecting the label.

35. A two layer 3-D culture comprising:
a) a first layer comprising at least a first type of cell in a gel;
b) a second layer comprising at least a second type of cell;
c) a boundary between the first and second layers comprising one or more microfeatures.

36. The culture of paragraph 35, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

37. The culture of any of the preceding paragraphs, wherein the at least a first type of cell comprises fibroblasts and the at least a second type of cell comprises keratinocytes.

38. The culture of paragraph 37, wherein the keratinocytes form a stratified epidermis layer.

39. The culture of paragraph 37, wherein a basement membrane is present.

40. The culture of any of paragraphs 35-39, wherein the second layer is at least 100 μm in depth.

41. The culture of any of paragraphs 35-40, wherein the second layer is at least 150 μm in depth.

42. The culture of any of paragraphs 35-39, wherein the second layer is at least 50 μm in depth.

43. The culture of any of the preceding paragraphs, wherein the first layer comprises one or more of:
collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing.

44. The culture of paragraph 43, wherein the first layer comprises one of the formulations of Table 2 or Table 3.

45. The culture of any one of paragraphs 43-44, wherein the first layer comprises more than 6 mg/mL of collagen, atellocollagen, or telocollagen.

46. The culture of any one of paragraphs 43-44, wherein the first layer comprises more than 8 mg/mL of collagen, atellocollagen, or telocollagen.

47. The culture of any one of paragraphs 43-46, wherein the first layer has a depth of from about 100 μm to 10 mm.

48. The culture of any one of paragraphs 43-46, wherein the first layer has a depth of from about 500 μm to 4 mm.

49. The culture of any one of paragraphs 43-46, wherein the first layer has a depth of from about 500 μm to 2 mm.

50. The culture of any one of paragraphs 43-44, wherein the culture is in a Transwell insert.

51. The culture of any of the preceding paragraphs, wherein each microfeature extends 1 μm to 1 mm into one of the layers.

52. The culture of any of the preceding paragraphs, wherein each microfeature extends 100 μm to 400 μm into one of the layers.

53. The culture of any of the preceding paragraphs, wherein each microfeature has a width and/or length of at least 100 μm.

54. The culture of any of the preceding paragraphs, wherein each microfeature has a height which is no more than 5× the narrowest dimension of the microfeature.

55. The culture of any of the preceding paragraphs, wherein each microfeature forms a shape that is a polyhedron, a hexahedron, a cube, a ridge, a wall, a shape created by introducing a curve or angle into any of the preceding shapes, or a shape created by rounding one or more of the edges of one of the preceding shapes.

56. The culture of any of the preceding paragraphs, wherein the culture does not comprise HaCaT keratinocytes.

57. The culture of any of the preceding paragraphs, wherein the culture does not comprise PEG-diacrylate.

58. The culture of any of the preceding paragraphs, wherein the culture does not comprise cross-linked components.

59. A kit comprising one or more stamps comprising at least one face comprising one or more microfeatures.

60. The kit of any of the preceding paragraphs, wherein the kit further comprises at least one cell culture container comprising at least one cell growth area and the stamp is sized to be inserted into the cell growth area.

61. The kit of any of the preceding paragraphs, further comprising one or more of: a pre-gel, media, a lift spacer, a first type of cell, and a second type of cell.

In some embodiments, the present technology may be defined in any of the following numbered paragraphs:

1. A method comprising:
a) applying at least one face of a stamp onto at least one surface of a pre-gel comprising at least a first type of cell, wherein the at least one face of the stamp comprises one or more microfeatures;
b) maintaining contact of the at least one face of the stamp with the at least one surface of the pre-gel while the pre-gel forms a gel;
c) separating the at least one face of the stamp and the at least one surface of the gel formed in step b) and thereby providing a gel comprising at least one stamped surface; and
d) contacting the at least one stamped surface of the gel resulting from step c) with at least a second type of cell to provide a two layer 3-D culture.

2. The method of paragraph 1, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

3. The method of any of the preceding paragraphs, wherein the first type of cell is fibroblasts and the second type of cell is keratinocytes.

4. The method of paragraph 3, further comprising a step e) of culturing the 3-D culture until the keratinocytes form a stratified epidermis layer.

5. The method of paragraph 3, further comprising a step e) of culturing the 3-D culture until a basement membrane has formed.

6. The method of paragraph 4 or 5, wherein step e) comprises culturing the 3-D culture for at least 2 weeks.

7. The method of paragraph 4 or 5, wherein step e) comprises a first phase of culturing the entire 3-D culture submerged in media and second phase of culturing the 3-D culture such that at least one surface of the keratinocytes are at an air-liquid interface.

8. The method of any of paragraphs 4-7, wherein step e) comprises culturing the 3-D culture until the keratinocytes form a layer that is at least 100 μm in depth.

9. The method of any of paragraphs 4-7, wherein step e) comprises culturing the 3-D culture until the keratinocytes form a layer that is at least 150 μm in depth.

10. The method of any of paragraphs 1-9, further comprising a step e) of culturing the 3-D culture until the second type of cell forms a layer that is at least 50 μm in depth.

11. The method of paragraph 10, wherein step e) comprises culturing the 3-D culture until the layer is at least 100 μm in depth.

12. The method of any of the preceding paragraphs, wherein the pre-gel comprises one or more of:
collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing.

13. The method of paragraph 12, wherein the pre-gel comprises one of the formulations of Table 2 or Table 3.

14. The method of paragraph 12, wherein the gel or pre-gel comprises more than 6 mg/mL of collagen, atellocollagen, or telocollagen.

15. The method of paragraph 12, wherein the gel or pre-gel comprises more than 8 mg/mL of collagen, atellocollagen, or telocollagen.

16. The method of any of the preceding paragraphs, wherein the gel or pre-gel has a depth of from about 100 μm to 10 mm.

17. The method of any of the preceding paragraphs, wherein the gel or pre-gel has a depth of from about 500 μm to 4 mm.

18. The method of any of the preceding paragraphs, wherein the gel or pre-gel has a depth of from about 500 μm to 2 mm.

19. The method of any of the preceding paragraphs, wherein the pre-gel is provided in a Transwell insert.

20. The method of any of the preceding paragraphs, wherein each microfeature extends 1 μm to 1 mm from the face of the stamp.

21. The method of any of the preceding paragraphs, wherein each microfeature extends 100 μm to 400 μm from the face of the stamp.

22. The method of any of the preceding paragraphs, wherein each microfeature has a width and/or length of at least 100 μm.

23. The method of any of the preceding paragraphs, wherein each microfeature has a height which is no more than 5× the narrowest dimension of the microfeature.

24. The method of any of the preceding paragraphs, wherein each microfeature forms a shape on the face of the stamp that is a polyhedron, a hexahedron, a cube, a ridge, a wall, a shape created by introducing a curve or angle into any of the preceding shapes, or a shape created by rounding one or more of the edges of one of the preceding shapes.

25. The method of any of the preceding paragraphs, wherein the method does not comprise exposing the gel or pre-gel to a laser and/or does not comprise laser ablating the gel or pre-gel.

26. The method of any of the preceding paragraphs, wherein the pre-gel does not comprise and the culture is not contacted with HaCaT keratinocytes.

27. The method of any of the preceding paragraphs, wherein the gel or pre-gel does not comprise PEG-diacrylate and/or is not exposed to ultraviolet light or near-UV light treatment.

28. The method of any of the preceding paragraphs, wherein the method does not comprise a step of grafting into or onto an organism.

29. The method of any of the preceding paragraphs, wherein the method does not comprise cross-linking components of the gel or pre-gel.

30. The method of paragraph 29, wherein the cross-linking is the cross-linking of collagen.

31. The method of paragraph 29 or 30, wherein the cross-linking comprises dehydration and/or thermal treatment.

32. The method of any of paragraphs 29-31, wherein the cross-linking comprises the use of a carbodiimde, e.g., 1-ethyl-3-(3-dimethylaminopropyl)carbodiimide hydrochloride (EDC).

33. A method comprising:
a) preparing a two-layer 3-D culture according to any of the preceding paragraphs;
b) applying a stimulus comprising a candidate agent, mechanical stress, or trauma to the two-layer 3-D culture;
c) and optionally, measuring or observing one or more responses of the two-layer 3-D culture to the stimulus.

34. The method of paragraph 33, wherein one or more of the cell types comprises or expresses a detectable label and the measuring or observing comprises detecting the label.

35. A two layer 3-D culture comprising:
a) a first layer comprising at least a first type of cell in a gel;
b) a second layer comprising at least a second type of cell;
c) a boundary between the first and second layers comprising one or more microfeatures.

36. The culture of paragraph 35, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

37. The culture of any of the preceding paragraphs, wherein the at least a first type of cell comprises fibroblasts and the at least a second type of cell comprises keratinocytes.

38. The culture of paragraph 37, wherein the keratinocytes form a stratified epidermis layer.

39. The culture of paragraph 37, wherein a basement membrane is present.

40. The culture of any of paragraphs 35-39, wherein the second layer is at least 100 μm in depth.

41. The culture of any of paragraphs 35-40, wherein the second layer is at least 150 μm in depth.

42. The culture of any of paragraphs 35-39, wherein the second layer is at least 50 μm in depth.

43. The culture of any of the preceding paragraphs, wherein the first layer comprises one or more of:
collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing.

44. The culture of paragraph 43, wherein the first layer comprises one of the formulations of Table 2 or Table 3.

45. The culture of any one of paragraphs 43-44, wherein the first layer comprises more than 6 mg/mL of collagen, atellocollagen, or telocollagen.

46. The culture of any one of paragraphs 43-44, wherein the first layer comprises more than 8 mg/mL of collagen, atellocollagen, or telocollagen.

47. The culture of any one of paragraphs 43-46, wherein the first layer has a depth of from about 100 μm to 10 mm.

48. The culture of any one of paragraphs 43-46, wherein the first layer has a depth of from about 500 μm to 4 mm.

49. The culture of any one of paragraphs 43-46, wherein the first layer has a depth of from about 500 μm to 2 mm.

50. The culture of any one of paragraphs 43-44, wherein the culture is in a Transwell insert.

51. The culture of any of the preceding paragraphs, wherein each microfeature extends 1 μm to 1 mm into one of the layers.

52. The culture of any of the preceding paragraphs, wherein each microfeature extends 100 μm to 400 μm into one of the layers.

53. The culture of any of the preceding paragraphs, wherein each microfeature has a width and/or length of at least 100 μm.

54. The culture of any of the preceding paragraphs, wherein each microfeature has a height which is no more than 5× the narrowest dimension of the microfeature.

55. The culture of any of the preceding paragraphs, wherein each microfeature forms a shape that is a polyhedron, a hexahedron, a cube, a ridge, a wall, a shape created by introducing a curve or angle into any of the preceding shapes, or a shape created by rounding one or more of the edges of one of the preceding shapes.

56. The culture of any of the preceding paragraphs, wherein the culture does not comprise HaCaT keratinocytes.

57. The culture of any of the preceding paragraphs, wherein the culture does not comprise PEG-diacrylate.

58. The culture of any of the preceding paragraphs, wherein the culture does not comprise cross-linked components.

59. A two layer 3-D culture comprising:
a) a first layer comprising at least a first type of cell in a gel comprising telocollagen, fibrinogen, NaOH, and thrombin;
b) a second layer comprising at least a second type of cell.

60. The culture of paragraph 59, wherein the gel comprising telocollagen, fibrinogen, and thrombin comprises:
a) telocollagen at 3-20 mg/mL
b) fibrinogen at 2.5-20 mg/mL;
c) NaOH at 0.005-0.1 mol/L;
d) PBS at 0.5-2×; and
e) thrombin at 0.125-1 U/mL.

61. The culture of paragraph 59, wherein the gel comprising telocollagen, fibrinogen, and thrombin comprises:
a) telocollagen at 6-10 mg/mL
b) fibrinogen at 5-10 mg/mL;
c) NaOH at 0.01-0.05 mol/L;
d) PBS at 1×; and
e) thrombin at 0.25-0.5 U/mL.

62. The culture of any one of paragraphs 59-61, wherein the first cells are present in the gel at a concentration of 0-8 million cells/mL.

63. The culture of any one of paragraphs 59-61, wherein the first cells are present in the gel at a concentration of 1-4 million cells/mL.

64. The culture of any one of paragraphs 59-61, wherein the first cells are present in the gel at a concentration of 0-4 million cells/mL.

65. The culture of any one of paragraphs 59-64, wherein the second cells are a confluent layer.

66. The culture of any one of paragraphs 59-64, wherein the second cells are a cellular monolayer.

67. The culture of any one of paragraphs 59-64, wherein the second cells are a cellular multilayer.

68. The culture of any one of paragraphs 59-64, wherein the second cells are a multi-layered epidermis.

69. The culture of any one of paragraphs 59-64, wherein the second cells form a 10 micron to 150 micron layer.

70. The culture of any one of paragraphs 59-69, wherein the second layer does not comprise a gel.

71. The culture of any of paragraphs 59-70, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

72. The culture of any of paragraphs 59-71, wherein the at least a first type of cell comprises fibroblasts and the at least a second type of cell comprises keratinocytes.

73. The culture of paragraph 72, wherein the keratinocytes form a stratified epidermis layer.

74. The culture of paragraph 72 or 73, wherein a basement membrane is present.

75. The culture of any of paragraphs 59-74, wherein the second layer is at least 100 μm in depth.

76. The culture of any of paragraphs 59-74, wherein the second layer is at least 150 μm in depth.

77. The culture of any of paragraphs 59-74, wherein the second layer is at least 50 μm in depth.

78. The culture of any one of paragraphs 59-77, wherein the first layer has a depth of from about 100 μm to 10 mm.

79. The culture of any one of paragraphs 59-77, wherein the first layer has a depth of from about 500 μm to 4 mm.

80. The culture of any one of paragraphs 59-77, wherein the first layer has a depth of from about 500 μm to 2 mm.

81. The culture of any one of paragraphs 59-80, wherein the culture is in a Transwell insert.

82. The culture of any one of paragraphs 59-81, wherein the culture does not comprise HaCaT keratinocytes.

83. The culture of any one of paragraphs 59-82, wherein the culture does not comprise PEG-diacrylate.

84. The culture of any one of paragraphs 59-83, wherein the culture does not comprise cross-linked components.

85. A kit comprising one or more stamps comprising at least one face comprising one or more microfeatures.

86. The kit of any of the preceding paragraphs, wherein the kit further comprises at least one cell culture container comprising at least one cell growth area and the stamp is sized to be inserted into the cell growth area.

87. The kit of any of the preceding paragraphs, further comprising one or more of: a pre-gel, media, a lift spacer, a first type of cell, and a second type of cell.

In some embodiments, the present technology may be defined in any of the following numbered paragraphs:

1. A method comprising:
a) applying at least one face of a stamp onto at least one surface of a pre-gel comprising at least a first type of cell, wherein the at least one face of the stamp comprises one or more microfeatures;
b) maintaining contact of the at least one face of the stamp with the at least one surface of the pre-gel while the pre-gel forms a gel;
c) separating the at least one face of the stamp and the at least one surface of the gel formed in step b) and thereby providing a gel comprising at least one stamped surface; and
d) contacting the at least one stamped surface of the gel resulting from step c) with at least a second type of cell to provide a two layer 3-D culture.

2. The method of paragraph 1, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

3. The method of any one of the preceding paragraphs, wherein the first type of cell is fibroblasts and the second type of cell is keratinocytes.

4. The method of paragraph 3, further comprising a step e) of culturing the 3-D culture until the keratinocytes form a stratified epidermis layer.

5. The method of paragraph 3, further comprising a step e) of culturing the 3-D culture until a basement membrane has formed.

6. The method of paragraph 4 or 5, wherein step e) comprises culturing the 3-D culture for at least 2 weeks.

7. The method of paragraph 4 or 5, wherein step e) comprises a first phase of culturing the entire 3-D culture submerged in media and second phase of culturing the 3-D culture such that at least one surface of the keratinocytes are at an air-liquid interface.

8. The method of any one of paragraphs 4-7, wherein step e) comprises culturing the 3-D culture until the keratinocytes form a layer that is at least 100 μm in depth.

9. The method of any one of paragraphs 4-7, wherein step e) comprises culturing the 3-D culture until the keratinocytes form a layer that is at least 150 μm in depth.

10. The method of any one of paragraphs 1-9, further comprising a step e) of culturing the 3-D culture until the second type of cell forms a layer that is at least 50 μm in depth.

11. The method of paragraph 10, wherein step e) comprises culturing the 3-D culture until the layer is at least 100 μm in depth.

12. The method of any one of the preceding paragraphs, wherein the pre-gel comprises one or more of:
collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing.

13. The method of any one of the preceding paragraphs, wherein the pre-gel comprises telocollagen and fibrin.

14. The method of any one of the preceding paragraphs, wherein the pre-gel does not comprise atelocollagen.

15. The method of any one of paragraphs 12-14, wherein the pre-gel comprises one of the formulations of Table 2 or Table 3.

16. The method of any one of paragraphs 12-15, wherein the gel or pre-gel comprises more than 6 mg/mL of collagen, atellocollagen, or telocollagen.

17. The method of any one of paragraphs 12-16, wherein the gel or pre-gel comprises more than 8 mg/mL of collagen, atellocollagen, or telocollagen.

18. The method of any one of the preceding paragraphs, wherein the gel or pre-gel has a depth of from about 100 μm to 10 mm.

19. The method of any one of the preceding paragraphs, wherein the gel or pre-gel has a depth of from about 500 μm to 4 mm.

20. The method of any one of the preceding paragraphs, wherein the gel or pre-gel has a depth of from about 500 μm to 2 mm.

21. The method of any one of the preceding paragraphs, wherein the pre-gel is provided in a Transwell insert.

22. The method of any one of the preceding paragraphs, wherein each microfeature extends 1p m to 1 mm from the face of the stamp.

23. The method of any one of the preceding paragraphs, wherein each microfeature extends 100 μm to 400 μm from the face of the stamp.

24. The method of any one of the preceding paragraphs, wherein each microfeature extends at least 300 μm from the face of the stamp.

25. The method of any one of the preceding paragraphs, wherein each microfeature extends 300-500 μm from the face of the stamp.

26. The method of any one of the preceding paragraphs, wherein each microfeature extends at least 400 μm from the face of the stamp.

27. The method of any one of the preceding paragraphs, wherein each microfeature extends 400-500 μm from the face of the stamp.

28. The method of any one of the preceding paragraphs, wherein each microfeature has a width and/or length of at least 100 μm.

29. The method of any one of the preceding paragraphs, wherein each microfeature has a width and/or length of at least 400 μm.

30. The method of any one of the preceding paragraphs, wherein each microfeature has a width and/or length 400 to 500 μm.

31. The method of any one of the preceding paragraphs, wherein each microfeature has a height which is no more than 5× the narrowest dimension of the microfeature.

32. The method of any one of the preceding paragraphs, wherein each microfeature forms a shape on the face of the stamp that is a polyhedron, a hexahedron, a cube, a ridge, a wall, a shape created by introducing a curve or angle into any of the preceding shapes, or a shape created by rounding one or more of the edges of one of the preceding shapes.

33. The method of any one of the preceding paragraphs, wherein the method does not comprise exposing the gel or pre-gel to a laser and/or does not comprise laser ablating the gel or pre-gel.

34. The method of any one of the preceding paragraphs, wherein the pre-gel does not comprise and the culture is not contacted with HaCaT keratinocytes.

35. The method of any one of the preceding paragraphs, wherein the gel or pre-gel does not comprise PEG-diacrylate and/or is not exposed to ultraviolet light or near-UV light treatment.

36. The method of any one of the preceding paragraphs, wherein the method does not comprise a step of grafting into or onto an organism.

37. The method of any one of the preceding paragraphs, wherein the method does not comprise cross-linking components of the gel or pre-gel.

38. The method of any one of the preceding paragraphs, wherein the method further comprises cross-linking components of the gel or pre-gel.

39. The method of paragraph 37 or 38, wherein the cross-linking is the cross-linking of collagen.

40. The method of any one of paragraphs 37-39, wherein the cross-linking comprises dehydration and/or thermal treatment.

41. The method of any one of paragraphs 37-40, wherein the cross-linking comprises the use of a carbodiimde, e.g., 1-ethyl-3-(3-dimethylaminopropyl)carbodiimide hydrochloride (EDC).

42. A method comprising:
a) preparing a two-layer 3-D culture according to any of the preceding paragraphs;
b) applying a stimulus comprising a candidate agent, mechanical stress, or trauma to the two-layer 3-D culture;
c) and optionally, measuring or observing one or more responses of the two-layer 3-D culture to the stimulus.

43. The method of paragraph 42, wherein one or more of the cell types comprises or expresses a detectable label and the measuring or observing comprises detecting the label.

44. A two layer 3-D culture comprising:
a) a first layer comprising at least a first type of cell in a gel;
b) a second layer comprising at least a second type of cell;
c) a boundary between the first and second layers comprising one or more microfeatures.

45. The culture of paragraph 44, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

46. The culture of any one of the preceding paragraphs, wherein the at least a first type of cell comprises fibroblasts and the at least a second type of cell comprises keratinocytes.

47. The culture of paragraph 46, wherein the keratinocytes form a stratified epidermis layer.

48. The culture of paragraph 46, wherein a basement membrane is present.

49. The culture of any one of paragraphs 44-48, wherein the second layer is at least 100 μm in depth.

50. The culture of any one of paragraphs 44-49, wherein the second layer is at least 150 μm in depth.

51. The culture of any one of paragraphs 44-50, wherein the second layer is at least 50 μm in depth.

52. The culture of any one of the preceding paragraphs, wherein the first layer comprises one or more of: collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing.

53. The culture of paragraph 52, wherein the first layer comprises one of the formulations of Table 2 or Table 3.

54. The culture of any one of paragraphs 52-53, wherein the first layer comprises telocollagen and fibrinogen.

55. The culture of any one of paragraphs 52-54, wherein the first layer does not comprise atelocollagen.

56. The culture of any one of paragraphs 52-55, wherein the first layer comprises more than 6 mg/mL of collagen, atellocollagen, or telocollagen.

57. The culture of any one of paragraphs 52-56, wherein the first layer comprises more than 8 mg/mL of collagen, atellocollagen, or telocollagen.

58. The culture of any one of paragraphs 52-57, wherein the first layer has a depth of from about 100 μm to 10 mm.

59. The culture of any one of paragraphs 52-58, wherein the first layer has a depth of from about 500 μm to 4 mm.

60. The culture of any one of paragraphs 52-58, wherein the first layer has a depth of from about 500 μm to 2 mm.

61. The culture of any one of paragraphs 52-60, wherein the culture is in a Transwell insert.

62. The culture of any one of the preceding paragraphs, wherein each microfeature extends 1p m to 1 mm into one of the layers.

63. The culture of any one of the preceding paragraphs, wherein each microfeature extends 100 μm to 400 μm into one of the layers.

64. The culture of any one of the preceding paragraphs, wherein each microfeature has a width and/or length of at least 100 μm.

65. The culture of any one of the preceding paragraphs, wherein each microfeature has a height which is no more than 5× the narrowest dimension of the microfeature.

66. The culture of any one of the preceding paragraphs, wherein each microfeature forms a shape that is a polyhedron, a hexahedron, a cube, a ridge, a wall, a shape created by introducing a curve or angle into any of the preceding shapes, or a shape created by rounding one or more of the edges of one of the preceding shapes.

67. The culture of any one of the preceding paragraphs, wherein the culture does not comprise HaCaT keratinocytes.

68. The culture of any one of the preceding paragraphs, wherein the culture does not comprise PEG-diacrylate.

69. The culture of any one of the preceding paragraphs, wherein the culture does not comprise cross-linked components.

70. The culture of any one of the preceding paragraphs, wherein the gel comprises cross-linked components.

71. The culture of paragraph 69 or 70, wherein the cross-linked components comprise cross-linked collagen.

72. The culture of any one of paragraphs 69-71, wherein the cross-linked components are cross-linked by dehydration and/or thermal treatment.

73. The culture of any one of paragraphs 69-71, wherein the cross-linked components are cross-linked by the use of a carbodiimde, e.g., 1-ethyl-3-(3-dimethylaminopropyl)carbodiimide hydrochloride (EDC).

74. A two layer 3-D culture comprising:
a) a first layer comprising at least a first type of cell in a gel comprising telocollagen, fibrinogen, NaOH, and thrombin;
b) a second layer comprising at least a second type of cell.

75. The culture of paragraph 74, wherein the gel comprising telocollagen, fibrinogen, and thrombin comprises:
a) telocollagen at 3-20 mg/mL
b) fibrinogen at 2.5-20 mg/mL;
c) NaOH at 0.005-0.1 mol/L;
d) PBS at 0.5-2×; and
e) thrombin at 0.125-1 U/mL.

76. The culture of paragraph 74, wherein the gel comprising telocollagen, fibrinogen, and thrombin comprises:
a) telocollagen at 6-10 mg/mL
b) fibrinogen at 5-10 mg/mL;
c) NaOH at 0.01-0.05 mol/L;
d) PBS at 1×; and
e) thrombin at 0.25-0.5 U/mL.

77. The culture of any one of paragraphs 74-76, wherein the first cells are present in the gel at a concentration of 0-8 million cells/mL.

78. The culture of any one of paragraphs 74-76, wherein the first cells are present in the gel at a concentration of 1-4 million cells/mL.

79. The culture of any one of paragraphs 74-76, wherein the first cells are present in the gel at a concentration of 0-4 million cells/mL.

80. The culture of any one of paragraphs 74-79, wherein the second cells are a confluent layer.

81. The culture of any one of paragraphs 74-79, wherein the second cells are a cellular monolayer.

82. The culture of any one of paragraphs 74-79, wherein the second cells are a cellular multilayer.

83. The culture of any one of paragraphs 74-79, wherein the second cells are a multi-layered epidermis.

84. The culture of any one of paragraphs 74-83, wherein the second cells form a 10 micron to 150 micron layer.

85. The culture of any one of paragraphs 74-84, wherein the second layer does not comprise a gel.

86. The culture of any one of paragraphs 74-85, wherein:
a) The at least a first type of cell is fibroblasts and the at least a second type of cell is keratinocytes;
b) The at least a first type of cell is fibroblasts and the at least a second type of cell is keratinocytes and melanocytes;
c) The at least a first type of cell is fibroblasts and the at least a second type of cell is melanocytes;
d) The at least a first type of cell is fibroblasts and endothelial cells and the at least a second type of cell is keratinocytes;
e) The at least a first type of cell is fibroblasts and endothelial cells and the at least a second type of cell is keratinocytes and melanocytes;
f) The at least a first type of cell is fibroblasts and endothelial cells and the at least a second type of cell is melanocytes;
g) The at least a first type of cell is fibroblasts and immune cells and the at least a second type of cell is keratinocytes;
h) The at least a first type of cell is fibroblasts and immune cells and the at least a second type of cell is keratinocytes and melanocytes;
i) The at least a first type of cell is fibroblasts and the at least a second type of cell is induced pluripotent stem cells;
j) The at least a first type of cell is induced pluripotent stem cells and the at least a second type of cell is induced pluripotent stem cells;
k) The at least a first type of cell is fibroblasts and the at least a second type of cell is cells differentiated from induced pluripotent stem cells;
l) The at least a first type of cell is cells differentiated from induced pluripotent stem cells and the at least a second type of cell is cells differentiated from induced pluripotent stem cells;
m) The at least a first type of cell is fibroblasts and the at least a second type of cell is heptatocytes;
n) The at least a first type of cell is fibroblasts and the at least a second type of cell is intestinal stem cells;
o) The at least a first type of cell is fibroblasts and the at least a second type of cell is intestinal epithelial cells;
p) The at least a first type of cell is fibroblasts and the at least a second type of cell is tumor cells
q) The at least a first type of cell is fibroblasts from a psoriasis patient and the at least a second type of cell is keratinocytes from a psoriasis patient; or
r) The at least a first type of cell is fibroblasts from a diabetic patient and the at least a second type of cell is keratinocytes from a diabetic patient.

87. The culture of any one of paragraphs 74-86, wherein the at least a first type of cell comprises fibroblasts and the at least a second type of cell comprises keratinocytes.

88. The culture of paragraph 87, wherein the keratinocytes form a stratified epidermis layer.

89. The culture of paragraph 87 or 88, wherein a basement membrane is present.

90. The culture of any one of paragraphs 74-89, wherein the second layer is at least 100 μm in depth.

91. The culture of any one of paragraphs 74-90, wherein the second layer is at least 150 μm in depth.

92. The culture of any one of paragraphs 74-90, wherein the second layer is at least 50 μm in depth.

93. The culture of any one of paragraphs 74-92, wherein the first layer has a depth of from about 100 μm to 10 mm.

94. The culture of any one of paragraphs 74-93, wherein the first layer has a depth of from about 500 μm to 4 mm.

95. The culture of any one of paragraphs 74-94, wherein the first layer has a depth of from about 500 μm to 2 mm.

96. The culture of any one of paragraphs 74-95, wherein the culture is in a Transwell insert.

97. The culture of any one of paragraphs 74-96, wherein the culture does not comprise HaCaT keratinocytes.

98. The culture of any one of paragraphs 74-97, wherein the culture does not comprise PEG-diacrylate.

99. The culture of any one of paragraphs 74-98, wherein the culture does not comprise cross-linked components.

100. The culture of any one of paragraphs 74-98, wherein the gel comprises cross-linked components.

101. The culture of paragraph 99 or 100, wherein the cross-linked components comprise cross-linked collagen.

102. The culture of any one of paragraphs 99-101, wherein the cross-linked components are cross-linked by dehydration and/or thermal treatment.

103. The culture of any one of paragraphs 99-101, wherein the cross-linked components are cross-linked by the use of a carbodiimde, e.g., 1-ethyl-3-(3-dimethylaminopropyl)carbodiimide hydrochloride (EDC).

104. A kit comprising one or more stamps comprising at least one face comprising one or more microfeatures.

105. The kit of any one of the preceding paragraphs, wherein the kit further comprises at least one cell culture container comprising at least one cell growth area and the stamp is sized to be inserted into the cell growth area.

106. The kit of any one of the preceding paragraphs, further comprising one or more of: a pre-gel, media, a lift spacer, a first type of cell, and a second type of cell.

The technology described herein is further illustrated by the following examples which in no way should be construed as being further limiting.

### EXAMPLES

### Example 1: Microstructured Skin Model with Rete Pegs

In vitro skin models have applications in pharmaceutical and cosmetics testing, disease modeling, and wound modeling. While significant progress has been made in the development of in vitro skin models, existing skin models typically have a flat interface between the two outer layers of skin, the dermis and epidermis. In human skin, the interface between the dermis and epidermis undulates, with protrusions called rete pegs extending from one layer into the other. Rete pegs are important to the mechanical properties of skin, as they improve the adhesion and increase the surface area between the two layers. Moreover, rete pegs are known to flatten with age, and skin disorders can change the structure of rete pegs. To model human skin, particularly the mechanical properties of skin, it is necessary to fabricate skin models with a variety of rete peg geometries. Described herein is a new method to fabricate skin models with flexibility in rete peg structure.

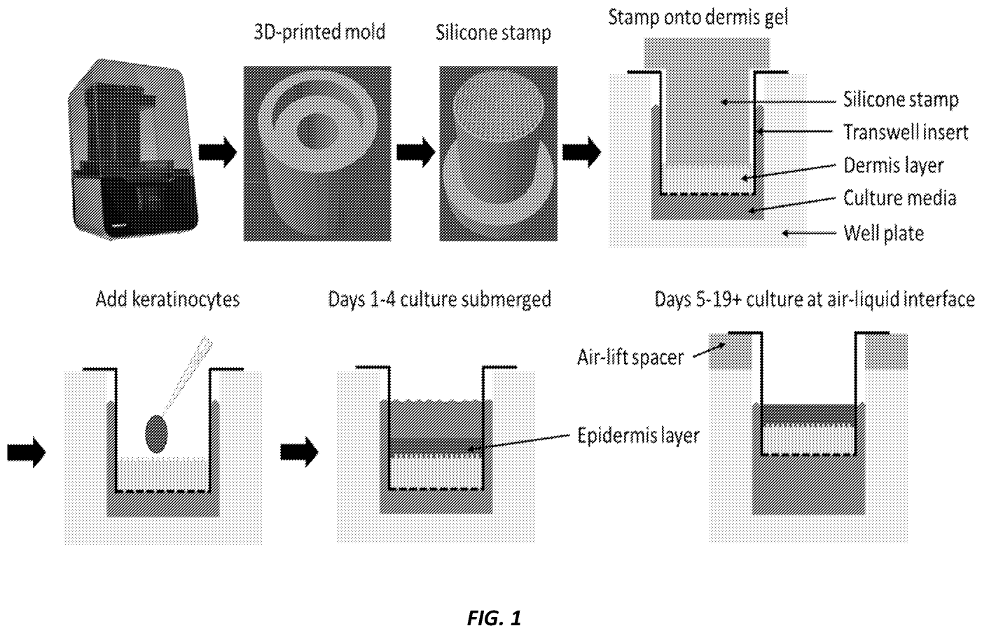

Described herein is a stamping procedure to imprint microfeatures (˜100-400 μm) into collagen, fibrin, and gelatin gels. The fabrication and culture process are shown in FIG. 1. First, a mold was 3D-printed with microfeatures using a commercial 3D printer. Second, the 3D-printed mold was used to generate a silicone stamp with the same microfeatures. Third, fibroblasts were encapsulated into a pre-gel and the stamp applied on top of this solution. After the sample gelled, the stamp was removed, leaving behind a gel with microwells of the same size as the stamp's microfeatures. This gel represents the dermis layer of skin. Fourth, to create the epidermis layer, keratinocytes were seeded on top of the dermis gel, which initially formed a monolayer. After culture for 2-3 weeks, the keratinocytes formed a stratified epidermis.

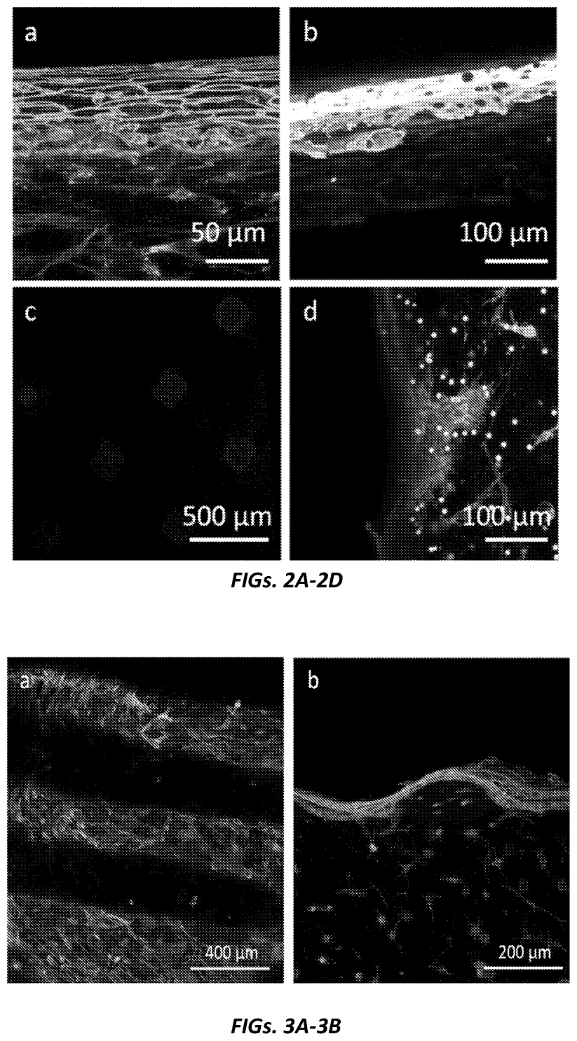

The keratinocytes form a multilayered and stratified epidermis on flat substrates (FIGS. 2A-2B). When cultured on substrates with peg-like geometries, square patterns are visible from a top-down view (FIG. 2C) and wells are visible from a cross section view (FIG. 2D).

Alternative geometries can be 3D printed to stamp onto the skin models. In FIG. 3, microchannels were stamped into the dermis layer and then keratinocytes added. From the top-down view (FIG. 3A) and the cross-sectional view (FIG. 3B), these features can be observed.

The critical difference between this model and existing models is the simple method for fabricating the rete peg structures. These structures make the model more human-like. It has been shown that epidermal stem cells respond to these undulating features 1, 2. This model can be used to draw a comparison between different patient groups, such as elderly patients, who have diminished rete pegs3, and young patients. It can also enhance applicability of a model to particular diseases, which might have abnormal rete peg structures. One example is psoriasis, which is characterized by elongated rete pegs4. It is contemplated herein that this model demonstrates better mechanical properties than a flat model because of the increased surface area between the dermis and epidermis. The model can be used in similar applications to existing skin models, such as skin irritation testing5, and proof-of-concept skin irritation testing has been performed.

The presently described methods and compositions are distinct from and have advantages over the prior art. For example, one work molded a channel geometry on a dense crosslinked collagen layer and seeded keratinocytes on top6. The channel geometry is unlike the usual peg-like geometry. Moreover, they molded this into a heavily crosslinked collagen material that was unable to culture living fibroblasts. Because of this, no basement membrane was produced between the dermis layer and epidermis layer. Another lab used laser micropatterning of a dense electrospun collagen layer to fabricate a rete peg geometry7, 8. The use of the laser was only able to fabricate two different geometries of rete pegs, and the laser method could damage the fibroblasts within the matrix. In addition, the laser used is not as accessible to most labs compared to a 3D printer.

### REFERENCES

1. Viswanathan, P., Guvendiren, M., Chua, W., Telerman, S. B., Liakath-Ali, K., Burdick, J. A., & Watt, F. M. (2016). Mimicking the topography of the epidermal-dermal interface with elastomer substrates. Integrative Biology (United Kingdom), 8(1), 21-29. doi.org/10.1039/c5ib00238a
2. Mobasseri, S. A., Zijl, S., Salameti, V., Walko, G., Stannard, A., Garcia-Manyes, S., & Watt, F. M. (2019). Patterning of human epidermal stem cells on undulating elastomer substrates reflects differences in cell stiffness. Acta Biomaterialia, 87, 256-264. doi.org/10.1016/j.actbio.2019.01.063
3. Langton, A. K., Halai, P., Griffiths, C. E. M., Sherratt, M. J., & Watson, R. E. B. (2016). The impact of intrinsic ageing on the protein composition of the dermal-epidermal junction. Mechanisms of Ageing and Development, 156, 14-16. doi.org/10.1016/j.mad.2016.03.006
4. Murphy, M., Kerr, P., & Grant-Kels, J. M. (2007). The histopathologic spectrum of psoriasis. Clinics in Dermatology, 25(6), 524-528. doi.org/10.1016/j.clindermatol.2007.08.005
5. OECD (2021), Test No. 439: In Vitro Skin Irritation: Reconstructed Human Epidermis Test Method, OECD Guidelines for the Testing of Chemicals, Section 4, OECD Publishing, Paris, doi.org/10.1787/9789264242845-en.
6. Clement, A. L., Moutinho, T. J., & Pins, G. D. (2013). Micropatterned dermal-epidermal regeneration matrices create functional niches that enhance epidermal morphogenesis. Acta Biomaterialia, 9(12), 9474-9484. doi.org/10.1016/j.actbio.2013.08.017
7. Malara, M. M., Blackstone, B. N., Baumann, M. E., Bailey, J. K., Supp, D. M., & Powell, H. M. (2020). Cultured Epithelial Autograft Combined with Micropatterned Dermal Template Forms Rete Ridges In Vivo. Tissue Engineering Part A, ten.tea.2020.0090. doi.org/10.1089/ten.tea.2020.0090
8. Blackstone, B. N., Malara, M. M., Baumann, M. E., McFarland, K. L., Supp, D. M., & Powell, H. M. (2020). Fractional CO2 laser micropatterning of cell-seeded electrospun collagen scaffolds enables rete ridge formation in 3D engineered skin. Acta Biomaterialia, 102, 287-297. doi.org/10.1016/j.actbio.2019.11.051

### Example 2

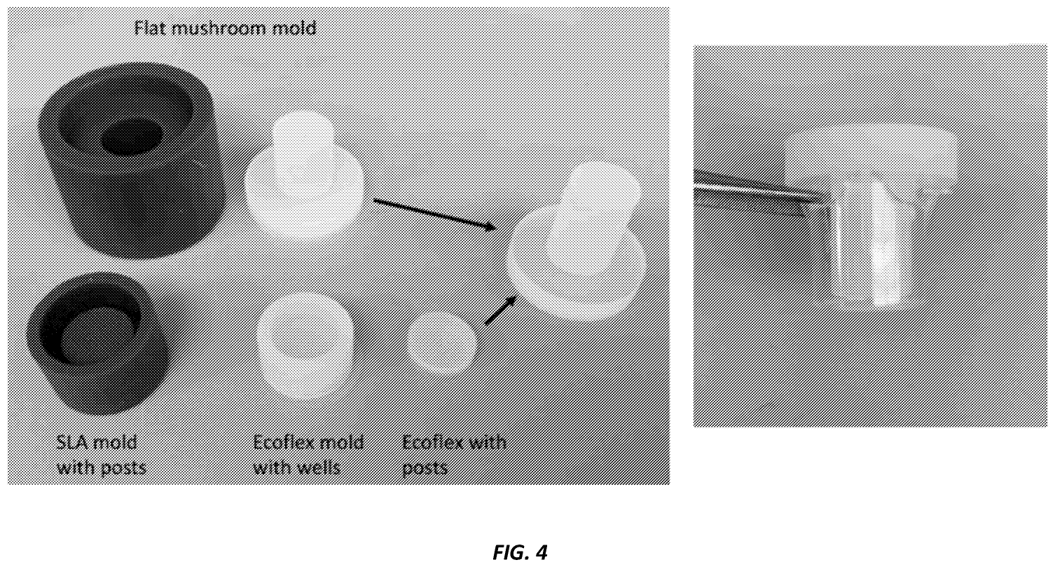

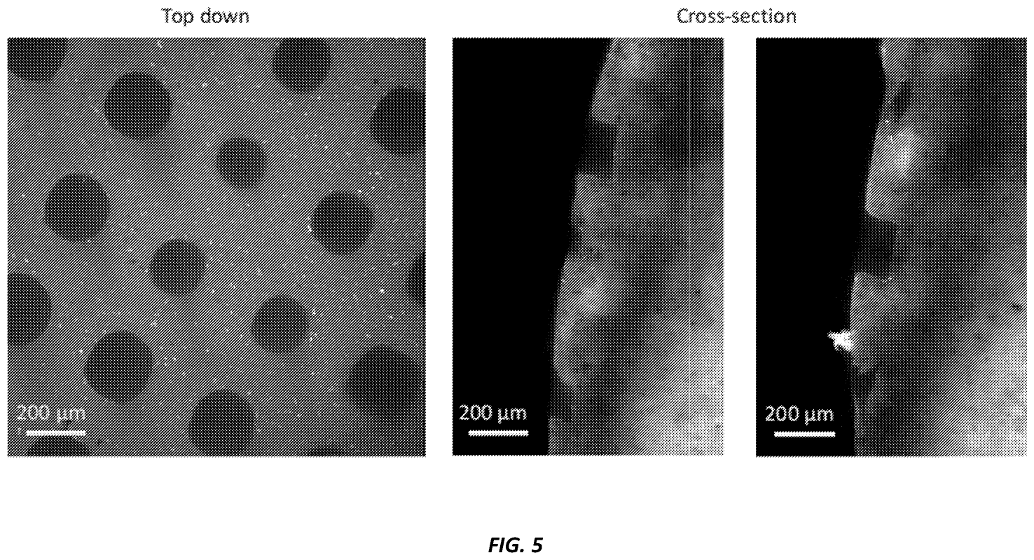

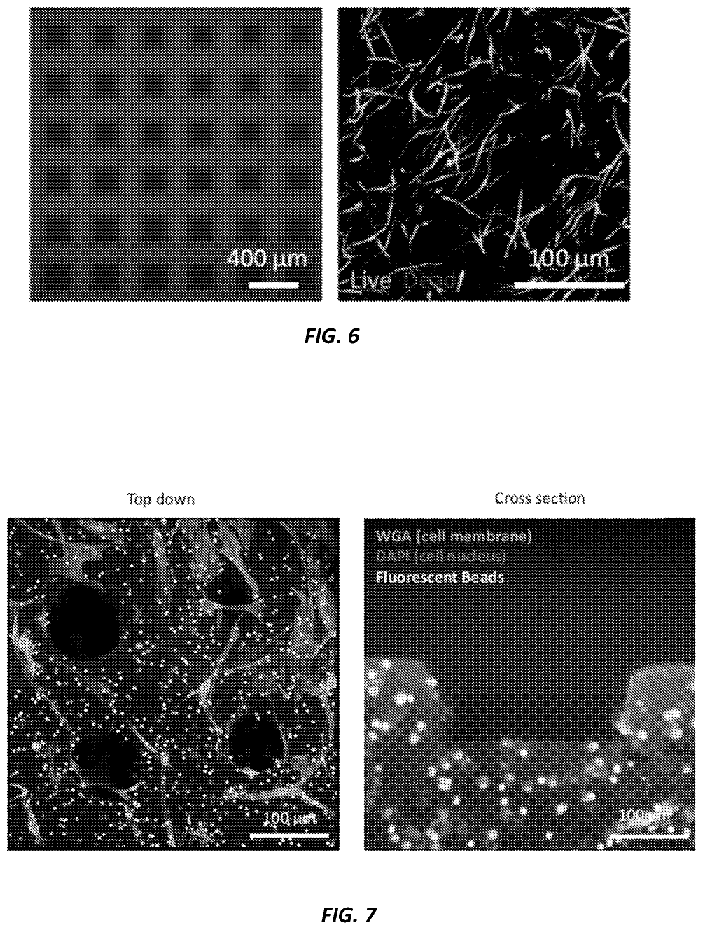

Stamps (FIG. 4) can be used as described herein to microfeatures (e.g., rete pegs) into a gel. FIG. 5 demonstrates the stamping of a gel without cells to product 200 μm diameter×200 μm height peg features. Stamping has been performed with gels comprising gelatin, fibrin, collagen, agarose, and combinations thereof. A gel comprising gelatin and fibrin (gelbrin) provides quick gelling, good stamping, and appropriate levels of fibroblast growth (FIG. 6). The microfeatures are retained when fibroblasts are cultured in the gelbrin (FIG. 7). In comparison, 3 mg/mL collagen gels display more compacting which degrades the microfeatures and a gel comprising both agarose and collagen supports less fibroblast growth.

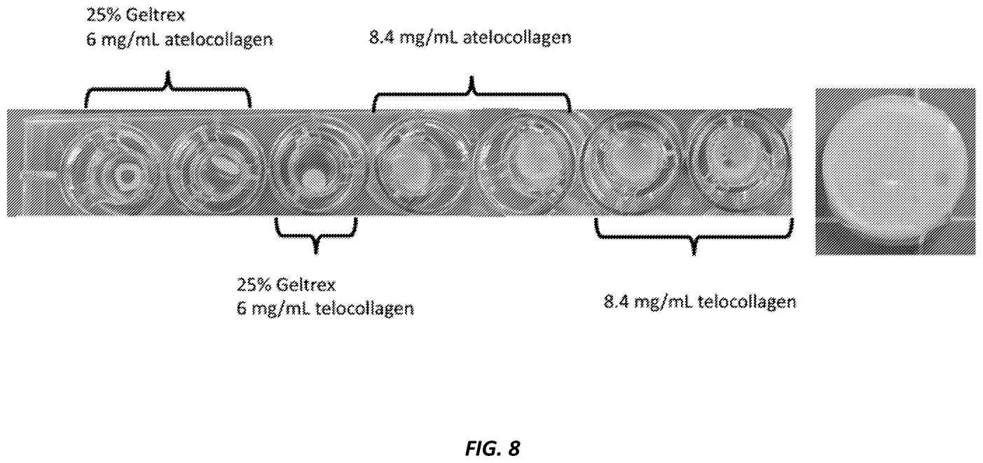

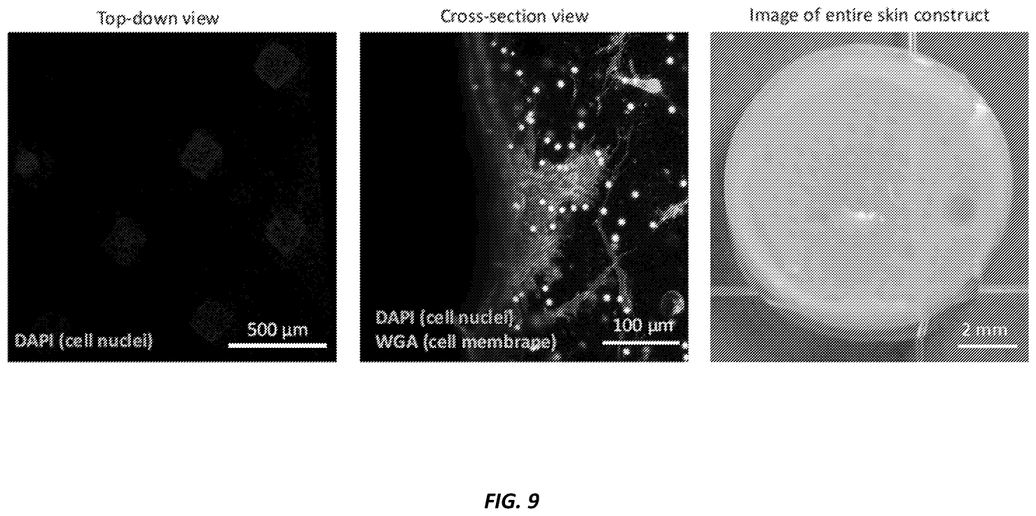

Different forms of collagen were tested. Telocollagen is stiffer and gels more quickly than atelocollagen. Gels comprising high levels (8.4 mg/mL) of either collagen displayed very little contraction as compared to gels with less collagen (FIG. 8). The contraction could be further reduced by crosslinking with TG or adding fibrin (data not shown). The high collagen gels were able to retain the microfeatures for at least 6 days (FIG. 9).

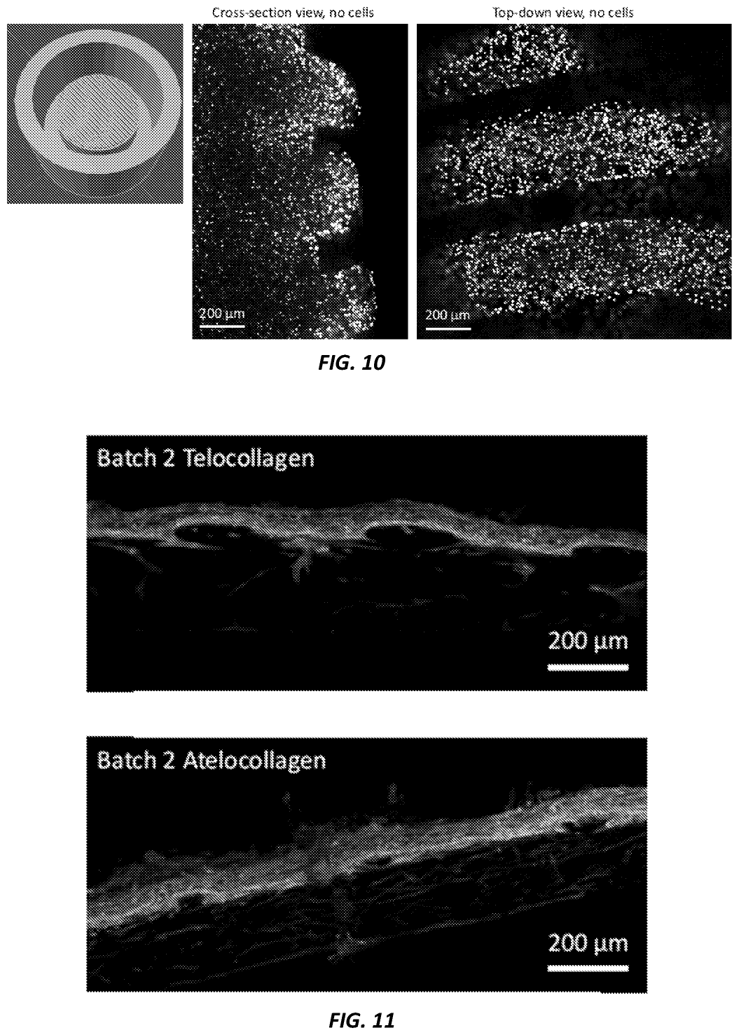

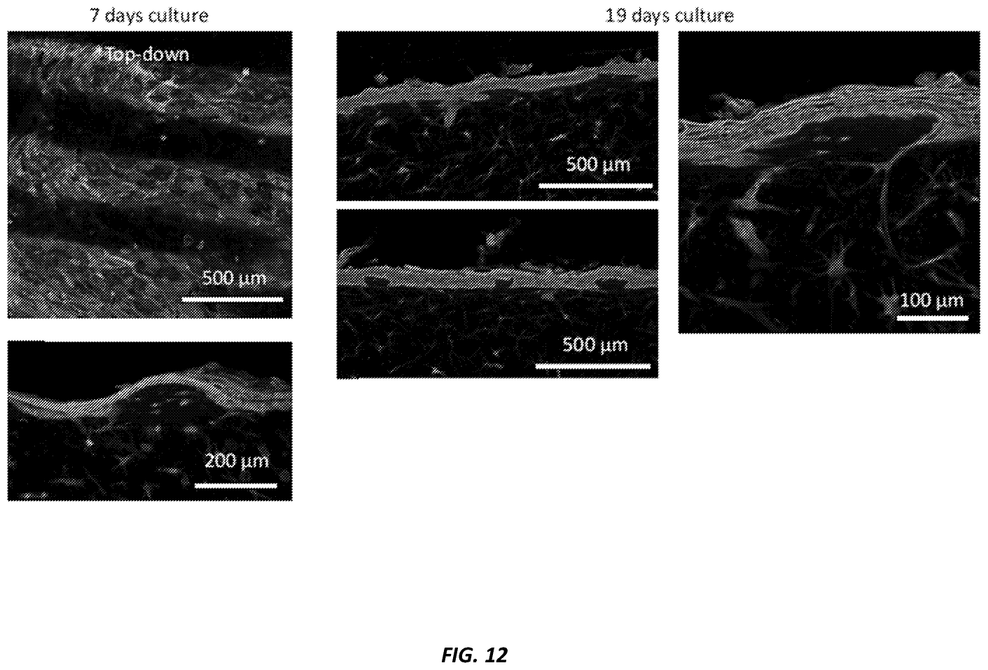

It was demonstrated that microfeatures with various shapes can be introduced into the gels. For example, FIG. 10 shows the results of stamping with a ridge design instead of a post design. High collagen gels (FIG. 11) and high telcollagen+fibrin gels (FIG. 12) were able to retain the ridge microfeatures for at least 19 days.

In selecting a gel, a gel which has adequate strength to preserve the microfeatures and which can also support fibroblast spreading should be selected. Such gels are described herein. In some embodiments, the cell can comprise PEG-collagen or PEG-collagen with collagen methacrylate and/or PEGDA. It is further completed that such features can be achieved by utilizing a multilayered or gradient gel, crosslinking the gel after the fibroblasts have spread, or culturing the fibroblasts below the gel, e.g., below the transwell insert.

It is contemplated herein that the methods and models provided herein can be used to conduct skin irritation testing, mechanical testing (e.g., parallel plate rheology), function as a re-epithelialization model, or as a disease specific skin model.

The approach described herein has advantages over skin organioid models (Lee et al. Nature 2020, which require 140 or more days to culture the organoids via implantation.

Finally, it is noted that the approach described herein has several advantages over 3D bioprinting approaches. First, the present approach is readily scaled up as a larger stamp mold or many molds can be easily 3D printed, while larger bioprints will take more time at the same resolution. Second, the gel material in the present approach must be able to form a gel that can maintain features, while in 3D bioprinting, the material must also be able to retain the features before gelation is complete. Third, stamps are easily made biocompatible, but 3D bioprinting necessarily introduces damaging shear through the nozzle. Fourth, multiple stamps can be applied to different culture containers (e.g., wells or dishes) at the same time, but in 3D bioprinting the first print may be different from the last print and separated by a significant period of time.

### Example 3: Culturing a Skin Model with a Telocollagen and Fibrin Dermis Gel

A common challenge in skin tissue engineering is dermal tissue compaction. In reports of dermal tissue compaction in the literature, skin constructs contract from their original size. For example, a circular skin construct's radius at the end of culture could be half of its starting size, and the thickness of the construct could be reduced to 1/10 of its original size after 2 weeks of culture. This is particularly a challenge when building skin constructs out of collagen, the main component in the human dermis. Because of dermal tissue compaction, it can be difficult to obtain reproducible and repeatable results, and there is a high failure rate in using dermal tissue model. The protocol below, using telocollagen and fibrin as a dermal scaffold, does not exhibit severe tissue compaction, is repeatable, and has a low failure rate.

Fibroblast culture. Primary neonatal dermal fibroblasts from ATCC (PCS-201-010) were used with the low-serum fibroblast media sold by ATCC with phenol red and no antibiotics/antimycotics (PCS-201-030, PCS-201-041, and PCS-999-001). Fibroblasts are passaged using 0.05% Trypsin EDTA, and 5% FBS in PBS without Ca and Mg is used as a trypsin neutralizing solution. Centrifugation is performed for 5 minutes at 150 g. In some embodiments, fibroblasts in experiments are from P4 to P6.

Keratinocyte culture. Primary epidermal neonatal keratinocytes from ATCC (PCS-200-010) were used with CnT-Prime™ media from CELLnTEC, with no phenol red, and no antibiotics/antimycotics. This medium performed better than ATCC's keratinocyte medium, since keratinocytes cultured using ATCC's medium tended to form “fried egg” morphology differentiated cells more frequently in culture. Keratinocytes were passaged using 0.05% Trypsin EDTA, and 5% FBS in PBS without Ca and Mg was utilized as a trypsin neutralizing solution. Centriguation was done for 5 minutes at 150 g. Cells were seeded at about 0.5-1 million cells in a T75. In some embodiments, the keratinocytes cells are used at P2-P3.

An exemplary protocol follows:

One day before keratinocytes are passaged, prep the gels with fibroblasts
1) Materials to make gel
   i. Telocollagen (TeloCol-10, Type I collagen, 10 mg/mL stock, Bovine)
   ii. 10×PBS
   iii. Phenol Red (to visualize collagen pH, ATCC sells a solution of this: PCS-999-001)
   iv. 1M NaOH (to neutralize the collagen)
   v. Fibrinogen, Bovine plasma EMD Millipore 341573 (80 mg/mL stock, stored at −20° C.) Note: Prepare by spreading powder on a 60 mm dish, and pipetting by drop PBS without Ca and Mg to a concentration of 80 mg/mL. Make sure to wet all of the powder. Place dish on a 37° C. heat block for 2 hours or until fully dissolved. Then, aliquot and store at −20° C.
   vi. Thrombin (500 U/mL stock aliquots in sterile water and stored at −20° C.)
   vii. Transwell inserts: Corning, 12 well plate, 12 mm insert, 3 μm pore size, polyester membrane.

A stock solution of 10×PBS with 10× phenol red (330 μM) can be prepared in advance.

### Steps:

1) Place all tubes that will be used for collagen on ice to cool down as much as possible before starting to work with collagen.
2) Fill several (2-3) 15 mL conical tubes or other tubes depending on working volumes with about 10 mL 1×PBS and put this in the ice bucket. This will be used to wash the pipette tips before pipetting collagen.
3) Add 1/10 expected volume with the 10×PBS and phenol red solution, and add estimate for needed 1M NaOH to neutralize the collagen. For example, when adding 2.5 mL of collagen to a total volume of 3 mL, I would add 300 μL of 10×PBS+phenol red, and about 50-55 μL of 1M NaOH. Place this on ice to cool it down before adding collagen.
4) Before pipetting collagen, wash pipette tips 5+times in cold PBS solution. Then pipette telocollagen into the solution with PBS, phenol red, and NaOH. Mix by pipette until the color becomes uniform. If needed, add more NaOH to balance the pH.
5) Keep collagen solution on ice while preparing cells.
6) Passage fibroblasts as usual and count the cells. Add fibroblasts to a new tube at a number that, when added to the collagen, will be 2 million cells/mL. Spin down the cells. Estimated collagen losses due to pipetting and mixing can be accounted for (in the 3 mL case above, there may be losses of as much as 1 mL of solution).
7) Resuspend cells in fibrinogen, so that when added to the collagen solution, it would provide the desired fibrinogen concentration. In some embodiments, between about 5 mg/mL and 10 mg/mL can be used
8) Add thrombin to the collagen solution to reach about 0.25 U/mL to 0.5 U/mL. A higher concentration can be used, but this will cause the solution to gel much more quickly.
9) Add the fibrinogen with cells to the collagen with thrombin solution, and mix quickly. (Wash pipette tip in cold PBS before mixing.) Once sufficiently mixed, add about 250-275 μL to the bottom of the transwell insert (less can be used for a thinner sample, but since the solution is viscous, it may not fully cover the bottom of the transwell insert at lower amounts).
10) Because of losses of the collagen solution, one of these target 3 mL solutions should be sufficient for about 4-6 transwell inserts.
   i. 300 μL 10×PBS and 10× phenol red (330 μM)
   ii. 52.5 μL 1M NaOH
   iii. 2.5 mL telocollagen at 10 mg/mL
   iv. 125-250 μL of fibrinogen+cells
11) Note: when mixing the collagen solution by pipette, try not to introduce bubbles. If bubbles enter the collagen solution, either centrifuge at 4° C. for a few minutes to remove bubbles, or leave the collagen on ice for 15 minutes to let the bubbles rise to the surface.
12) Allow samples to gel for 60-90 minutes at 37° C. in the incubator, and then apply media on the inside of the inserts. Alternatively, the media can be added on the outside of the transwell inserts before this gelling step or after the step.
13) Incubate for 1 day and add keratinocytes the next day.

Passage the keratinocytes and add to the top of the gels. 1 day later, Passage keratinocytes using 0.05% trypsin-EDTA at 80-90% confluence. Exchange media in the gels to CnT-FTAL™, the media used from CELLnTEC to coculture keratinocytes and fibroblasts. Count keratinocytes, and add 400,000 cells to each of the transwell inserts.

Tissue culture after gels have been prepped. Culture was done using CELLnTEC's CnT-FTAL™ as medium, with exchanges on Monday, Wednesday, and Friday (or every other day). 2 mL of media was used below the inserts and 0.5 mL inside the inserts. 4 days after keratinocytes were added, the samples were lifted to the air-liquid interface using autoclaved rubber spacers. The silicone rubber is from McMaster Carr, High temperature silicone rubber sheets, 2.5 mm thick, for example: 3788T35. Circles are cut from the spacers in the pattern of a 12 well plate using a ⅞ inch circular hole punch, for example: 3427A22. At the air liquid interface on the spacers, 2.5 mL of media was added below the inserts. Culture is continued for an additional 2 weeks before fixing the samples.

Staining. Samples were fixed at room temperature for 1-2 hours by pipetting 500 μL of media on top of the samples. Samples were washed in PBS three times, with 500 μL on top, and 2 mL below each samples, 30 minutes each wash.

Following the fixation period, scalpel blades were used to cut the samples out of the transwell inserts and then the samples were cut into thin rectangles. See FIG. 15 for a diagram of how the samples were cut. Samples were these into quarters (one half can be retained for additional stains as needed). Then, about 2 mm wide samples of the sample are cut with the scalpel. These are stored in PBS until ready for staining. Image samples close to the middle 50% of the transwell insert were usually chosen.

Wheat germ agglutinin+DAPI can be added immediately (no blocking/permeabilization required). Stocks of both of these at 1 mg/mL, and I add wheat germ agglutinin at a dilution of 1:200, and DAPI at a dilution of 1:5,000 can be used.

For antibody stains the following procotol was used. These times were used for whole-mount samples, and it is contemplated herein that the times can be reduced as sample size reduces.
1) Permeabilize with 0.5% Triton X-100 for 30 minutes at room temperature
2) Block with 5% Donkey serum in PBS with 0.1% Triton X-100 overnight
3) Primary antibody in 5% Donkey serum, 0.1% Triton X-100, 1×PBS, for 2-3 days at room temperature
4) Wash 3× in 1×PBS at room temperature, 2 hours each wash
5) Secondary antibodies in 5% Donkey serum, 0.1% Triton X-100, 1×PBS, include ActinRed 555 Ready Probes reagent, for 1-2 days at room temperature
6) Add DAPI for 2 hours or overnight before imaging
7) Wash 3× in 1×PBS at room temperature, 2 hours each wash
8) Image

### Stains and Antibodies

Wheat germ agglutinin 488, Invitrogen, ThermoFisher catalogue number W11261. I prepare a stock solution at 1 mg/mL in PBS, and then dilute to 5 μg/mL in the staining solution in PBS. Stock can be stored at −20° C.

DAPI: A stock solution of 1 mg/mL can be diluted by 1:5,000 in the same wheat germ agglutinin solution.

ActinRed 555 Ready Probes (rhodamine phalloidin) Invitrogen, Thermofisher catalogue number R37112. 2 drops per mL can be added to the secondary antibodies solution. This overlaps well with wheat germ agglutinin.

Primary Antibodies (from Abcam)
1) Ab7800, mouse monoclonal to cytokeratin 14
2) Ab76318 rabbit monoclonal to cytokeratin 10
3) Ab218395 mouse monoclonal to filaggrin
4) Ab6311 mouse monoclonal to collagen IV
5) Ab11575 rabbit polyclonal to laminin
   All primaries were diluted 1:200 in 5% donkey serum, 0.1% Triton X-100, and 1×PBS.
   Secondary Antibodies (from Abcam)
1) Ab150075 Donkey Anti-Rabbit IgG H&L AlexaFluor 647
2) Ab150105 Donkey Anti-Mouse IgG H&L Alexa Fluor 488
   All secondary antibodies were diluted 1:500 in 5% donkey serum, 0.1% Triton X-100, and 1×PBS.

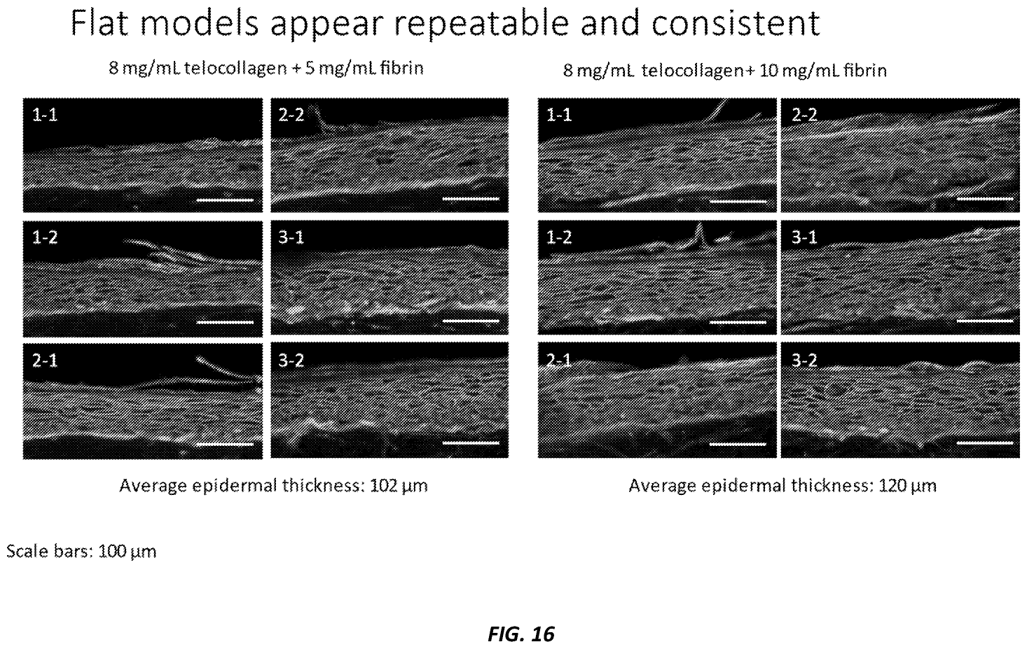

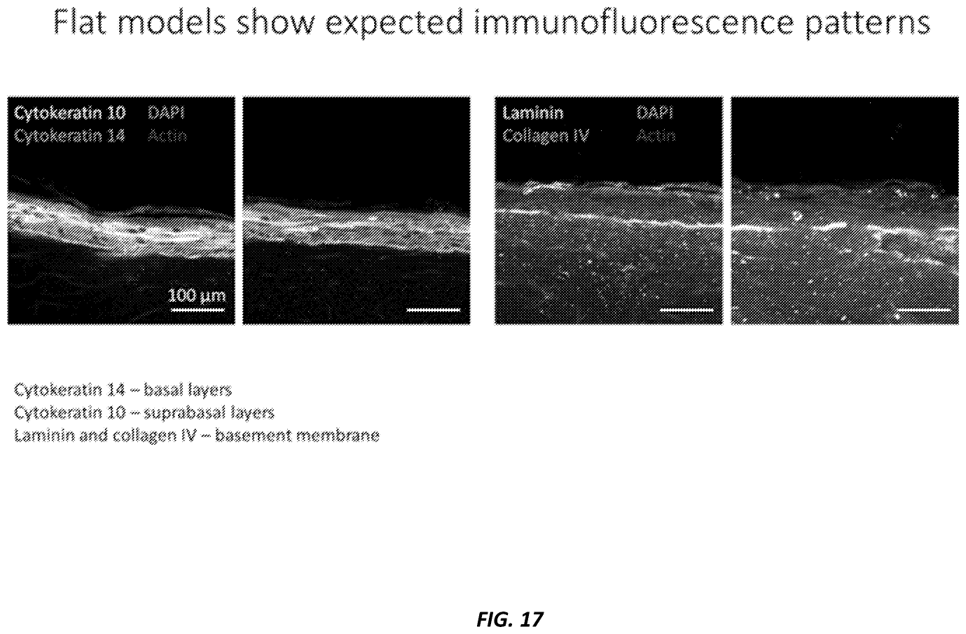

FIGS. 16 and 17 demonstrate that the flat model cultures prepared according to the above protocol display repeatable and consistent morphology (FIG. 16) with the expected immunofluorescence patterns (FIG. 17).

### Example 4: Stamping Method for Human Skin Model with Rete Pegs

Undulations between the epidermis and dermis in skin, known as rete pegs, are important to the mechanical properties of skin. Rete pegs flatten with age and sun exposure, and can change structure in different skin disorders, such as psoriasis. Despite the importance of rete pegs in human skin, most biofabricated skin models have a flat interface between the epidermal layer and the dermis layer. Described herein is a method for fabricating human skin models with control of a variety of rete peg structures. In some embodiments, the method uses 3D-printed molds to stamp rete peg-like features into human skin models. While keratinocytes degraded features stamped into atelocollagen-based gels, telocollagen gels had lasting features through 14 days of cell culture. To better preserve the features, the telocollagen-based gels were crosslinked, leading to minimal changes in the feature sizes overtime. In this model, expected epidermal markers were observed, including cytokeratins, basement membrane proteins, and proliferation markers. It is also demonstrated that two keratinocyte cell types, one from a neonatal donor and one from an adult diabetic donor, were both compatible with this model. Finally, this model was tested using an irritation test, and the epidermis developed a functional barrier. It is contemplated that this skin model can be used as a disease model or a mechanical model for human skin.

### INTRODUCTION

The interface between the epidermis and dermis layers in human skin undulates. These undulations are known as rete pegs or rete ridges. Typically on the length-scale of 50-400 μm¹⁻³, rete pegs contribute to the elasticity of skin⁴ and adhesion between the dermis and epidermis, potentially because of the increased surface area between the two layers⁵. It has also been shown that populations of epidermal stem cells may be biased in different regions of the rete pegs (“hills” or “valleys”)^{6,7}. Rete pegs are known to flatten with age and sun exposure, decreasing biomechanical function^{4,8,9}, and rete pegs can be elongated in psoriatic lesions¹⁰. Moreover, burn wound victims who undergo grafts of keratinocyte sheets without rete pegs have increased likelihood of blistering⁵. Because of the importance of rete pegs in skin biology and the variety of rete peg structures observed in patients, it would be valuable to have a skin model that can reproduce a variety of rete peg geometries.

Many traditional skin models and recent bioprinted skin models have a flat interface between the dermis and the epidermis¹¹⁻¹⁵. Several papers have fabricated rete peg structures in human skin models. Microfabrication-based approaches have been used^{1,2,16}, but these approaches typically only contain one cell type, which may limit the production of basement membrane proteins, important for skin elasticity⁸. Rete peg structures have also been generated by laser ablation, however, this can damage the cells in the scaffold, and the authors were limited to two different types of structures they could generate^{3,17}. GelMA and PEGDA-based scaffolds have also been used to fabricate rete peg structures, but the authors did not demonstrate a multilayered epidermis or deposition of basement membrane proteins when samples were cultured in vitro¹⁸.

Described herein is a method to generate a variety of rete peg-like structures in a human skin model and evaluate the stability of the rete peg structures, the production of basement membrane proteins and other epidermal markers, and the barrier properties of the model. The method uses 3D-printed molds to stamp rete peg-like structures into skin models. Gels containing telocollagen are more robust to degradation by keratinocytes. Additionally, crosslinking gels with EDC yields more stable structures. The epidermal cells express cytokeratins, basement membrane proteins, and proliferation markers in expected patterns. This approach can generate skin models with rete pegs using keratinocytes from both neonatal donors and adult diabetic donors. The use of these skin models in an irritation test was demonstrated, showing that the skin models have barrier function.

### Results

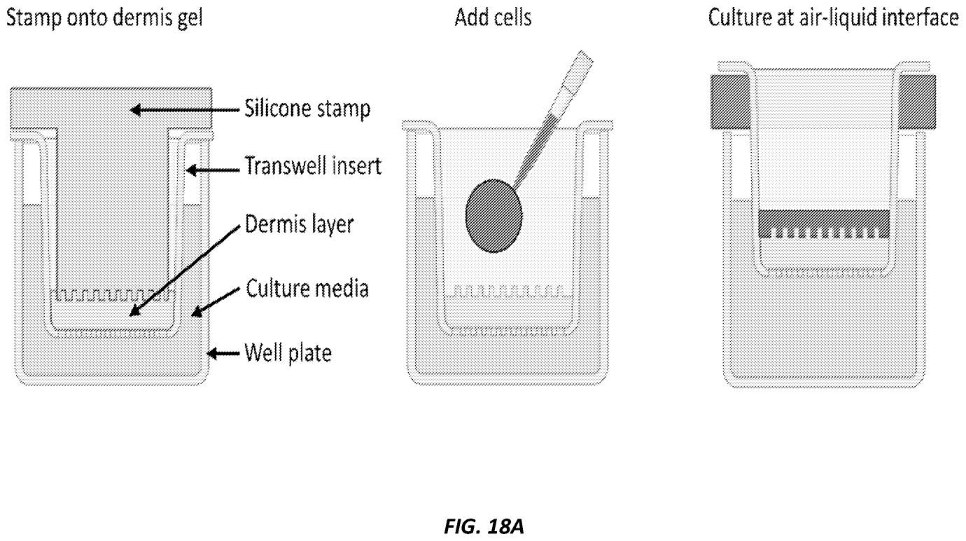

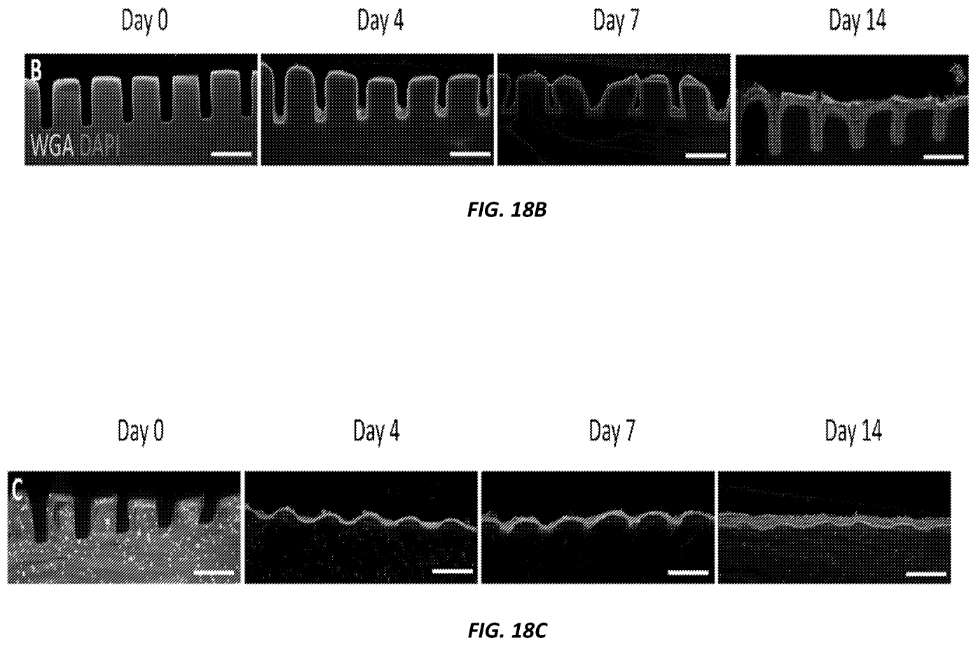

Described herein is a stamping method to generate rete peg-like human skin models (FIGS. 18A-18C). First, silicone stamps were prepared from 3D-printed molds. Second, a dermis gel layer was prepared, containing telocollagen and fibrinogen. Third, the stamps were applied into the dermis gel layer and the collagen and fibrinogen allowed to gel at 37° C. On removal of the stamps, a gel was generated with undulating features, similar to the rete pegs in skin. Human skin cells were added to this model either encapsulated in the gel or by seeding on top of the gel. In all of the models in this Example, primary human keratinocytes were seeded to the top of the gel and these cells were cultured for four days submerged in media and ten additional days at the air-liquid interface to allow them to form a stratified epidermis. Long ridges rather than pegs as in real human skin for this study because the ridges are much easier to consistently and repeatably image by cross-section (FIGS. 18D-18C).

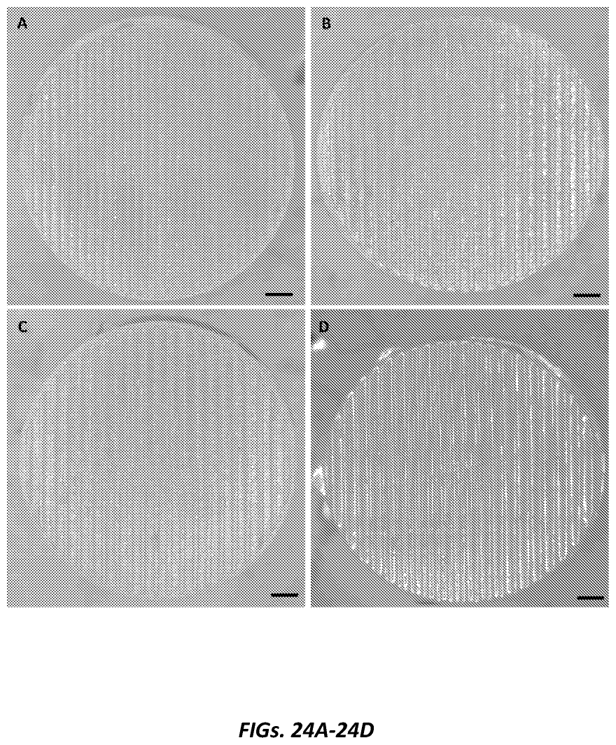

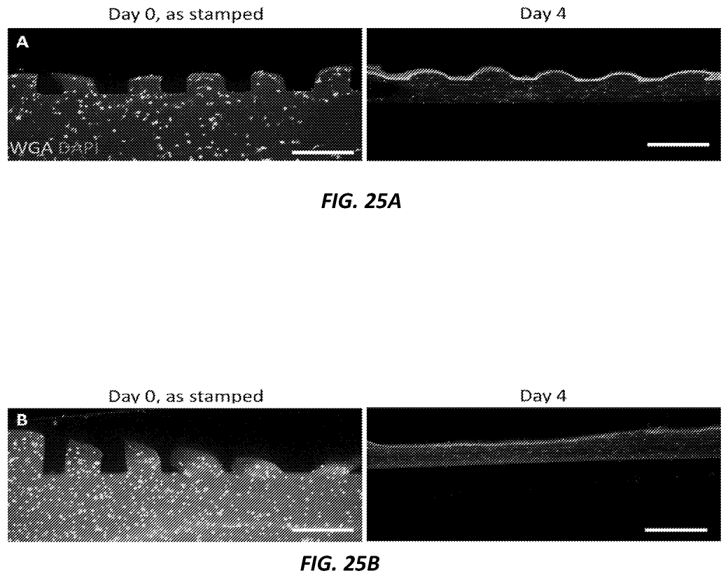

Silicone stamps with feature sizes of 200 μm, 300 μm, 400 μm, and 500 μm were fabricated (FIGS. 24A-24D). First, fibroblasts were encapsulated in a collagen and fibrin gel stamped with the 400 μm silicone features and keratinocytes seeded on top. It was found that keratinocytes quickly degraded the features in stamped skin models made from atelocollagen (7.9 mg/mL) and fibrin (4.6 mg/mL) in four days (FIGS. 25A-25B). However, when features were made using telocollagen (7.9 mg/mL) and fibrin (4.6 mg/mL), the features were rounded slightly, but still were clearly visible after four days of culture (FIGS. 25A-24B). This finding led to exploration of the use of telocollagen in skin models cultured for longer times.

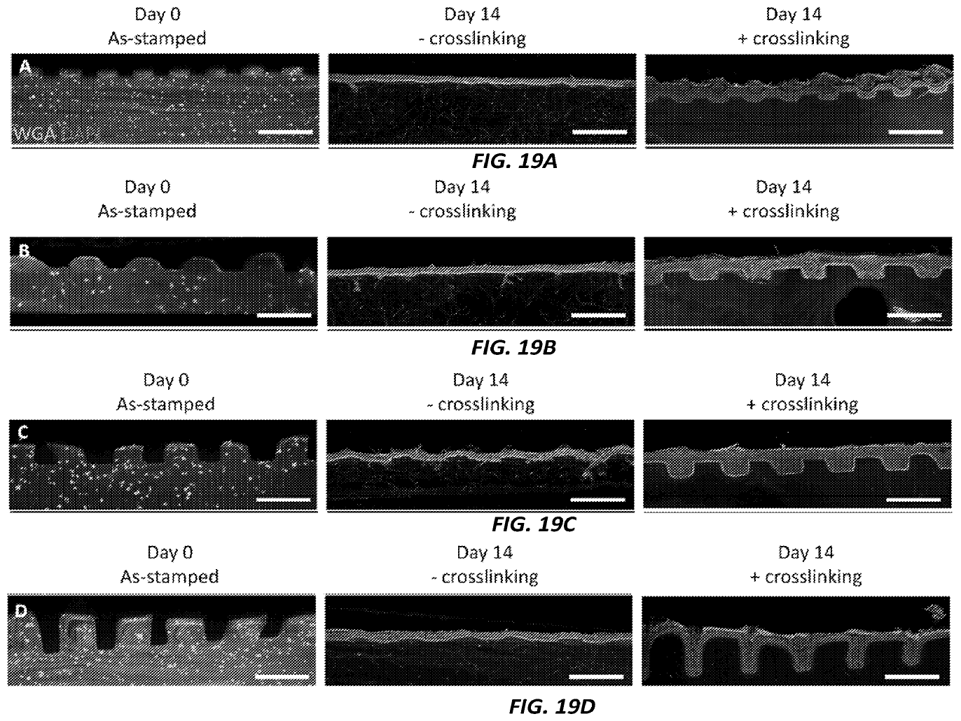

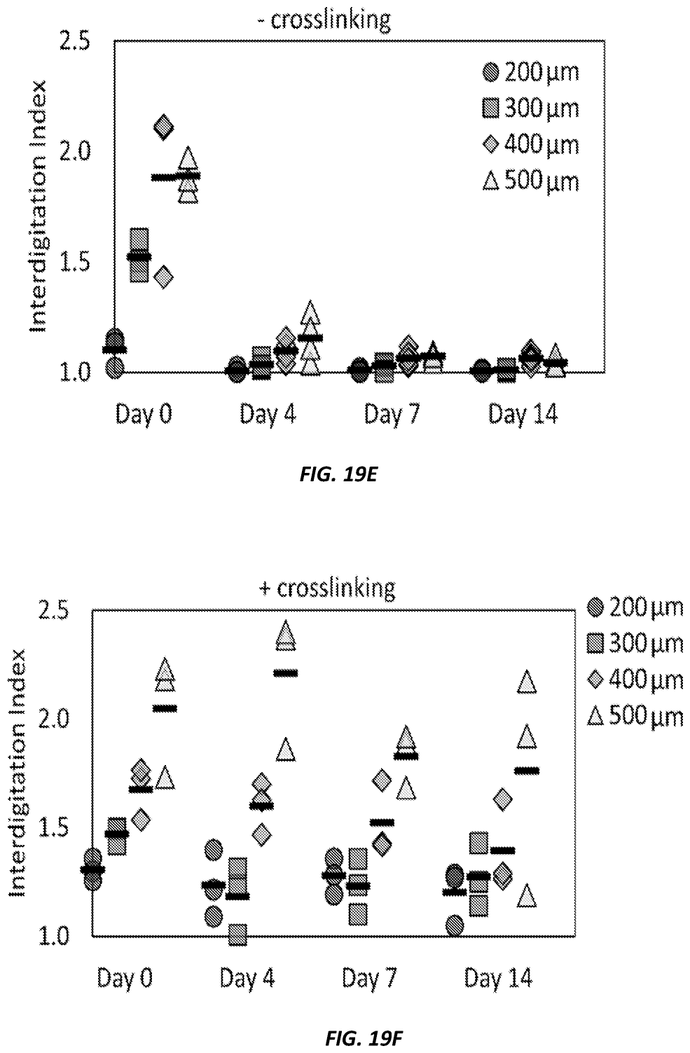

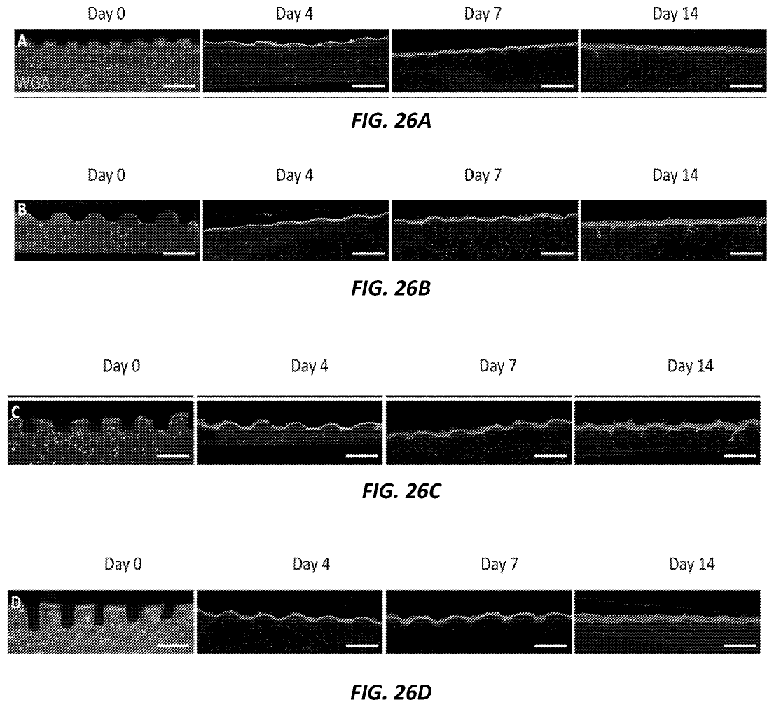

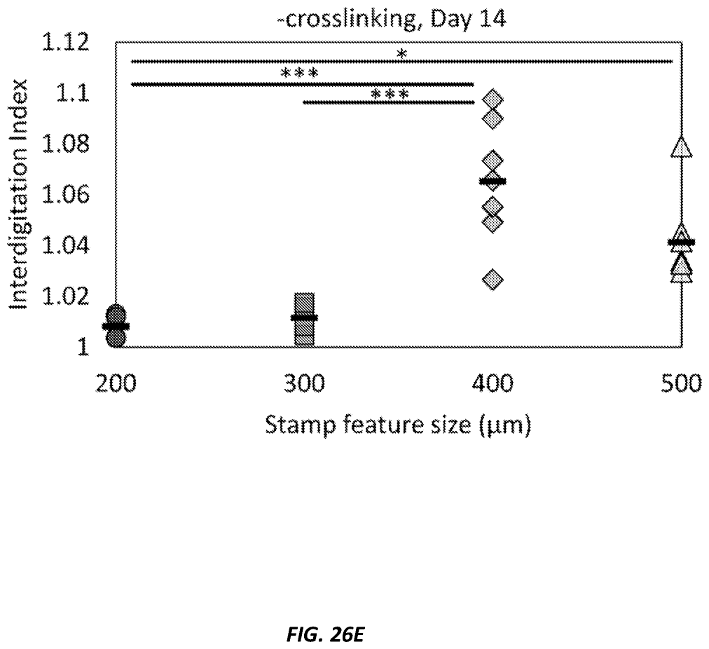

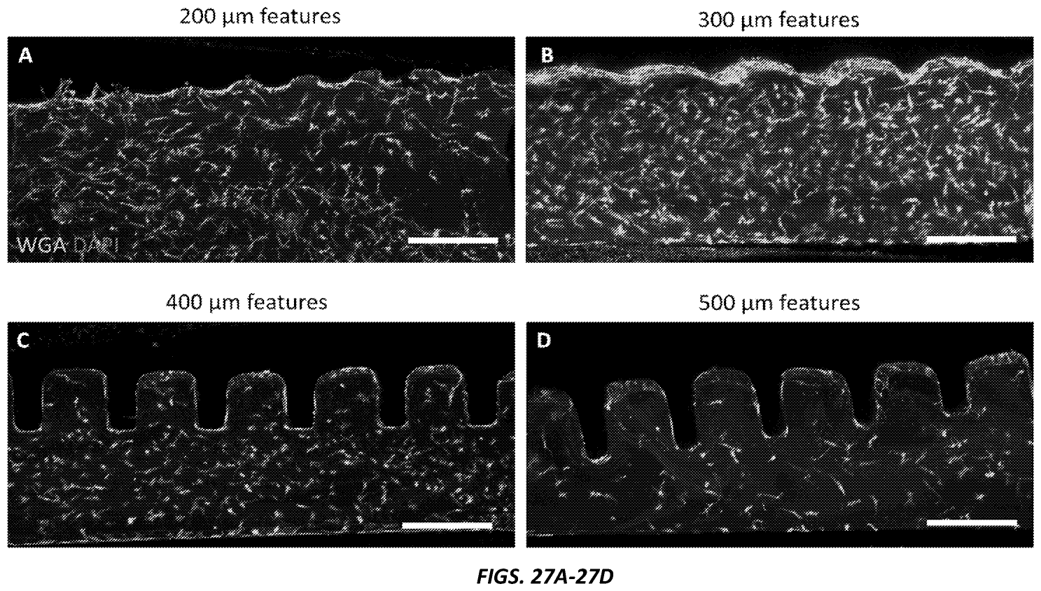

When the telocollagen and fibrin samples with encapsulated fibroblasts were cultured for 14 days to allow the keratinocytes to form a multilayered epidermis, it was found that the structures diminished over time (FIGS. 19A-19F and 26A-26E). Samples were cultured that were stamped with features of 200 μm, 300 μm, 400 μm, and 500 μm. While it was found that the smaller feature sizes of 200 and 300 μm degraded to a roughly flat surface, the 400 and 500 μm stamps yielded visible undulations that persisted, although they were much smaller than at day 0. These features were quantified using a metric known as the interdigitation index¹⁹, the ratio of the dermo-epidermal barrier length across an image and the straight-line length. It was found that the interdigitation index dropped rapidly over the two weeks of culture, but that the 400 and 500 μm stamps were statistically significantly greater than the 200 and 300 μm stamps after 14 days (FIGS. 26A-26E). Interestingly, when fibroblasts were encapsulated in stamped gels but did not include keratinocytes, the same degradation patterns were not observed after 14 days of culture (FIGS. 27A-27D). This indicates that the keratinocytes are the main drivers of the observed degradation, not the encapsulated fibroblasts.

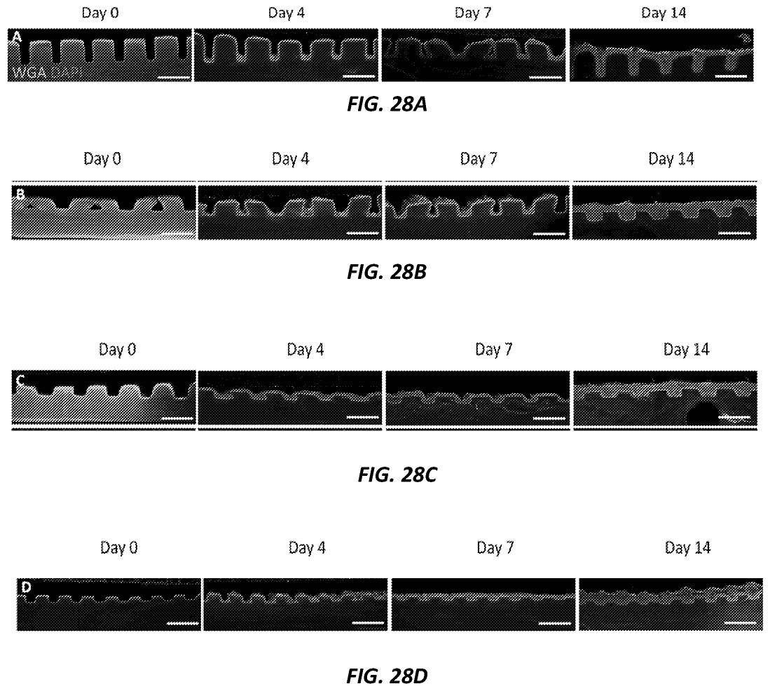

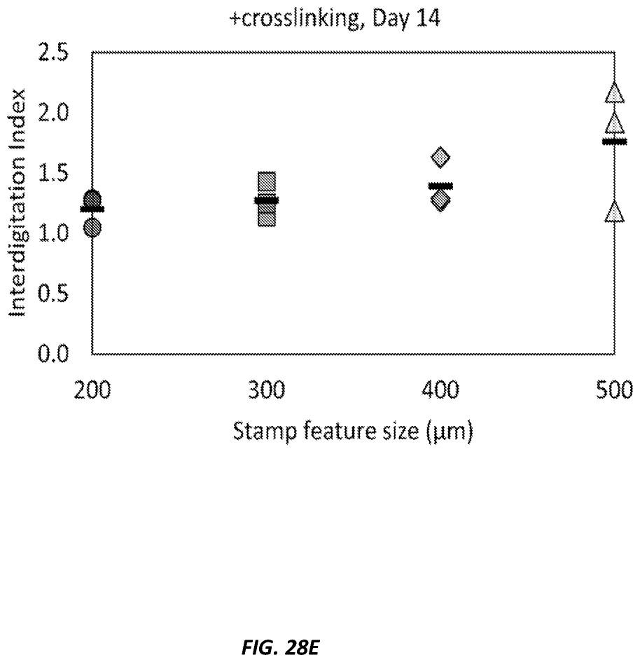

To improve the stability of the features, the stamped telocollagen and fibrin gel were crosslinked with 1-Ethyl-3-(3-dimethylaminopropyl)carbodiimide (EDC). EDC has been used in prior literature involving human skin models to stabilize collagen gels^{1-3,16,17}. In order to use EDC crosslinking with the present models, the stamping protocol was modified. Stamped telocollagen (7.9 mg/mL) and fibrin (4.6 mg/mL) was generated without encapsulated fibroblasts, and then crosslinked gels with 2.5 mL of 14 mg/mL EDC in 100% ethanol per well for 24 hours. Because the fibroblasts were not encapsulated, the fibroblasts were seeded on top of the crosslinked gels and allowed to grow for 4 days before adding keratinocytes. Following image quantification using the interdigitation index, it was found that the crosslinked gels retained features significantly better than the gels that were not crosslinked, and that the original features were not substantially changed throughout the 14-day culture period (FIGS. 19A-19E and 28A-28E).

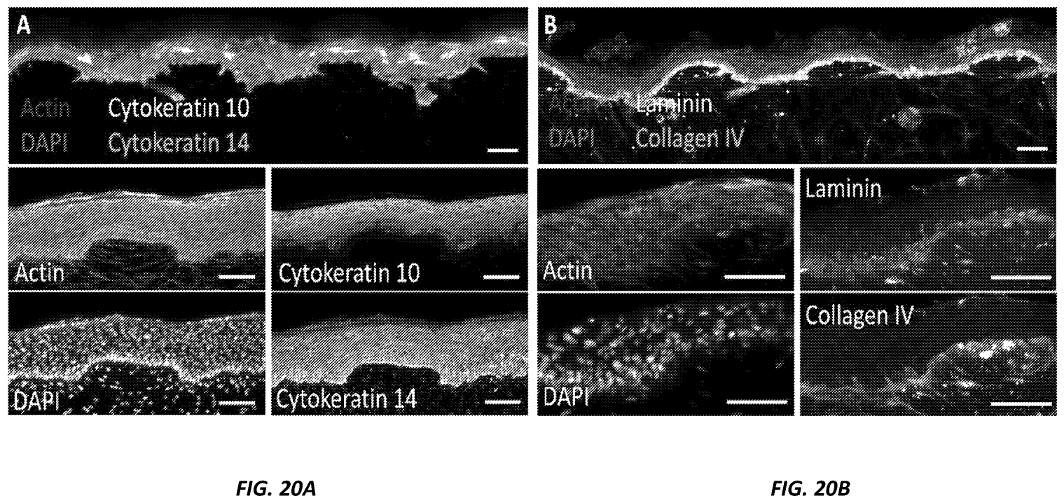

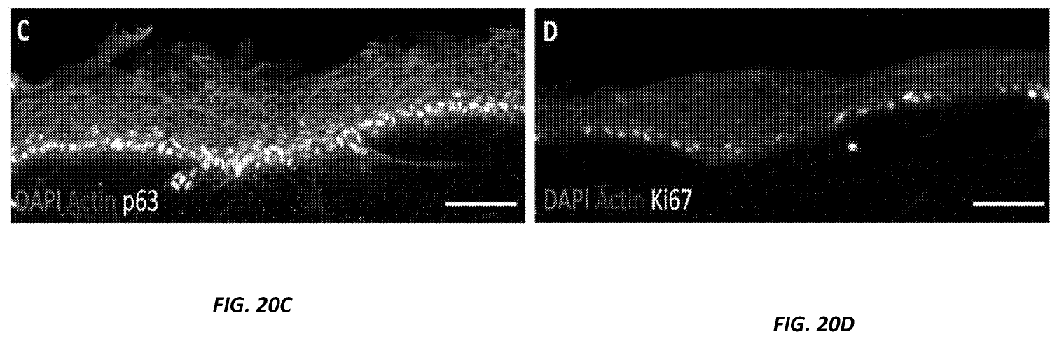

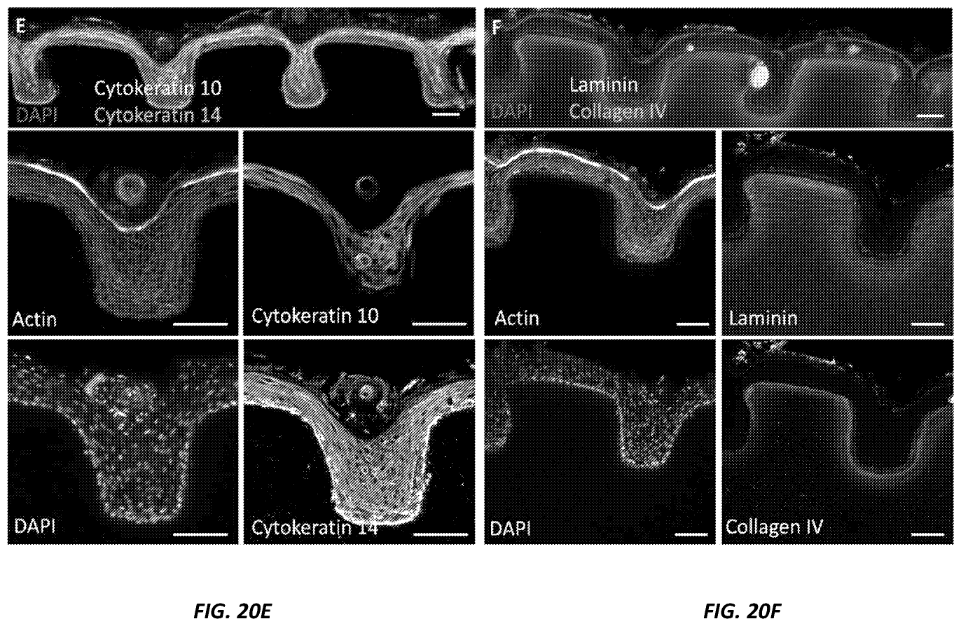

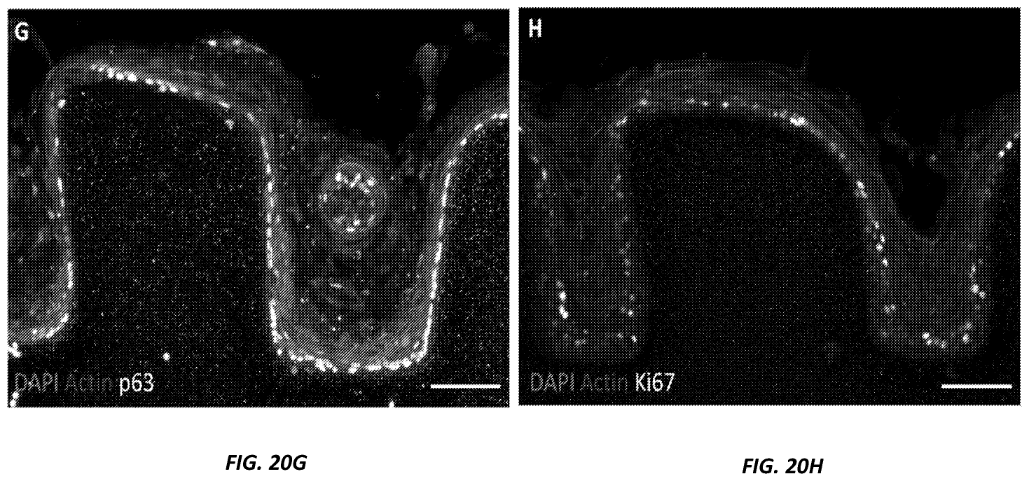

The EDC-crosslinked and not crosslinked skin models were also stained to check if the skin models were expressing expected proteins (FIGS. 20A-20H). The models were stained for cytokeratin 14, which is expected to be expressed in the lower layers of the epidermis, and cytokeratin 10, which is expected to be expressed in the upper layers of the epidermis (FIGS. 20A and 20E). For both the crosslinked and not crosslinked samples, the results match expectations. The models also stained for basement membrane proteins laminin and collagen IV (FIGS. 20B and 20F) and expression was found at the boundary between the dermis and epidermis. For the crosslinked case, the laminin and collagen IV could be produced by both the keratinocytes and the fibroblasts that were seeded on top of the crosslinked gel. The models were also stained for Ki67, a proliferation marker, and p63, a putative epidermal stem cell marker²⁰, and it was found that these were both expressed in the basal layer of the epidermis (FIGS. 20C, 20D, 20E, and 20H). This matches the expectation that the basal layer cells are the proliferative cells to generate the multilayered structures. Overall, qualitative differences were not observed between the EDC crosslinked and not crosslinked skin models in terms of protein expression.

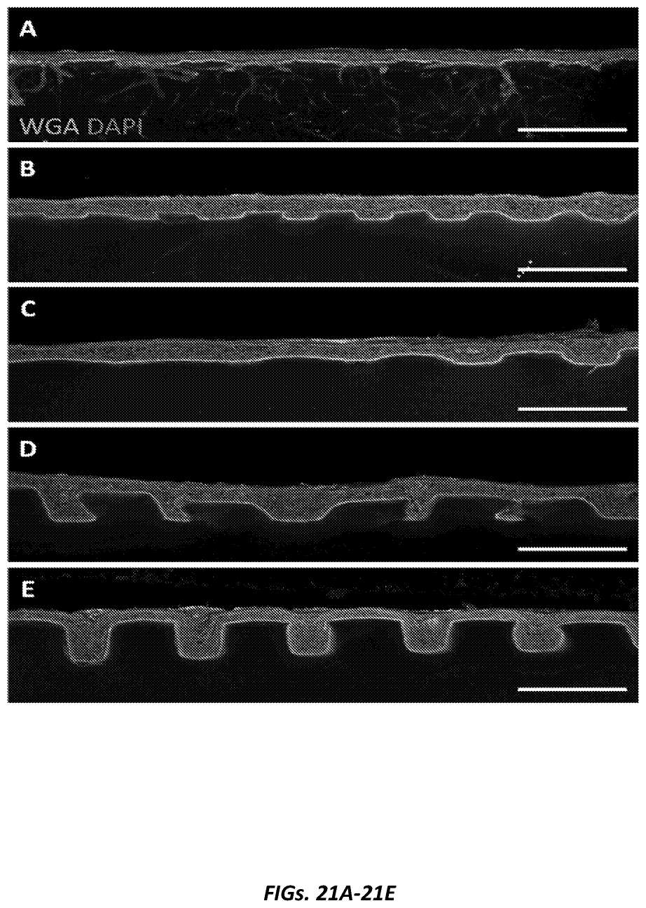

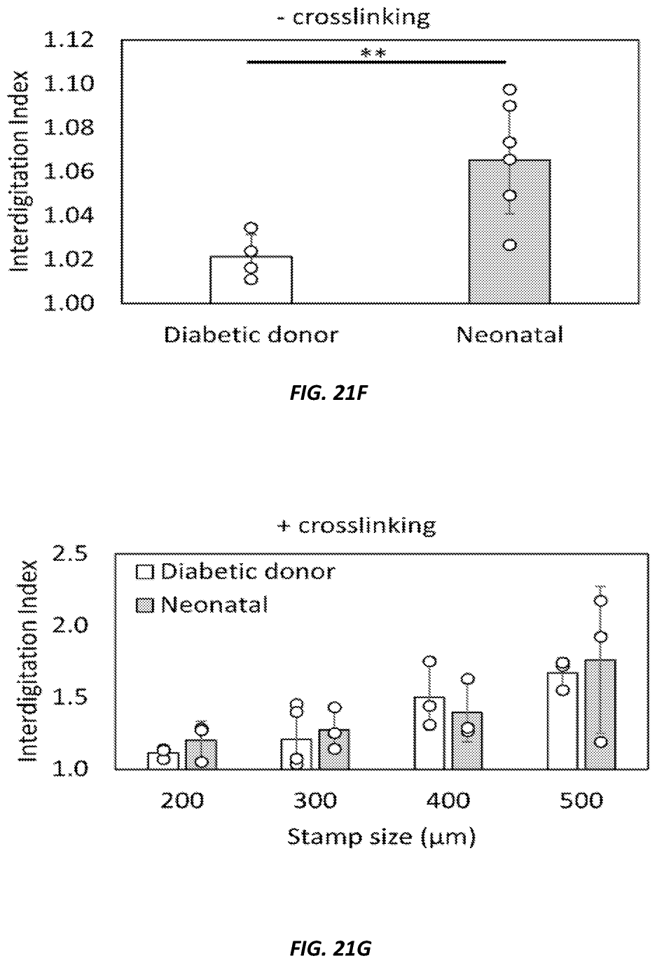

While the data in FIGS. 18-20 use keratinocytes from neonatal donors, it was also wanted to see if this skin model was compatible with other donor cells. The skin model was tested using cells from an adult diabetic patient. These cells were cultured on the 400 μm stamped skin model without crosslinking, and crosslinked gels with stamped features of 200 μm, 300 μm, 400 μm, and 500 μm. Ti was found that in all cases, the adult diabetic donor keratinocytes developed a multilayered confluent epidermis (FIGS. 21A-21G). It was found that the interdigitation index for the not crosslinked samples was smaller than the value for neonatal keratinocytes (FIG. 21F). This could indicate that the adult diabetic donor keratinocytes were degrading the telocollagen-fibrin scaffolds more than the neonatal keratinocytes. When adult diabetic donor keratinocytes were cultured on the crosslinked scaffolds, however, the measured interdigitation indices were not significantly different from the neonatal keratinocytes (FIG. 21G).

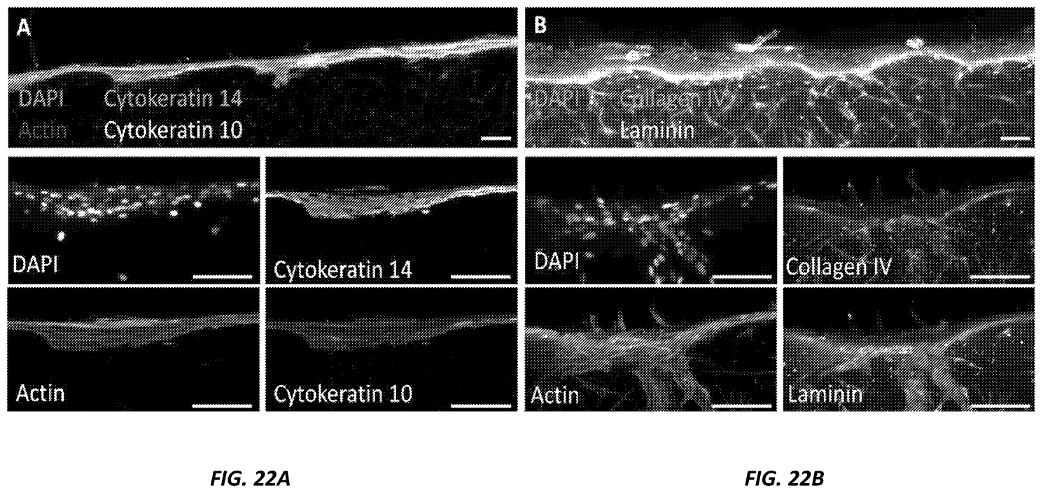

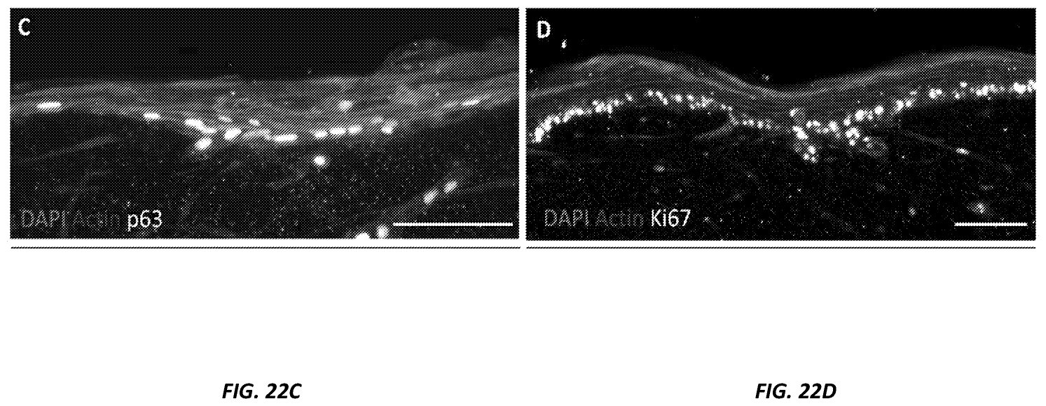

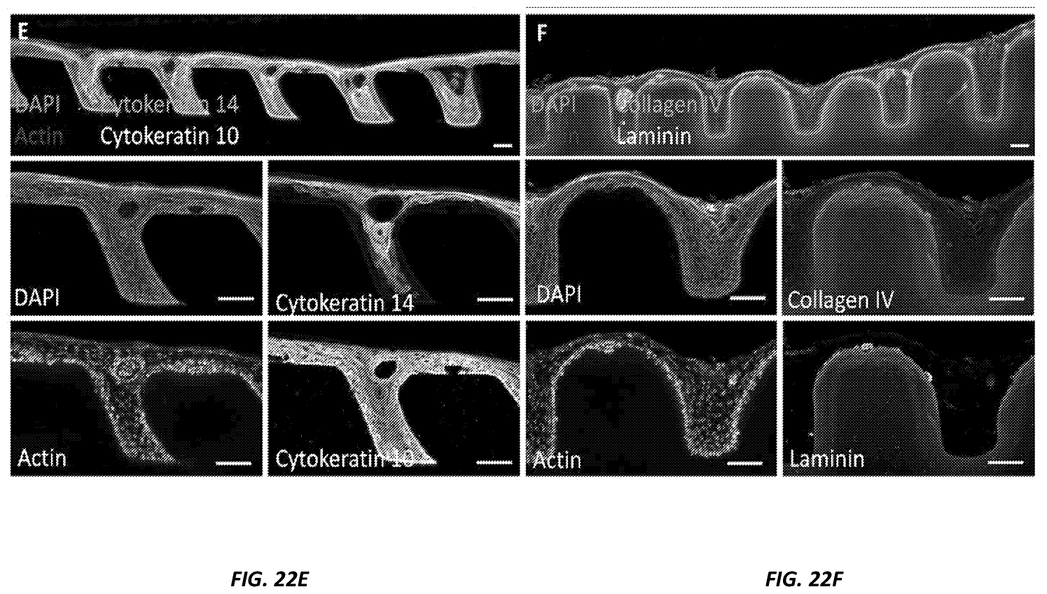

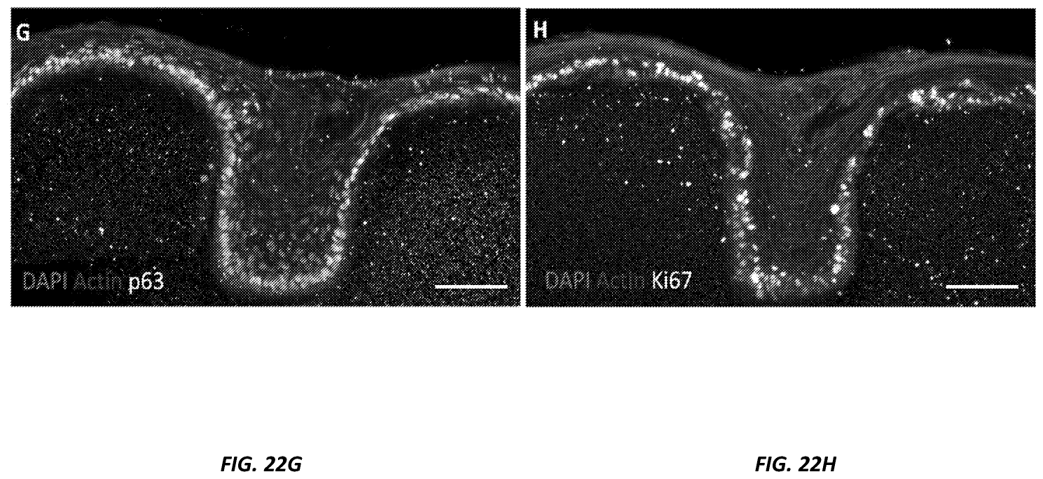

The adult diabetic donor keratinocyte samples using the same stains that were evaluated in neonatal keratinocytes (FIGS. 22A-22H). Similar patterns were observed in cytokeratin expression, basement membrane protein expression, and basal layer keratinocyte proliferation.

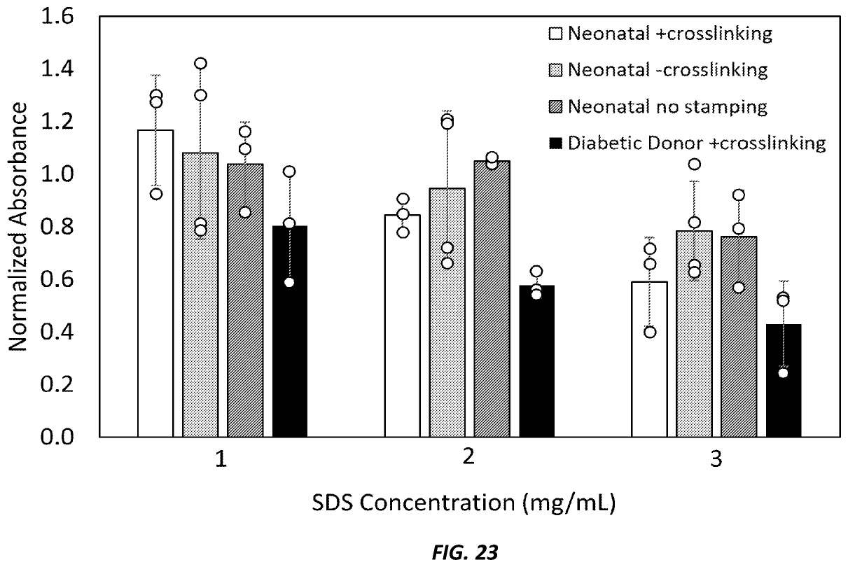

One potential use-case for these skin models is as an irritation model for pharmaceuticals, cosmetics, or occupational health studies. To evaluate the use of these stamped models in irritation tests, a common test method²¹ was adapted. Sodium dodecyl sulfate (SDS) was applied to the top of these samples after 14 days of culture and incubated at 37° C. for 18 hours. After, an MTT assay was used to measure the cell viability. If the epidermal barrier protects the sample from the SDS, the sample will demonstrate high viability. If the SDS penetrates through the epidermal barrier, the sample will not be viable and give a low signal in the MTT assay. It was found that stamped, crosslinked, and flat skin models had similar values after the MTT assay (FIG. 23). The model prepared from adult diabetic donor keratinocytes showed slightly lower viability at each concentration. Moreover, the standard for this test method is that the normalized viability reaches 50% between 1-3 mg/mL of SDS added to the samples²¹. The samples here met this standard, as the 3 mg/mL neonatal samples have absorbance values greater than 50% of the negative control, and the diabetic sample with 2 mg/mL SDS has absorbance value greater than 50% of the negative control. This shows that an epidermal barrier forms in these skin models and establishes the use of these skin models in other irritation tests.

### DISCUSSION

Described herein is a stamping approach to generate rete peg-like structures in skin models using neonatal keratinocytes and adult diabetic donor keratinocytes. It was found that while the keratinocytes partially degraded the telocollagen-fibrin based scaffolds, there were lasting features after 14 days of culture. Significantly less degradation was found using EDC crosslinked scaffolds than telocollagen and fibrin scaffolds without crosslinking. Compared to neonatal keratinocytes, the adult diabetic donor cells showed greater degradation of the scaffolds that were not crosslinked, but similar features in the EDC-crosslinked scaffolds. Finally, the samples were tested with an irritation test and it was found that the samples meet barrier function expectations.

The embodiments described in this Example utilized two approaches for generating a two-layer skin model: samples that are not crosslinked, and samples that are crosslinked. The advantage of the crosslinked model is that the stamped features are more stable over time than in the not crosslinked model. However, the model that does not involve crosslinking with EDC enables encapsulation of fibroblasts, and potentially other cell types, in the dermis layer. For studies that require cells in the dermis layer, the approach without crosslinking could be beneficial. For example, signaling studies between fibroblasts and keratinocytes or vascularization studies could benefit from the cell-compatible gel. However, for studies that require precise, robust features, EDC crosslinking outperforms the models without crosslinking.

The 3D printed molds can be modified to give a peg-like geometry instead of the ridge-like geometry. The ridge-like geometry was helpful for evaluating the interdigitation index consistently across different stamped features sizes and quantifying the differences. If the models had used a peg geometry, it would have been more difficult to obtain reproducible cross-sections. However, for future applications, it may be beneficial to use a peg-like geometry. In addition to a peg-like geometry, other factors including curvature of pegs could be explored. An advantage of the 3D printing approach is that individual pegs can be fabricated at different shapes and sizes easily, allowing the creation of a variety of different sized posts randomly distributed across the stamp.

It is demonstrated herein that the instant human skin model is compatible with keratinocytes from both a neonatal donor and an adult diabetic donor. However, to create a full diabetic skin disease model, it may be beneficial to include other cell types from diabetic donors, including fibroblasts and endothelial cells²². The present stamping approach can be applied to develop an in vitro skin disease model.

Several recent results in the literature have shown that markers for epidermal stem cells may bias to either the bases or tips of the rete pegs^{23,24}. In the present models, proliferation or stem cell markers were not observed to be expressed differently on the bases or tips of the rete pegs. One reason for this difference could be that here, ridge structures were used rather than peg structures. It is possible that cells need to sense the curvature in multiple directions to organize to the bottoms or tops of pegs. Second, Lawlor et al. (2015) describes many factors that contribute to the maintenance of rete pegs in real human skin, including capillaries, endothelial cells and pericytes, nerves, fibroblasts, and adipocytes⁶. Interactions between keratinocytes and these features could be important in guiding epidermal stem cell migration to different locations in rete pegs. The culture conditions were also different between this work and other work showing epidermal stem cell organization, in which keratinocytes were cultured submerged in media for between 1-4 days on collagen-coated PDMS substrates^{23,24}, whereas here the keratinocytes were cultured on gels for 14 days, and 10 days at the air-liquid interface on a telocollagen-fibrin scaffold. The culture conditions and substrate stiffness could influence epidermal stem cell migration and organization.

Interestingly, spherical growths of proliferative cells was observed in the middle of some of the wells in the EDC-crosslinked samples (see, for example, FIG. 2, day 14+crosslinking, 200 μm and 500 μm; FIGS. 20E and 20G). Without wishing to be bound by theory, it is contemplated herein that these structures are caused by the growth of cells on the walls of the pegs inward.

In the embodiments used in this example, the number of samples generated was generally limited by the number of keratinocytes grown in flasks before preparing the gels. Keratinocytes in this work were used in either passage 3 or passage 4, so increasing the number of keratinocytes would require using more donor cells. In order to generate more distinct samples with the same number of cells, smaller wells could be used. For example, while in this study transwell inserts in 12 well plates with culture area of 1.12 cm² per insert were used, transwell inserts could be used in 24 well plates with culture area of 0.33 cm² per insert. Using the same keratinocyte seeding density, this would generate more than 3× as many samples with the same number of cells. In embodiments where large numbers of samples are needed for a particular application (for example, irritation tests with many test compounds), this can provide a viable approach.

It can be advantageous to minimize the amount of bubbles present in the stamped gels, e.g., by autoclaving the silicone stamps submerged in water, sonicating the beaker containing the stamps to remove air bubbles, and/or coating the stamps in a 2% pluronic F-127 solution before stamping. Alternatively, sampled with bubbles can be identified visually or optically and discarded, e.g., before adding keratinocytes.

The stamping procedure described herein generates rete-peg like structures in human skin models. These structures are robust to degradation over 14 days of culture, with lasting features in the crosslinked and not crosslinked skin models. These skin models express expected patterns of cytokeratins, basement membrane proteins, and proliferation markers. These models can provide in vitro disease models with rete peg structures and irritation testing of human skin models with rete peg structures.

### Methods

Cell culture. Primary human neonatal dermal fibroblasts (ATCC), primary human neonatal keratinocytes (ATCC), and primary human adult diabetic keratinocytes (Lonza) were used in this study. Fibroblasts were cultured in low-serum fibroblast medium (ATCC) and used in experiments from passage number 4-7. Neonatal keratinocytes and diabetic keratinocytes were cultured in CnT-Prime medium (CellnTec) and used in experiments from passage number 3-4. All cells were passaged at approximately 80% confluency by washing with PBS without calcium and magnesium and adding 0.05% trypsin-EDTA for 2-4 minutes. The trypsin was neutralized using 5% FBS in PBS −/−. Cells were centrifuged at 150 rcf for 5 minutes. After passaging, cells were used in experiments or used in continued culture by splitting 1:5 for fibroblasts or 1:3 for keratinocytes.

Silicone stamp fabrication. Molds for silicone stamps were 3D-printed using an Envisiontec Aureus™. 3D-printed molds were washed in isopropyl alcohol once for 15 minutes, and then a second time overnight. After washing the molds were dried and heated in an oven at 80° C. for 15 minutes and cured by exposing to UV light for 5 minutes (Omnicure™). Molds were then plasma treated (Diener Zepto-BL-W6™) for 5 minutes and silanized in a vacuum chamber overnight using trichloro(1H,1H,2H,2H-perfluorooctyl)silane. After silanization, the molds were ready for use. Ecoflex™ 00-50 was prepared by mixing parts A and B in equal proportion in a speed mixer at 2000 RPM for 1 minute. This was poured into the 3D-printed molds and degassed in a vacuum chamber for 10 minutes. The Ecoflex™ was then removed from the vacuum chamber and allowed to cure at room temperature for 1 hour, and then at 80° C. for at least 3 hours.

Telocollagen and fibrin skin model culture. Collagen solutions were prepared by first chilling centrifuge tubes on ice. Second, 10×PBS with phenol red indicator was added to the tubes at 1/10 the final volume of solution. Third, 1M NaOH was added to the tubes to neutralize the collagen. Fourth, telocollagen or atelocollagen was added to the tubes after rinsing serological pipettes and pipette tips with ice cold PBS. The collagen was mixed by pipette until the solution color became uniform at a pH of about 7-7.5. If bubbles were introduced during the mixing step, the collagen tubes were centrifuged at 4° C. and 300 rcf for 1 minute to remove the bubbles. Collagen tubes were kept on ice until needed for the experiment.

Ecoflex™ stamps were sterilized by autoclave submerged in DI water. If bubbles were present near the features of the stamps, the autoclaved beaker was briefly sonicated in a water bath until the bubbles were removed (about 10 seconds). In the biosafety cabinet, the stamps were removed from the beaker and placed face down in a 24 well plate filled with 2% wt/vol pluronic F127 to coat the silicone in a thin layer of pluronic F127. This was found to reduce the likelihood of bubble formation and prevent the gels from sticking to the stamps. The stamps soaked in the 2% pluronic for at least 5 minutes, and then were moved to a 24 well plate filled with PBS to soak until the experiment. Keeping the stamps hydrated by soaking in PBS was necessary to reduce the risk of bubbles in stamped models.

Fibrinogen was prepared in advance by placing in a thin layer in a 60 mm dish and dissolving in PBS without magnesium and calcium at 37° C. for 2 hours at a concentration of 80 mg/mL. The fibrinogen was aliquoted and stored at −20° C. Prior to experiment, the fibrinogen was thawed and warmed to 37° C.

Fibroblasts were passaged and counted using Cell Countess™ (ThermoFisher). Fibroblasts were split into separate centrifuge tubes so that each sample would have approximately 1-2 million cells per mL. Cells were centrifuged at 150 rcf for 5 minutes, and resuspended briefly in the 80 mg/mL fibrinogen. Thrombin was added to the collagen solution to a final concentration of 0.5 U/mL, and then the cells in fibrinogen were added to the collagen solution. The final concentrations all of the components was: 7.9 mg/mL telocollagen or atelocollagen, 4.6 mg/mL fibrinogen, 0.5 U/mL thrombin, 1-2 million cells/mL. The pipette tips were rinsed in ice cold PBS before mixing the cells in the collagen solution. 295 uL of this solution was added to a 12 well plate, and then the Ecoflex stamps were placed on top of the solution. When the 12 well plate was filled with stamped samples, the plate was placed in a 37° C. incubator for 90 minutes to allow the collagen and fibrinogen to gel. After 90 minutes, the stamps were removed and CnT-FTAL medium was added on the samples.

When the keratinocytes were confluent (either the same day or the next day after preparing the stamped gels), keratinocytes were passaged and added on top of the gels at 400,000 cells per insert.

Samples were cultured in CnT-FTAL medium for the duration of culture, with media changes every 2-3 days. During the first four days of culture, 2 mL of medium was added below the insert, and 0.5 mL of medium was added above the gel. On the fourth day following the addition of keratinocytes, samples were moved to the air-liquid interface by placing a 3 mm silicon rubber spacer below the transwell inserts. 2.5 mL of medium was added below the inserts, and any excess medium above the gel was aspirated when samples were treated at the air liquid interface.

Samples were fixed by removing medium and adding 4% formaldehyde in PBS for 1 hour. Samples were then washed in PBS three times, 10 minutes each wash, and then maintained PBS with 0.05% sodium azide for storage before staining.

EDC crosslinked skin model culture. For the EDC-crosslinked skin models, collagen solutions and Ecoflex™ stamp preparation steps were the same as above, with the exception that no cells were used during the stamping process. Similarly, thrombin and fibrinogen were added to the collagen solution, and this solution was pipetted into transwell inserts, and stamps were applied on top of the collagen solutions. These were allowed to gel in an incubator at 37° C. for 90 minutes. After removing the stamps, 2.5 mL of 14 mg/mL EDC in 100% ethanol was added to each of the stamped gels in transwell inserts to crosslink them. Gels were crosslinked at room temperature for 24 hours, and then samples were washed twice in 100% ethanol for ten minutes, and once in 70% ethanol for 24 hours. Samples were next washed in PBS twice for ten minutes and once for 24 hours. At this point, the samples were washed once more in PBS and then in CnT-FTAL medium and placed in the incubator to equilibrate before the addition of cells. Fibroblasts were passaged and seeded at 500,000 cells per insert. Fibroblasts were cultured for 4 days before adding keratinocytes at 400,000 cells per insert. These samples were cultured as described above.

Irritation Test. After keratinocytes had grown for 14 days, 50 μL of SDS at a concentration between 1-3 mg/mL in PBS was applied to the top of each skin sample. 1×PBS was applied as a negative control. Samples were incubated with SDS or PBS for 18 hours in a 37° C. incubator. Samples in transwell inserts were then washed with PBS and moved to a separate 12 well plate with 1 mL of 0.5 mg/mL MTT (3-(4,5-Dimethylthiazol-2-yl)-2,5-Diphenyltetrazolium Bromide) in CnT-FTAL media in each well. The MTT viability assay was run for 3 hours. Then, gel samples were removed from the transwell inserts and transferred to a new 12 well plate with 2.5 mL of isopropanol for at least 18 hours to dissolve the purple formazan substrate. After the substrate was completely dissolved, 50 μL of isopropanol formazan solution was transferred from each well to a 48 well plate, which was measured on a plate reader using the absorbance setting at 490 nm.

Confocal Microscopy. Cross-sections of samples were obtained by removing the samples from the transwell inserts using scalpel and tweezers and slicing samples perpendicular to the stamp's ridged structures. Samples were cut into small rectangular pieces about 2 mm×4 mm. These pieces were placed in a 48 well plate for staining. Samples in FIGS. 18, 20, 22, 25, 27, and 28 were stained with wheat germ agglutinin at 5 μg/mL and DAPI at 0.2 μg/mL overnight and washed in PBS with 0.05% sodium azide before imaging. Other samples in FIGS. 3 and 5 were first permeabilized in 0.5% Triton X-100 in PBS for 30 minutes; second blocked in 5% donkey serum, 0.1% Triton X-100, and PBS overnight; third incubated with primaries (listed below) for 2-3 days in 5% donkey serum, 0.1% Triton X-100 and PBS; fourth washed three times in PBS for 1 hour each wash; and fifth incubated with secondaries (listed below), Actin Red 555 ReadyProbes™ (Thermofisher), and DAPI for 1-2 days in 5% donkey serum, 0.1% Triton X-100 and PBS. Sixth, samples were washed three times in PBS for 1 hour each wash before imaging on a confocal microscope (Zeiss). All primary and secondary antibodies were purchased from Abcam. All primary antibodies were diluted 1:200, and all secondaries were diluted 1:500. Primary antibodies included cytokeratin 10 (rabbit, ab76318), cytokeratin 14 (mouse, ab7800), Ki67 (rabbit, ab15580), p63 (rabbit, ab124762), collagen IV (mouse, ab6311), laminin (rabbit ab11575). Secondary antibodies included donkey anti-rabbit IgG H&L Alexa647 (ab150075) and donkey anti-mouse IgG H&L Alexa488 (ab150105).

Image analysis. Images were analyzed using ImageJ™. Interdigitation index was measured using the segmented line tool and tracing the boundary between the dermis and epidermis layers of the skin models from one end of the image to the other and comparing to a straight line with the same start and end points.

### REFERENCES

1. Bush, K. A. & Pins, G. D. Development of microfabricated dermal epidermal regenerative matrices to evaluate the role of cellular microenvironments on epidermal morphogenesis. Tissue Eng.-Part A 18, 2343-2353 (2012).
2. Clement, A. L., Moutinho, T. J. & Pins, G. D. Micropatterned dermal-epidermal regeneration matrices create functional niches that enhance epidermal morphogenesis. Acta Biomater. 9, 9474-9484 (2013).
3. Blackstone, B. N. et al. Fractional CO2 laser micropatterning of cell-seeded electrospun collagen scaffolds enables rete ridge formation in 3D engineered skin. Acta Biomater. 102, 287-297 (2020).
4. Langton, A. K. et al. Organization of the dermal matrix impacts the biomechanical properties of skin. Br. J. Dermatol. 177, 818-827 (2017).
5. Woodley, D. T. Burn Wounds Resurfaced by Cultured Epidermal Autografts Show Abnormal Reconstitution of Anchoring Fibrils. JAMA 259.
6. Lawlor, K. & Kaur, P. Dermal Contributions to Human Interfollicular Epidermal Architecture and Self-Renewal. Int. J Mol. Sci. 16, 28098-28107 (2015).
7. Jones, P. H., Harper, S. & Watt, F. M. Stem cell patterning and fate in human epidermis. Cell 80, 83-93 (1995).
8. Langton, A. K., Halai, P., Griffiths, C. E. M., Sherratt, M. J. & Watson, R. E. B. The impact of intrinsic ageing on the protein composition of the dermal-epidermal junction. Mech. Ageing Dev. 156, 14-16 (2016).
9. Langton, A. K., Graham, H. K., Griffiths, C. E. M. & Watson, R. E. B. Ageing significantly impacts the biomechanical function and structural composition of skin. Exp. Dermatol. 28, 981-984 (2019).
10. Murphy, M., Kerr, P. & Grant-Kels, J. M. The histopathologic spectrum of psoriasis. Clin. Dermatol. 25, 524-528 (2007).
11. Cubo, N., Garcia, M., del Cañizo, J. F., Velasco, D. & Jorcano, J. L. 3D bioprinting of functional human skin: production and in vivo analysis. Biofabrication 9, 015006 (2016).
12. Derr, K. et al. Fully Three-Dimensional Bioprinted Skin Equivalent Constructs with Validated Morphology and Barrier Function. Tissue Eng. Part C Methods 25, 334-343 (2019).
13. Kim, B. S., Gao, G., Kim, J. Y. & Cho, D. 3D Cell Printing of Perfusable Vascularized Human Skin Equivalent Composed of Epidermis, Dermis, and Hypodermis for Better Structural Recapitulation of Native Skin. Adv. Healthc. Mater. 8, 1801019 (2019).
14. Baltazar, T. et al. Three Dimensional Bioprinting of a Vascularized and Perfusable Skin Graft Using Human Keratinocytes, Fibroblasts, Pericytes, and Endothelial Cells. Tissue Eng. Part A 26, 227-238 (2020).
15. Kim, B. S. et al. Engineering of diseased human skin equivalent using 3D cell printing for representing pathophysiological hallmarks of type 2 diabetes in vitro. Biomaterials 272, 120776 (2021).
16. Suzuki, A. et al. Manufacturing micropatterned collagen scaffolds with chemical-crosslinking for development of biomimetic tissue-engineered oral mucosa. Sci. Rep. 2020 101 10, 1-14 (2020).
17. Malara, M. M. et al. Cultured Epithelial Autograft Combined with Micropatterned Dermal Template Forms Rete Ridges In Vivo. Tissue Eng. Part A ten.tea.2020.0090 (2020) doi: 10.1089/ten.tea.2020.0090.
18. Shen, Z. et al. Construction of tissue-engineered skin with rete ridges using co-network hydrogels of gelatin methacrylated and poly(ethylene glycol) diacrylate. Mater. Sci. Eng. C 129, 112360 (2021).
19. Timár, F., Soós, G., Szende, B. & Horváth, A. Interdigitation index—a parameter for differentiating between young and older skin specimens. Skin Res. Technol. 6, 17-20 (2000).
20. Soares, E. & Zhou, H. Master regulatory role of p63 in epidermal development and disease. Cell. Mol. Life Sci. 75, 1179-1190 (2018).
21. OECD. Test Guideline No. 439: In Vitro Skin Irritation: Reconstructed Human Epidermis Test Methods. 28 (2021).
22. Ozdogan, C. Y. et al. An in vitro 3D diabetic human skin model from diabetic primary cells. Biomed. Mater. 16, 015027 (2021).
23. Mobasseri, S. A. et al. Patterning of human epidermal stem cells on undulating elastomer substrates reflects differences in cell stiffness. Acta Biomater. 87, 256-264 (2019).
24. Viswanathan, P. et al. Mimicking the topography of the epidermal-dermal interface with elastomer substrates. Integr. Biol. U. K. 8, 21-29 (2016).

1. A method comprising:
a) Applying at least one face of a stamp onto at least one surface of a pre-gel comprising at least a first type of cell, wherein the at least one face of the stamp comprises one or more microfeatures;
b) maintaining contact of the at least one face of the stamp with the at least one surface of the pre-gel while the pre-gel forms a gel;
c) separating the at least one face of the stamp and the at least one surface of the gel formed in step b) and thereby providing a gel comprising at least one stamped surface; and
d) contacting the at least one stamped surface of the gel resulting from step c) with at least a second type of cell to provide a two layer 3-D culture.

2.-41. (canceled)

42. A method comprising:
a. preparing a two-layer 3-D culture according to any of the preceding claims;
b. applying a stimulus comprising a candidate agent, mechanical stress, or trauma to the two-layer 3-D culture;
c. and optionally, measuring or observing one or more responses of the two-layer 3-D culture to the stimulus.

43. (canceled)

44. A two layer 3-D culture comprising:
a) A first layer comprising at least a first type of cell in a gel;
b) A second layer comprising at least a second type of cell; and
c) A boundary between the first and second layers comprising one or more microfeatures.

45. The culture of claim 44, wherein:
i) the at least a first type of cell is fibroblasts and the at least a second type of cell is keratinocytes;
ii) the at least a first type of cell is fibroblasts and the at least a second type of cell is keratinocytes and melanocytes;
iii) the at least a first type of cell is fibroblasts and the at least a second type of cell is melanocytes;
iv) the at least a first type of cell is fibroblasts and endothelial cells and the at least a second type of cell is keratinocytes;
v) the at least a first type of cell is fibroblasts and endothelial cells and the at least a second type of cell is keratinocytes and melanocytes;
vi) the at least a first type of cell is fibroblasts and endothelial cells and the at least a second type of cell is melanocytes;
vii) the at least a first type of cell is fibroblasts and immune cells and the at least a second type of cell is keratinocytes;
viii) the at least a first type of cell is fibroblasts and immune cells and the at least a second type of cell is keratinocytes and melanocytes;
ix) the at least a first type of cell is fibroblasts and the at least a second type of cell is induced pluripotent stem cells;
x) the at least a first type of cell is induced pluripotent stem cells and the at least a second type of cell is induced pluripotent stem cells;
xi) the at least a first type of cell is fibroblasts and the at least a second type of cell is cells differentiated from induced pluripotent stem cells;
xii) the at least a first type of cell is cells differentiated from induced pluripotent stem cells and the at least a second type of cell is cells differentiated from induced pluripotent stem cells;
xiii) the at least a first type of cell is fibroblasts and the at least a second type of cell is heptatocytes;
xiv) the at least a first type of cell is fibroblasts and the at least a second type of cell is intestinal stem cells;
xv) the at least a first type of cell is fibroblasts and the at least a second type of cell is intestinal epithelial cells;
xvi) the at least a first type of cell is fibroblasts and the at least a second type of cell is tumor cells
xvii) the at least a first type of cell is fibroblasts from a psoriasis patient and the at least a second type of cell is keratinocytes from a psoriasis patient; or
xviii) the at least a first type of cell is fibroblasts from a diabetic patient and the at least a second type of cell is keratinocytes from a diabetic patient.

46. The culture of claim 44, wherein the at least a first type of cell comprises fibroblasts and the at least a second type of cell comprises keratinocytes.

47. The culture of claim 46, wherein the keratinocytes form a stratified epidermis layer or a basement membrane is present.

48. (canceled)

49. (canceled)

50. (canceled)

51. The culture of claim 44, wherein the second layer is at least 50 μm in depth.

52. The culture of claim 44, wherein the first layer comprises one or more of:
collagen; atellocollagen; telocollagen; collagen methacrylate; polyethylene glycol (PEG); PEG-DA; fibrin; fibrinogen; gelatin; agarose; thrombin; PBS; NaOH; Phenolphthalein; or a combination of any of the foregoing.

53. (canceled)

54. The culture of claim 52, wherein the first layer comprises telocollagen and fibrinogen.

55. The culture of claim 52, wherein the first layer does not comprise atelocollagen.

56. The culture of claim 52, wherein the first layer comprises more than 6 mg/mL of collagen, atellocollagen, or telocollagen.

57. The culture of claim 52, wherein the first layer comprises more than 8 mg/mL of collagen, atellocollagen, or telocollagen.

58. The culture of claim 52, wherein the first layer has a depth of from about 100 μm to 10 mm.

59. (canceled)

60. (canceled)

61. (canceled)

62. The culture of claim 44, wherein each microfeature extends 1 μm to 1 mm into one of the layers.

63. (canceled)

64. The culture of claim 4, wherein each microfeature has a width and/or length of at least 100 μm.

65. The culture of claim 44, wherein each microfeature has a height which is no more than 5× the narrowest dimension of the microfeature.

66. The culture of claim 44, wherein each microfeature forms a shape that is a polyhedron, a hexahedron, a cube, a ridge, a wall, a shape created by introducing a curve or angle into any of the preceding shapes, or a shape created by rounding one or more of the edges of one of the preceding shapes.

67. The culture of claim 44, wherein the culture does not comprise HaCaT keratinocytes or PEG-diacrylate.

68.-73. (canceled)

74. A two layer 3-D culture comprising:
a. a first layer comprising at least a first type of cell in a gel comprising telocollagen, fibrinogen, NaOH, and thrombin; and
b. a second layer comprising at least a second type of cell.

75.-103. (canceled)

104. A kit comprising one or more stamps comprising at least one face comprising one or more microfeatures.

105. (canceled)

106. (canceled)
